(19) United States
(12) Patent Application Publication
(10) Pub. No.: US 2008/0039410 A1
(43) Pub. Date: Feb. 14, 2008

(54) Inositol 1,4,5-Trisphosphate Receptor Mutants and Uses Thereof

(76) Inventors: David I. Yule, Penfield, NY (US); Larry Wagner II, Rochester, NY (US)

Correspondence Address: NEEDLE & ROSENBERG, P.C., SUITE 1000, 999 PEACHTREE STREET, ATLANTA, GA, 30309-3915, US

(21) Appl. No.: 10/587,300

(22) PCT Filed: Jan. 26, 2005
(86) PCT No.: PCT/US05/02380

§ 371 Date: May 8, 2007

Related U.S. Application Data
(60) Provisional application No. 60/539,245, filed on Jan. 26, 2004.

(51) Int. Cl. A61K 31/7052; A61P 19/02; A61P 31/18; A61P 43/00; C07H 21/00; C07K 14/705; C12N 15/63; C12N 5/10; C12Q 1/02

(52) U.S. Cl. 514/44; 435/29; 435/320.1; 435/325; 530/395; 536/23.5

(57) ABSTRACT

Disclosed herein are compositions and methods for treating subjects with conditions related to Inositol 1,4,5-trisphosphate receptor (InsP3R). Described herein are mutants of InsP3R, methods of making mutants, and methods of using InsP3R mutants for the treatment of a disease or condition.

This invention was made with government support under Grants RO1-K54568; RO1-DE14756 and PO1-DE13539 awarded by the National Institutes of Health. The government has certain rights in the invention.

### I. BACKGROUND OF THE INVENTION

Inositol 1,4,5-trisphosphate receptors are intracellular ion channels that function to couple the activation of cell surface receptors for neurotransmitters, hormones, and growth factors to the initiation of intracellular Ca²⁺ release (1). Three genes have been cloned that encode distinct proteins of a molecular mass of ˜300 kDa, named the type 1 (InsP₃R-1),1 type 2 (InsP₃R-2), and type 3 (InsP₃R-3) InsP3Rs (2-5). In addition, multiple receptor proteins with distinct tissue distributions are produced by alternate splicing of the type 1 receptor gene (7, 90). Notably, the type-1 receptor gene is alternatively spliced to yield additional variants of the receptor, which have specific tissue distribution [6, 7]. Most cells express multiple isoforms of InsP₃R (91). Furthermore, the expression level and complement of receptors differ in individual tissues, and this together with regulation of the activity of the channel is thought to be a major determinant of the rich diversity of Ca²⁺ signaling events observed in cells (91, 12).

The functional channel is formed co-translationally by the tetrameric association of four individual receptor subunits (92, 93). Each subunit has a binding site for InsP3 toward the N terminus formed by a cluster of positively charged amino acids thought to coordinate the negatively charged phosphate groups of InsP₃ (8, 9, 94). The C terminus of each subunit is postulated to span intracellular membranes six times and forms a single cation-selective pore (10, 11, 95). In addition, this region signals retention of the protein to the endoplasmic reticulum (96, 97).

Although the InsP₃-binding pocket and channel pore are highly conserved between InsP₃R family members, the intervening sequence between the binding region and pore is more divergent and consists of the so-called “regulatory and coupling” or “modulatory” domain. This region, consisting of 1600 amino acids, is thought to be important in modulating the Ca²⁺ release properties of the InsP₃R. Indeed, Ca²⁺ release through the InsP₃R is markedly influenced by many factors, most importantly by Ca²⁺ itself (98). InsP₃R activity is also influenced through interaction with numerous factors such as proteins, adenine nucleotides, and phosphorylation and in particular by cyclic nucleotide-dependent kinases (12).

This regulation of InsP₃R, together with the complement of InsP₃R types and the sub-cellular localization of the channel, are thought to be the major determinants of the spatio-temporal characteristics of agonist-evoked Ca²⁺ signals [13, 14]. These particular characteristics likely contribute to the fidelity and specificity associated with activation of Ca²⁺ -dependent effectors. The most important regulator of InsP₃-induced Ca²⁺ release is Ca²⁺ itself [12, 15-17], however, numerous factors including adenine nucleotides [18, 19], protein interactions [12, 20] and phosphorylation by various kinases can significantly influence InsP₃R function [7, 21-31]. In particular, since all InsP₃R subtypes are phosphorylated by cAMP and cGMP-dependent protein kinases (PKA and PKG) [7, 21-23, 25, 26, 28, 29, 32-41], the InsP₃R may represent an important nexus for cross-talk between these distinct signaling pathways. Indeed, cyclic nucleotide-dependent kinase-induced phosphorylation of InsP₃R is proposed to be important in such diverse physiological and pathophysiological processes as synaptic plasticity [23], remodeling following neurotoxic insult [39], smooth muscle contractility [36, 37] and fluid secretion [29].

### II. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows the phosphorylation of S1755results in enhanced Ca²⁺ release by S2⁺ InsP₃R-1. In FIG. 1A, DT40 3ko cells shown in the inset were transfected with M3 receptor, HcRed and S2⁺ InsP₃R-1 as described below. Fura-2 loaded cells were stimulated with 50 nM Carbadol (CCh) to increase [Ca²⁺]ᵢ. A [Ca²⁺]ᵢ increase was only evoked in cells expressing HcRed, and thus presumably M3 receptor and S2⁺ InsP₃R-1 (compare black trace vs. gray trace from cells indicated in the inset). Treatment with 20 μM forskolin, to raise cAMP levels and activate PKA, resulted in a markedly enhanced CCh-induced [Ca²⁺]ᵢ signal. After removal of forskolin a subsequent exposure to CCh resulted in a [Ca²⁺]ᵢ increase similar to control. In FIG. 1B, a similar experimental paradigm was utilized in DT-40 3ko cells expressing S1589A S2⁺ InsP₃R-1. Stimulation with CCh following 20 μM forskolin also resulted in a markedly enhanced [Ca²⁺]ᵢ increase relative to a control stimulation. FIG. 1C shows that in cells expressing S1755A S2⁺ InsP₃R-1, forskolin treatment did not result in an enhanced signal. FIG. 1D shows pooled data from the number of cells indicated for each construct, showing the normalized fold increase in initial [Ca²⁺]ᵢ peak over control resulting from forskolin treatment of cells expressing S2⁺ InsP₃R-1 and serine to alanine mutants. Forskolin treatment resulted in CCh responses in Wild type and S1589A S2^{+ InsP}₃R-1 cells being significantly different from S1755A S2^{+ InsP}₃R-1 expressing cells. Cartoon inset depicts the S2^{+ InsP}₃R-1 regulatory and coupling domain. The black shaded region represents the S2 splice region. The functionally important phosphorylation of S1755is indicated by a gray circle.

FIG. 2 shows the potentiation of Ca²⁺ release by forskolin after flash photolysis of ciInsP₃.PM. DT-40 3ko cells expressing S2^{+ InsP}₃R-1 were loaded with the visible wavelength Ca²⁺ indicator Fluo-4 and the cell permeable caged InSP₃ analog ciInsP₃-PM as described below. FIG. 2A shows minimal photolysis of InsP₃ was evoked by a brief UV flash, (˜0.5 msec indicated by the arrows) resulting in a small increase in [Ca²⁺]ᵢ. A subsequent, identical flash of UV light 5 min. later fails to evoke a larger increase in [Ca²⁺]ᵢ, although increasing the duration of the flash to 5 ms evokes a significantly larger increase in [Ca²⁺]ᵢ (arrow, “max uncage”). In FIG. 2B, an identical protocol was followed except the second minimal uncaging was performed following 5 min. treatment with 10 μM forskolin. This treatment resulted in a significantly larger increase in [Ca²⁺]ᵢ when compared to the initial uncaging. FIG. 2C shows pooled data comparing normalized fold increase for the first and second uncaging in the presence or absence of forskolin. Treatment with forskolin results in a statistically significant increase in the second response.

FIG. 3 shows both S1589and S1755 are functionally important PKA phosphorylation sites in S2⁻ InsP₃R-1. A similar experimental paradigm as described in FIG. 1 was utilized to assess the consequences and functionally important phosphorylation sites in S2⁻ InsP₃R-1. FIG. 3A shows treatment of wild type S2⁻ InsP₃R-1 with forskolin resulted in enhanced CCh stimulated Ca²⁺ release with respect to a control CCh stimulation. FIG. 3B shows a similar potentiation was observed with S1589A S2⁻ InsP₃R-1 expressing cells. FIG. 3C shows a similar enhanced [Ca²⁺]ᵢ signal was observed following forskolin treatment in S1755A S2⁻ InsP₃R-1 expressing cells. FIG. 3D shows that no effect of forskolin treatment was observed in double mutant S1589A/S1755A S2⁻ InsP₃R-1 expressing cells. FIG. 3E shows pooled data for the number of cells indicated for each construct. The filled bars indicate data for the particular construct obtained using the low affinity Ca²⁺ indicator Fura-2FF. Normalized fold increase is only significantly altered in the double mutant. Cartoon inset depicts the functionally important phosphorylation of S1589 and S1755, indicated by gray circles.

FIG. 4 shows the phosphorylation of S1755 by PKG results in enhanced Ca²⁺ release by S2⁺ InsP₃R-1. A similar experimental paradigm utilized for experiments presented in FIG. 1 was performed to assess the effects and site(s) of phosphorylation by PKG on S2⁺ InsP₃R-1. FIG. 4A shows treatment with 10 μM 8-Br cGMP to specifically activate PKG results in a marked potentiation of CCh-evoked Ca²⁺ release when compared to control CCh stimulation in the absence of PKG activation. FIG. 4B shows a similar potentiation of Ca²⁺ release following PKG activation was observed in cells expressing S1589A S2⁺ InsP₃R-1. FIG. 4C shows that PKG activation does not enhance Ca²⁺ release by S1755A S2⁺ InsP₃R-1. FIG. 4D shows that PKG does not inhibit CCh-induced Ca²⁺ release by S1755A S2⁺ InsP₃R-1. FIG. 4E shows pooled data for the number of cells indicated for each construct. Normalized fold increase by S1755A S2⁺ InsP₃R-1 was significantly different from both wild-type and S1589A S2⁺ InsP₃R-1. Cartoon inset depicts the functionally important phosphorylation of S1755, indicated by a gray circle.

FIG. 5 shows that treatment with PKI inhibits forskolin but not 8-Br cGMP-induced potentiation. Cells expressing S2⁺ InsP₃R-1 were treated for 30 mins with myr-PKI(14-22) prior to assessing the effects of activating PKA or PKG. FIG. 5A shows that PKI treatment completely abolishes the forskolin-induced enhancement of Ca²⁺ release. FIG. 5B shows that PKI does not affect the 8-Br cGMP-induced enhancement of Ca²⁺ release. FIG. 5C shows the results of pooled data.

FIG. 6 shows PKG activation is without effect on Ca²⁺ release by S2⁻ InsP₃R-1. A similar experimental paradigm used in experiments depicted in FIG. 1 was utilized to assess the effects of PKG phosphorylation of S2⁻ InsP₃R-1. FIG. 6A shows that PKG activation has no effect on CCh-evoked Ca²⁺ release by wild-type S2⁻ InsP₃R-1. FIG. 6B shows that similarly no effect was observed in S1589A S2⁻ InsP₃R-1 expressing cells. FIG. 6C shows that no effect was observed in S1755A S2⁻ InsP₃R-1 expressing cells. FIG. 6D shows pooled data for the number of cells indicated for each construct. Cartoon depicts absence of phosphorylation by PKG.

FIG. 7 shows in FIG. 7A that ATP treatment of fura-2 loaded mouse parotid acinar cells, in the presence of la³⁺ to block Ca²⁺ entry (isolating P2Y receptors) results in an increase in [Ca²⁺]ᵢ. The [Ca²⁺]ᵢ is markedly enhanced by incubation with forskolin. FIG. 7B shows that CCh treatment of human parotid acinar cells results in an increase in [Ca²⁺]ᵢ which is potentiated by forskolin treatment. Representative traces from >4 experiments and >3 preparations of tissue.

FIG. 8A depicts representative traces showing that mutation of both functional phosphorylation sites in the short form of InsP₃R-1 to glutamate residues results in a receptor which is apparently more sensitive to InSP₃ as revealed by increased sensitivity to CCh. FIG. 8B shows the pooled data illustrating that the S1589E/S1755E mutant InsP₃R-1 is approximately 7.5 fold more sensitive than the wild type S2− InsP₃R-1 and approximately 35 fold more sensitive than the S1589A/S1755A nonphosphorylatable S2−InsP₃R-1

FIG. 9 shows single channel records from an isolated Cos-7 cell nucleus patched on several occasions. K⁺ is the charge carrier, holding potential was +20 mV. Channel activity is observed only when InSP₃ is present in the pipette. Note in the final trace several channels appear to be present. Representative of 7 experiments.

FIG. 10 shows a cartoon depicting the proposed structure of the InsP₃R.

FIG. 11 depicts the regulatory and coupling domain of the InsP₃R-1; showing the phosphorylation sites at S1589 and S1755. In addition the location of the S2 splice site is shown.

FIG. 12A shows that single mutation of S1589E in S2+ InsP₃R has little effect on the potentiation of Ca²⁺ signaling seen upon phosphorylation of the receptor, confirming in the S2+ variant of the receptor that this site is not functional. FIG. 12B/C show that stimulation of cells expressing the S1755E phosphomimetic mutation are apparently more sensitive to stimulation, and in additon no further enhancement of Ca²⁺ signaling is observed following PKA activation, confirming S1755 as the functionally important site.

FIG. 13A/B shows that if either S1589or S1755 is mutated to glutamate individually that no further potentiation by PKA stimulation is observed. Indicating that phosphorylation of individual sites is not functionally additive.

FIG. 14 shows that InsP₃R-III can be phosphorylated in a PKA dependent fashion.

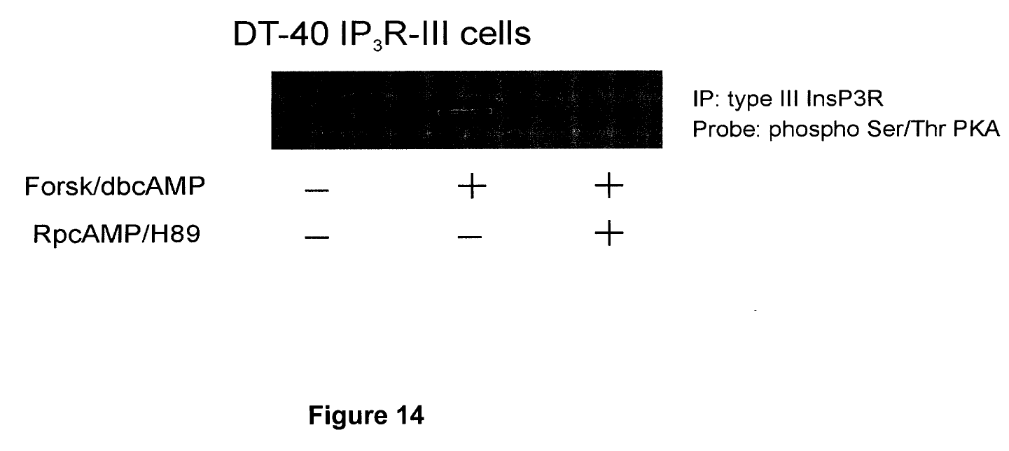

FIG. 15A shows stimulation of DT40 cells expressing chicken InsP₃ R-III results in Ca²⁺ oscillations. FIG. 15B shows that activation of PKA during these oscillations results in an inhibition of the Ca²⁺ signal, consistent with an effect on the InsP₃R-III.

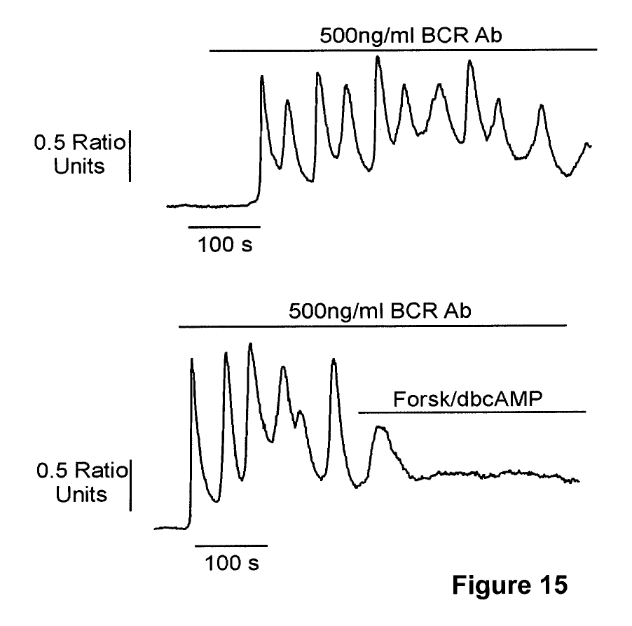

FIG. 16 shows that stimulation of DT-40 3ko transfected with rat InsP₃R-III results in Ca²⁺ signals which are inhibited by stimulating PKA.

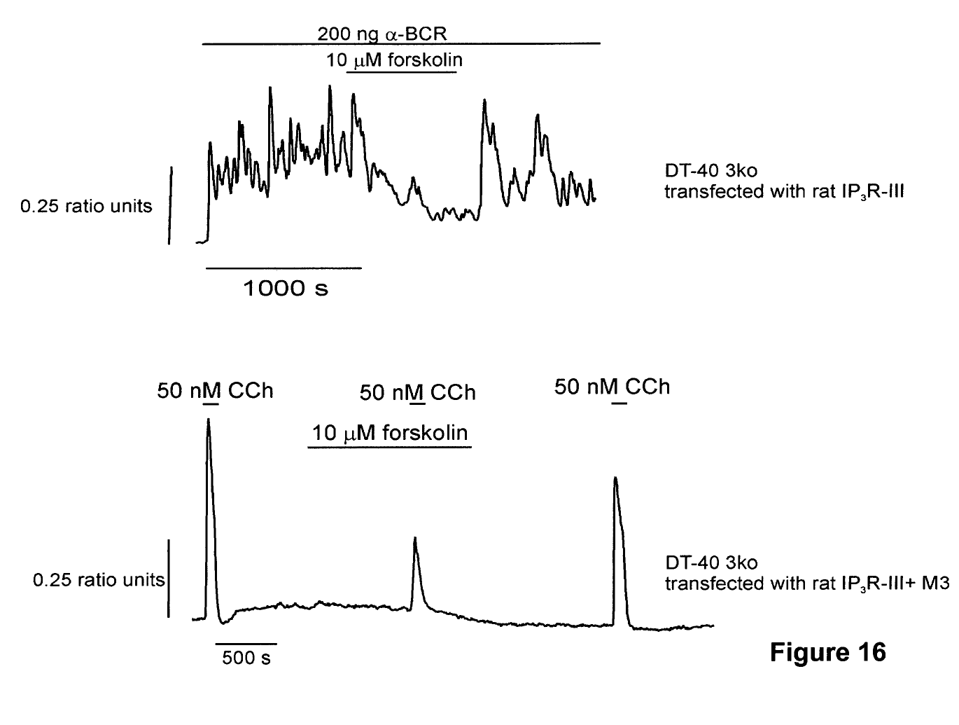

FIG. 17 shows flash photolysis of caged InsP₃ reveals that phosphomimetic InsP₃R-1 exhibits enhanced sensitivity to InsP₃. FIG. 17A represents a schematic of amino acids 1586-1755 of the S2⁺ InsP₃R-1 showing the S2 splice site and potential PKA/PKG phosphorylation sites. Dt-40 3ko cells were transfected with either phosphomimetic mutations of InsP3R-1 (B and D) or nonphosphorylatable mutations (C and E). Experiments in B and C are representative traces from cells transfected with the S2− InsP₃R-1 and in D and E with S2+ InsP₃R-1. The cells were loaded with the visible wavelength Ca²⁺ indicator fluo-4 and the cell-permeable caged InsP₃R analogue ci-IP₃-PM as described under “Experimental Procedures.” Increasing iIP₃ concentrations were generated by varying the duration (energy magnitude) of UV flash exposure from 0.5 to 5 mss. B, cells transfected with S2−EE constructs respond at a lower threshold of UV light exposure and with more robust elevations in [Ca²⁺]ᵢ than cells transfected with S2−AA mutations that are nonphosphorylatable (C). Similarly, as shown in D, S2+SE mutations are more sensitive than S2+SA mutations. The pooled data, expressed as fold increase in the ΔF/F₀ fluorescence, are shown in F (S2⁻ constructs) and G (S2+ constructs). The magnitude of responses to 1.25- and 2.5-ms exposure to UV light was significantly greater in phosphomimetic mutations in each splice variant, p 0.005. The values in parentheses indicate the number of cells responding above a 0.05% ΔF/Fₒ threshold in relation to the number of cells tested. For amino acid sequences shown, RRDS corresponds to SEQ ID NO: 25, RRES to SEQ ID NO: 26, RRDE to SEQ ID NO: 27, RREE to SEQ ID NO: 28, RREA to SEQ ID NO: 29.

FIG. 18 shows the InsP₃ sensitivity of S2⁻InsP₃R-1 and phosphomimetic mutations. Concentration-response relationships for CCh-induced Ca²⁺ signals were examined in Dt-40 3ko cells expressing m3 receptors and InsP₃R-1 constructs. FIG. 18A shows that cells transfected with S2−EE were stimulated with increasing concentrations of CCh as indicated (n=9 cells). A similar paradigm was performed for cells expressing S2−WT (n=10) (B) and for cells expressing S2−AA constructs (n=9) (C). The magnitude of the initial peak (as an indicator of Ca²⁺ release) for each response was normalized to the maximum response in each cell. The pooled data and the fit that describes each concentration-response relationship for each construct is shown in D and illustrates that the sensitivity of Ca²⁺ release was greatest in S2−EE followed by S2−WT and S2−AA. For amino acid sequences shown, RRDE corresponds to SEQ ID NO: 27, RREE to SEQ ID NO: 28, RRDS to SEQ ID NO: 25, RRES to SEQ ID NO: 26, RRDA to SEQ ID NO: 30, and RREA to SEQ ID NO: 29.

FIG. 19 shows the functional sensitivity of single phosphomimetic mutations in S2−InsP₃R-1. FIG. 19A shows that concentration-response relationships were generated exactly as shown in FIG. 18D and as described under “Experimental Procedures.” Cells expressing either S2−SE (n−6) or S2−ES (n=5) displayed intermediate sensitivity between S2−WT and S2−EE constructs. Dotted lines indicate S2−WT and S2−EE for comparison (data from FIG. 18D). FIG. 19B shows that the threshold CCh-stimulated Ca²⁺ release is markedly potentiated by activation of PKA following forskolin treatment in cells expressing S2−WT. In contrast, no potentiation of threshold CCh-stimulated Ca²⁺ release is observed in cells expressing S2−SE in 19D, S2−ES in 19C, or S2−EE in 19E. Pooled data are shown in 19F; potentiation by forskolin is only seen in S2−WT. Numbers in parentheses indicate the number of analyzed cells. For amino acid sequences shown, RRDS corresponds to SEQ ID NO: 25, RRES to SEQ ID NO: 26, RRDE to SEQ ID NO: 27 and RREE to SEQ ID NO: 28.

FIG. 20 shows the phosphorylation of S2−InsP₃R-1 mutants. The phosphorylation status of S2−InsP3R-1 constructs was assessed as described under “Materials and Methods.” FIG. 20A shows that incubation with 20 μM forskolin results in phosphorylation of Ser-1755 in S2−InsP3R-1. No phosphorylation is seen in S2−AA-transfected cells or untransfected HEK-293 cells (UN). FIG. 20B shows that forskolin treatment results in phosphorylation of Ser-1755 in S2−InsP3R-1 but not S2−EE, S2−ES, or S2−SE. The lack of phosphorylation of Ser-1755 in the S2−ES mutant is interpreted as indicating that mimicking phosphorylation of Ser-1589 by phosphomimetic substitution precludes the phosphorylation of Ser-1755 consistent with the functional data shown in FIG. 19. IP, immunoprecipitation; WB, Western blot.

FIG. 21 shows the functional sensitivity of S2^{+ InsP}₃R-1. Concentration-response relationships were generated exactly as described previously. FIG. 21A shows the normalized relationships for S2+WT (n=11), S2+SE (n=6), and S2+SA (n=5) are shown. The fits illustrate the increased sensitivity of the S2+SE relative to S2+WT and S2+SA constructs. FIG. 21B shows the normalized concentration-response relationship for S2+ES is shown with S2+SE and S2+WT (dotted lines for comparison). These data indicate that the sensitivity of S2+ES is similar to S2+WT.

FIG. 22 shows the effects of PKA phosphorylation on S2+ InsP₃R-1 single glutamate substitution constructs. Threshold CCh-stimulated Ca²⁺ release is markedly potentiated by activation of PKA following forskolin treatment in S2+WT-expressing cells (A) or S2+ES (B). FIG. 22C shows, in contrast, no potentiation of threshold CCh-stimulated Ca²⁺ release is observed in cells expressing S2+SE. FIG. 22D shows, pooled data illustrating that S2+ES has properties identical to S2+WT, and thus phosphorylation of this residue is unlikely to impact Ca²⁺ release. Numbers in parentheses indicate the number of analyzed cells.

FIG. 23 shows the effect of PKG activation in single phosphomimetic constructs. Threshold CCh-stimulated Ca²⁺ release is markedly potentiated by activation of PKG following 8-Br-cGMP treatment in S2+WT (A) or S2+ES-expressing cells (B). FIG. 23C shows, in contrast. no potentiation of threshold CCh-stimulated Ca²⁺ release is observed in S2+SE-expressing cells. FIG. 23D shows pooled data illustrating that S2+ES has properties identical to S2+WT, and thus phosphorylation of this residue following activation of PKG is unlikely to impact Ca²⁺ release. Numbers in parentheses indicate the number of analyzed cells.

FIG. 24 shows a comparison of Ca²⁺ oscillations in Dt-40 3ko cells expressing S2−WT or phosphomimetic S2− constructs. Dt-40 3ko cells were transfected with either S2−WT (left panel) or S2−EE construct (middle panel). FIG. 24A shows cells that were stimulated with a threshold concentration (250 ng/ml) of α-IgM antibody to stimulate the B cell receptor. In S2−WT-expressing cells, this resulted in a single Ca²⁺ transient after a long latency (A, left panel). In contrast, multiple Ca²⁺ transients were elicited in S2−EE-expressing cells following a shorter latency. FIG. 24B shows cells that were stimulated with 500 ng/ml α-IgM antibody. Cells transfected with either S2−WT or S2−EE exhibited multiple Ca²⁺ transients of similar frequency, although the initial peak was generally larger and latency shorter in S2−EE-expressing cells. FIG. 24C shows the pooled data from cells stimulated with various concentrations of α-IgM for frequency of oscillations is shown. FIG. 24D is shown for latency, and 8E, is shown for the magnitude of the initial peak. Numbers in parentheses indicate the number of analyzed cells.

FIG. 25 shows Fura-2 loaded DT-403ko cells transfected with M3R, Hc-red to identify transfected cells, and either wild-type (wt) (A) or PKA- phosphorylation site mutant S2⁻ InsP₃R-I (B-D). Forskolin treatment results in potentiation of CCh responses in cells expressing wt, S1589A (SA) or S1755A (AS) mutants (A,B, and C respectively). Mutation of both putative PKA-phosphorylation sites results in loss of the potentiation (AA) (D).

FIG. 26 shows Dt-403ko cells transfected with m3 receptor, InsP₃R-2 and HcRed (red trace) and untransfected cell (black trace). Incubation with forskolin (to activate PKA) but not 8-Br-cGMP (to activate PKG) results in a marked potentiation of carbachol-induced Ca²⁺ signaling.

FIG. 27A shows the sequence of the S2+ InsP₃R-1 and the region that is spliced out in the S2− InsP₃R-1 (shown in red). Splicing of the receptor creates a novel nucleotide binding motif (GXGXXG SEQ ID NO: 24). To disrupt the motif a glycine to alanine mutation was constructed at amino acid 1690. As shown in FIG. 27B in Dt-403ko cells expressing this construct, PKA activation fails to potentiate Ca²⁺ release signal induced by carbachol. For amino acid sequences shown, RRDS corresponds to SEQ ID NO: 25, RRES to SEQ ID NO: 26, GYGEKQISIDELENAELPQPPEAENSTEELEPSPPLRQLEDHKRGEAL to SEQ ID NO: 31, GYGEKGEAL to SEQ ID NO: 32, and GXGXXG to SEQ ID NO: 24.

FIG. 28 shows HEK-293 cells that were transfected with either wild type (WT) or nucleotide binding site mutant (G1690A) and treated with forskolin to activate PKA. The cells were subsequently lysed, proteins separated on SDS page and western blotted using an antibody which recognizes phosphorylated ser 1755 (top panel) or non-phosphorylated ser 1755 (bottom panel). The G1690A mutant is not phosphorylated following forskolin treatment.

### III. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

“Treatment” or “treating” means to administer a composition to a subject with an undesired condition or at risk for the condition. The condition can be any pathogenic disease, autoimmune disease, cancer or inflammatory condition. The effect of the administration of the composition to the subject can have the effect of but is not limited to reducing the symptoms of the condition, a reduction in the severity of the condition, or the complete ablation of the condition.

By “effective amount” is meant a therapeutic amount needed to achieve the desired result or results, e.g., increasing or decreasing Ca²⁺ release, enhancing or blunting physiological functions, altering the qualitative or quantitative nature of the proteins expressed by cell or tissues, and eliminating or reducing disease causing molecules and/or symptoms.

“Optional” or “optionally” means that the subsequently described event or circumstance may or may not occur, and that the description includes instances where said event or circumstance occurs and instances where it does not.

By “subject” is meant an individual. Preferably, the subject is a mammal such as a primate, and, more preferably, a human. The term “subject” can include domesticated animals, such as cats, dogs, etc., livestock (e.g., cattle, horses, pigs, sheep, goats, etc.), and laboratory animals (e.g., mouse, rabbit, rat, guinea pig, etc.).

By “InsP₃R” is meant an inositol 1,4,5-triphosphate receptor. Such receptors are generally the major route of intracellular calcium release in eukaryotic cells and are pivotal for stimulation of calcium dependent effectors. Modulation of calcium release through these receptors has important consequences in development and in a variety of normal and pathological cellular conditions. There are three major types of InsP₃R: InsP₃R-1, InsP₃R-2, and InsP₃R-3. InsP₃R-1 has two major splice variants: the S2⁻ and the S2⁺. The S2⁻ splice variant of InsP₃R-1 is the short splice variant in which 40 amino acids are excised. Specifically, residues 1693 to 1732 of the full length variant (ie. S2⁺ or the long splice variant) are excised. The S2⁻ variant is located predominantly in peripheral tissues whereas the S2⁺ variant is present predominantly in the CNS.

By “homology” is meant the degree of relatedness shared between two or more nucleic acids, peptides, polypeptides or proteins as determined by their sequence structure or function.

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### Mutants

The present invention provides mutant receptor proteins of two general categories: phosphomimetic mutant InSP₃ receptors and nonphosphorylatable mutant InsP₃. By “phosphomimetic” is meant a receptor that has an increased Ca²⁺ release function as compared to the wild-type InsP₃R as a result of amino substitution to mimic phosphorylation. Preferably, the phosphomimetic mutant has a Ca²⁺ release function that is 3, 4, 5, 6, 7, 8, 9, 10 (or any amount in between) times that of the corresponding wild-type receptor. Preferably, the phosphomimetic mutant has a Ca²⁺ release function that is 10, 20, 30, 40 (or any amount in between) times that of the corresponding nonphosphorylatable mutant. By “nonphosphorylatable” mutant is meant a “null” mutant that is not phosphorylated under conditions that cause phosphorylation in the wild-type. The mutants can be derived from InsP₃R-1, either the S2⁻ or the S2⁺ variant; InsP₃R-2; or InsP₃R-3.

By increased or enhanced Ca²⁺ release function is meant an increase release of calcium following a stimulus that activates the InsP₃R. Such stimuli include, for example, carbachol, an analog of acetylcholine, acting at muscarinic M3 receptors or alternatively, any agonist acting at any one of over one hundered plasma membrane receptors for neurotransmitters, hormones and growth factors coupled to the formation of InsP₃. In addition ehanced Ca²⁺ release can occur following direct activation of InsP₃R with InsP₃ or its analogs.

The phosphomimetic mutants are derived by substitution of a serine in a phosphorylation site with a negatively charged amino acid residue. “By substitution of a serine in a phosphorylation site with a different amino acid residue” is meant that serine is removed and the different amino acid residue replaces it. By “a negatively charged amino acid residue” is meant that when incorporated into the protein it provides a net negative charge at the phosphorylation site. Thus, the substitution with the negatively charged amino acid residue neutralizes the positive charge at the site (provided at least by the typical arginine residue at the phosphorylation site). The phosphorylation site can be the strong PKA recognition motif of RXXS (SEQ ID NO:21), in which X represents any amino acid. The serine residue can be replaced with either aspartate or glutamate. Thus the invention provides a InsP₃R mutant comprising at least one substitution of serine with a negatively charged amino acid residue at a phosphorylation site of a wild-type InsP₃R, wherein the mutant has an enhanced Ca²⁺ release function as compared to the wild-type InsP₃R. Preferably the mutant's Ca²⁺ release function is at least 5 times greater than the Ca2+ release function of the wild-type InsP₃R. Preferably the mutant's Ca²⁺ release function is at least 10 times greater than the Ca²⁺ release function of the wild-type InsP₃R.

The invention provides a phosphomimetic InsP₃R-1 mutant, which has enhanced Ca²⁺ release function as compared to the wild-type InsP₃R-1. More specifically, the mutant comprises at least one substitution of serine with a negatively charged amino acid residue at a phosphorylation site, wherein the phosphorylation site is selected from residue 1589 or 1755 of a wild-type InsP₃R sequence. As used throughout, the amino acid residues are numbered according to the rat sequences for the full length InsP₃R. Thus, one of skill in the art, can readily align the sequence for human (ATCC Acc. No. NM_{—}002222), mouse (ATCCAcc. No. NM_{—}010585), or any other species and replace the comparable serine residue with the desired amino acid.

Examples of InsP₃R-1 phosphomimetic mutants include those wherein the substitution of serine with the negatively charged amino acid (including either glutamate or aspartate) is at residue 1589. Thus the mutant can comprise the amino acid sequence of SEQ ID NO:1, which corresponds to the short splice variant S1589E; SEQ ID NO:2, which corresponds to the long splice variant for S1589E; SEQ ID NO:3, which corresponds to S1589D in the short splice variant; SEQ ID NO:4, which corresponds to the long splice variant of S1589D.

The invention also provides the amino acids of SEQ ID NO:1, 2, 3, or 4 having one or more conservative amino acid substitutions, wherein the Ca²⁺ release function is maintained. Also provided are mutants comprising amino acid sequences having at least 80, 85, 90, 95, 96, 97, 98, 99% (or any amount in between these values) homology to the amino acid sequence of SEQ ID NO:1, 2, 3, or 4 wherein the Ca²⁺ release function is maintained. Also provided are the comparable S1589E and S1589D mutants for various species.

Examples of InsP₃R-1 phosphomimetic mutants include those wherein the substitution of serine with the negatively charged amino acid (including either glutamate or aspartate) is at residue 1755. Thus the mutant can comprise the amino acid sequence of SEQ ID NO: 5; which corresponds to the short variant of S1755E; SEQ ID NO:6, which corresponds to the long splice variant of S1755E; SEQ ID NO:7, which corresponds to the short variant of S1755D; and SEQ ID NO:8, which corresponds to the long splice variant of S1755D.

The invention also provides the amino acids of SEQ ID NO: 5, 6, 7, and 8 having one or more conservative amino acid substitutions, wherein the Ca²⁺ release function is maintained. Also provided are mutants comprising amino acid sequences having at least 80, 85, 90, 95, 96, 97, 98, 99% (or any amount in between these values) homology to the amino acid sequence of SEQ ID NO:5,6,7, or 8, wherein the Ca²⁺ release function is maintained. Also provided are the comparable S1755E and S1755D mutants for various species.

The invention also provides an InsP₃R-1 mutant, wherein the substitutions of serine with the negatively charged amino acid is at residues 1589 and 1755. Either glutamate or aspartate is substituted for the two serines, in any combination. Thus, the invention provides a mutant, wherein glutamate is substituted for serine at residues 1589 and 1755; including for example a mutant comprising the amino acid sequence of SEQ ID NO:9, which corresponds to the S1589E/S1755E mutant of the short splice variant, or SEQ ID NO:10, which corresponds to the S1589E/S1755E mutant of the long splice variant. The invention also provides a mutant, wherein aspartate is substituted for serine at residues 1589 and 1755, including for example a mutant comprising SEQ ID NO: 11, which corresponds to S1589D/S1755D of the short splice variant; SEQ ID NO: 12, which corresponds to S1589D/S1755D of the long splice variant.

The invention also provides the amino acids of SEQ ID NO: 9, SEQ ID NO: 10, SEQ ID NO:11, SEQ ID NO:12 having one or more conservative amino acid substitutions, wherein the Ca²⁺ release function is maintained. Also provided are mutants comprising amino acid sequences having at least 80, 85, 90, 95, 96, 97, 98, 99% (or any amount in between these values) homology to the amino acid sequence of SEQ ID NO:9, 10, 11, or 12, wherein the Ca²⁺ release function is maintained. Also provided are the comparable S1589E/S1755E and S1589D/S1755D mutants for various species.

The invention further provides double mutants, wherein aspartate is substituted for serine at residue 1589 and glutamate is substituted for serine at residue 1755. For example, the invention provides a mutant comprising the amino acid sequence of SEQ ID NO: 13, which corresponds to S1589D/S1755E short splice variant; and SEQ ID NO:14, which corresponds to a S1589D/S1755E mutation in the long splice variant. The invention also provides a mutant, wherein glutamate is substituted for serine at residue 1589 and aspartate is substituted for serine at residue 1755; including, for example, a mutant comprising the amino acid of SEQ ID NO: 15, which corresponds to a S1589E/S1755D mutant of the short splice variant; and a mutant comprising the amino acid of SEQ ID NO: 16, which corresponds to a S1589E/S1755D mutation in the long splice variant.

The invention also provides the amino acids of SEQ ID NO: 13, 14, 15, 16, having one or more conservative amino acid substitutions, wherein the Ca²⁺ release function is maintained. Also provided are mutants comprising amino acid sequences having at least 80, 85, 90, 95, 96, 97, 98, 99% (or any amount in between these values) homology to the amino acid sequence of SEQ ID NO:13, 14, 15, or 16, wherein the Ca²⁺ release function is maintained. Also provided are the comparable S1589D/S1755E and S1589E/S1755D mutants for various species.

Similar to those outlined above for the InsP₃R-1 mutant, the invention provides phosphomimetic InsP₃R-2 mutants. More specifically, the mutant comprises at least one substitution of serine with a negatively charged amino acid residue at a phosphorylation site, wherein the phosphorylation site is selected from residues 766, 1772, 1856, 1772, 1856, 2058, or 2227. For example, the sequence of SEQ ID NO: 19 can be modified to form S766E, S766D, S1772D, S1772E, S1856E, S1856D, S2058E, S2058D, S2227E, S2227D. Furthermore, any combination of these serine substations can be made to form mutants with two, three, four, or five different substitutions. As for the InsP₃R-1 mutants, the invention further provides substitution InsP₃R-1 mutants wherein the amino acid sequence modified from the wild type sequence, provided herein as SEQ ID NO: 19, has one or more conservative amino acids, wherein the Ca²⁺ release function is maintained. Also provided are mutants comprising amino acid sequences having at least 80, 85, 90, 95, 96, 97, 98, 99% (or any amount in between these values) homology to the amino acid sequence modified from the wild type sequence (provided herein as SEQ ID NO:19), wherein the Ca²⁺ release function is maintained. Also provided are the comparable InsP₃R-2 mutants for various species designed by aligning the InsP₃R-2 of the species with the rat species an substituting the corresponding amino acid sequence.

Similar to those outlined above for the InsP₃R-1 mutant, the invention provides phosphomimetic InsP₃R-3 mutants. More specifically, the mutant comprises at least one substitution of serine with a negatively charged amino acid residue at a phosphorylation site, wherein the phosphorylation site is selected from residues 934, 1640, 1834, 2009, 2041, or 2189. For example, the sequence of SEQ ID NO:20 can be modified to form S934E, S934D, S1640D, S1640E, S1834E, S1834D, S2009E, S2009D, S2041E, S2041D, S2189E, or S2189D. Furthermore, any combination of these serine substitutions can be made to form mutants with two, three, four, five, or six different substitutions. As for the InsP₃R-1 mutants, the invention further provides substitution InsP₃R-1 mutants wherein the amino acid sequence modified from the wild type sequence, provided herein as SEQ ID NO:20, has one or more conservative amino acids, wherein the Ca²⁺ release function is maintained. Also provided are mutants comprising amino acid sequences having at least 80, 85, 90, 95, 96, 97, 98, 99% (or any amount in between these values) homology to the amino acid sequence modified from the wild type sequence (provided herein as SEQ ID NO:20), wherein the Ca²⁺ release function is maintained. Also provided are the comparable InsP₃R-2 mutants for various species designed by aligning the InsP₃R-2 of the species with the rat species and substituting the corresponding amino acid sequence.

The invention also provides mutants that are nonphosphorylatable. Specifically, the invention provides an InsP₃R mutant comprising at least one substitution of serine with an amino acid with an aliphatic side chain at a phosphorylation site of a wild-type InsP₃R, wherein the mutant is nonphosphorylatable. Preferably, the amino acid with the aliphatic side chain is alanine. The mutant can be a modified InsP₃R-1 (either the long or short splice variant), InsP₃R-2, or InsP₃R-3. Thus, the sequence of SEQ ID NO: 17 can be modified at residues 1589 and 1775 to form a null mutant, SEQ ID NO:18 can be modified at S1755 to form a null mutant. Similarly, nonphosphorylatable mutants of InsP₃R-2 can be formed by substituting alanine for any one or more of the residues 766, 1772, 1856, 2058, 2227 of a wild-type InsP₃R-2 sequence or any combination thereof. Nonphosphorylatable mutants of InsP₃R-3 can be formed by substituting alanine for any one or more of the residues 934, 1640, 1834, 2009, 2041, 2189 of a wild-type InsP₃R-3 sequence or any combination thereof. Similar mutants in various species can be derived by aligning the sequence with the rat sequence provided herein and making the null-inducing substitutions provided herein in the corresponding serine residue.

It is understood and herein contemplated that the short-form splice variants of an InsP₃R comprise a unique binding motif GXGXXG (SEQ ID NO: 24) formed by the deletion of 40 amino acids. Thus, it is understood that the short form splice variant can be a variant of InsP₃R-1, InsP₃R-2, or InsP₃R-3. It is also understood and herein contemplated that the short-form splice variants formed by this deletion (e.g., the short form variant InsP₃R-1 S2− (SEQ ID NO: 17)) can comprise substitutions one or more of the amino acids that form the binding site. For example, one or more of the glycines at the unique binding site can be substituted with an alanine. Thus, for example, specifically contemplated are InsP₃R-1 S2− variant comprising a substitution of a glycine with an alanine at position 1690 (SEQ ID NO: 23). It is understood an herein contemplated that any of the glycines can be substituted with an alanine as described herein.

As discussed herein there are numerous variants of the InsP₃R protein that are known and herein contemplated. In addition, to the known functional InsP₃R strain variants, there are derivatives and fragments of the InsP₃R proteins that also function in the disclosed methods and compositions. Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Table 1 and are referred to as conservative substitutions.

TABLE 1
Amino Acid Substitutions Original Residue
Exemplary Conservative Substitutions, others are known in the art.
Ala; Ser
Arg; Lys; Gln
Asn; Gln; His
Asp; Glu
Cys; Ser
Gln; Asn, Lys
Glu; Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or 0-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular sequence of the “short form” (S2⁻) of InsP₃R-1 (short form S 1589E) and SEQ ID NO: 2 sets forth a particular sequence of the “long form” (S2⁺) of InsP₃R-1 protein (long form S1589E). Specifically disclosed are variants of these and other proteins herein disclosed which have at least, 70% or 75% or 80% or 85% or 90% or 95% homology to the stated sequence. Those of skill in the art readily understand how to determine the homology of two proteins.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 18 is set forth in SEQ ID NO: 22. In addition, for example, a disclosed conservative derivative of SEQ ID NO: 1 is shown in SEQ ID NO: 3, where the isoleucine (E) at position 1589 is changed to a valine (D). It is understood that for this mutation all of the nucleic acid sequences that encode this particular derivative of the short form S1589E are also disclosed. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

Also provided are fragments of the proteins described below, wherein the fragments maintain the Ca²⁺ enhancing or reducing function of full length protein. Preferably the fragment will have at least 50% of the enhancing function of the full length correlate or at least a 50% reduction of the Ca²⁺ reducing function.

### Nucleic Acids, Vectors and Expression Systems

The invention further provides nucleic acids that encode the mutants described herein. Examples of nucleic acids that encode the InsP₃R-1 wild type receptors can be found at http://www.ncbi.nlm.nih.gov/HomoloGene/homol.cgi?HID=30927, including for example rat (ATCC Acc. No. xm_{—}342732); mouse (ATCC Acc. No. nm_{—}010585); human (ATCC Acc. No. nm_{—}002222). Examples of nucleic acids that encode the InsP₃R-2 wild-type receptors can also be found at the http://www.ncbi.nlm.nih.gov site and include, for example, rat (ATCC Acc. No. NM_{—}031046), human (ATCC Acc. No. NM_{—}002223). Examples of nucleic acids that encode the InsP₃R-3 wild-type receptors can also be found at the http://www.ncbi.nlm.nih.gov site and include, for example, rat (ATCC Acc. No. NM_{—}013138), human (ATCC Acc. No. NM_{—}002224), and mouse (ATCC Acc. No. NM_{—}080553).

One of skill in the art could modify these nucleic acid sequences to make the substitutions described herein. For example, the codon for the selected serine is replaced by a codon for glutamate, aspartate, or alanine. Further provided are nucleic acids that comprise a sequence that hybridizes under highly stringent conditions to the various rat, mouse, and human mutatnts encoding nucleic acids with the selected serine(s) substituted with glutamate, aspartate, or alanine. Preferably these hybridizing nucleic acids do not hybridize to the wild-type encoding nucleic acids.

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent or highly stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

Also provided are fragments of at least 10, 20, 30, 40, 50, 60, 70, 80, 90, 100 nucleotides (or any number in between) of the nucleic acids provided herein, wherein the fragment encodes a serine substitution described herein.

The invention also provides an expression vector comprising a nucleic acid of the invention wherein the nucleic acid is operable linked to an expression control sequence.

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modifed to specifically function with large DNA molecules. Further, these methods can be used to target certain diseases and cell populations by using the targeting characteristics of the carrier.

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as SEQ ID NO: 22 into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang, BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993) ; Zabner, Nature Genetics 6:75-83 (1994); Guzman, Circulation Research 73:1201-1207 (1993) ; Bout, Human Gene Therapy 5:3-10 (1994); Zabner, Cell 75:207-216 (1993) ; Caillaud, Eur. J. Neuroscience 5:1287-1291 (1993) ; and Ragot, J. Gen. Virology 74:501-507 (1993) ). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B 19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Molecular genetic experiments with large human herpesviruses have provided a means whereby large heterologous DNA fragments can be cloned, propagated and established in cells permissive for infection with herpesviruses (Sun et al., Nature Genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

The disclosed compositions can be delivered to the target cells in a variety of ways. For example, the compositions can be delivered through use of a gene gun, electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors for example, lipids such as liposomes (cationic liposomes (e.g., DOTMA, DOPE, DC-cholesterol) or anionic liposomes). Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993) ; Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other speciifc cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

The invention also provides a cell comprising a vector described herein. Preferably the cultured cell, in the absence of the vector does not express InsP₃Rs. Optionally, the cell is a cultured cell. An example of such a cell is the DT-40 3ko cell. Optionally, the cell further comprises a nucleic acid that encodes an acetylcholine receptor (including for example an M3 receptor). Optionally various labels can be used to identify the cells that are successfully transfected or transformed.

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

The viral vectors can include nucleic acid sequence encoding a marker product. This marker product is used to determine if the gene has been delivered to the cell and once delivered is being expressed. Preferred marker genes are the E. coli lacZ gene, which encodes B3-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

The invention further provides methods of using the mutants, nucleic acids, and cells of the invention in various methods of making the mutant receptors, methods of screening for agents that modulate the InsP₃Rs, and methods of treatment.

The expression systems described herein can be used to make mutant InsP₃Rs. Thus, the invention provides making mutant InsP₃R-1, InsP₃R-2, InsP₃R-3 or fragments thereof described herein by culturing a cell that expresses the selected mutant or fragments under conditions that allow expression and by isolating the expressed mutant or fragment thereof.

The invention further provides a method of screening for an agent that preferentially modulates Ca²⁺ release by phosphorylated InsP₃R, comprising contacting a cell of the invention with the agent to be screened, under conditions that allow Ca²⁺ release; measuring Ca²⁺ release; and comparing the amount of Ca²⁺ release with a control cell. The control cell can comprise an unphosphorylated InsP₃R that is not contacted with the agent to be screened. Conditions that allow calcium release include for example carbachol, acting at muscaric M3 receptors or alternatively, an agonist acting at any one of over one hundered plasma membrane receptors for neurotransmitters, hormones and growth factors coupled to the formation of InsP₃. In addition, ehanced Ca²⁺ release can occur following direct activation of InsP₃R with InSP₃ or its analogs. An increase or decrease in Ca²⁺ release as compared to a control cell indicates an agent that preferentially modulates unphosphorylated InsP₃R. By “modulation” is meant any physiologic effect that increases or decreases InsP₃R stimulated calcium release. Preferably the unphosphorylated InsP₃R is a nonphosphorylatable mutant InsP₃R described herein.

The invention also provides a method of expressing a mutant InsP3R in a cell in vivo, comprising providing an expression vector described herein; introducing the vector into a cell in vivo; maintaining the cell under conditions that permit expression of the mutant InsP₃R by the cell.

Also provided herein are methods of treating a subject with reduced Ca²⁺ release, comprising introducing into the subject an expression vector that encodes a phosphomimetic mutant of the invention, under conditions that an amount of the mutant receptor is expressed in an effective amount to enhance Ca²⁺ release. Examples of such diseases include, xerostomia, cystic fibrosis, or a large class of diseases which result from the decreased secretion of hormones, these include but are not limited to, decreased thyroid stimulating hormone (TSH) secretion from the pituitary or insulin secretion from the pancreas. The former deficiency results in dwarfism and cretinism, the latter diabetes.

Optionally the expression vector can be targeted. For example, to treat a subject with a condition like xerostomia, the vector could be targeted to the oral mucosal cells.

The invention also provides a method of treating a subject with cystic fibrosis, comprising introducing into the subject the expression vector of that expresses a phosphomimetic mutant receptor under conditions that an amount of the mutant receptor is expressed in an effective amount to alleviate the symptoms of cystic fibrosis.

It is understood and herein contemplated that the methods of treatment disclosed herein can also be used for the treatment of HIV and arthritis. Thus for example, specifically contemplated are methods of treating a subject with HIV comprising introducing into the subject the expression vector of the invention under conditions that an amount of InsP₃R mutant is expressed in an effective amount to alleviate the symptoms of HIV. Also disclosed are methods of treating a subject with arthritis comprising introducing into the subject the expression vector of the invention under conditions that an amount of InsP₃R mutant is expressed in an effective amount to alleviate the symptoms of arthritis.

Additional treatment methods are also contemplated. For example, the disclosed constructs can be used to inhibit apoptosis in a transplant. Thus, herein contemplated are methods of reducing apoptosis of cells of a transplant comprising introducing into the transplant a vector comprising one of the mutants disclosed herein. For example, the apoptosis of B cells being transplanted into a subject can be inhibited by introducing an InsP₃R mutant into the B cells ex-vivo prior to administering the same cells to a subject. It is understood and herein contemplated that “inhibit” or “inhibition” can refer to any treatment that results in any reduction of an event relative to an untreated condition. Thus, it is understood that inhibition can include a 5% reduction in the occurrence or presence of a condition or symptom as well as the complete ablation of an occurrence or presence of a condition or symptom. Thus, for example, inhibition of apoptosis can include a 5% reduction in the occurrence of cell death in a given cell population as well as the complete ablation of apoptosis related cell death in the same population.

### EXAMPLES

### Example 1

### Phosphorylation of Type-1 Inositol 1,4,5-Trisphosphate Receptors by Cyclic Nucleotide-Dependent Protein Kinases

Inositol 1,4,5-trisphosphate receptors (InsP₃R) constitute the major route of intracellular calcium release in eukaryotic cells and as such are pivotal for stimulation of Ca²⁺ dependent effectors important for numerous physiological processes. Modulation of this release has important consequences for defining the particular spatio-temporal characteristics of Ca²⁺ signals. In this study, regulation of Ca²⁺ release by phosphorylation of type-1 InsP₃R (InsP₃R-1) by cAMP (PKA) and cGMP (PKG) dependent protein kinases was investigated in the two major splice variants of InsP₃R-1. InsP₃R-1 were expressed in DT-40 cells devoid of endogenous InsP₃R. In cells expressing the neuronal, S2⁺ splice variant of the InsP₃R-1, Ca²⁺ release was markedly enhanced when either PKA or PKG was activated. The sites of phosphorylation were investigated by mutation of serine residues present in two canonical phosphorylation sites present in the protein. Potentiated Ca²⁺ release was abolished when serine 1755 was mutated to alanine (S1755A) but was unaffected by a similar mutation of serine 1589(S1589A). These data demonstrate that S1755 is the functionally important residue for phosphoregulation by PKA and PKG in the neuronal variant of the InsP₃R-1. Activation of PKA also resulted in potentiated Ca²⁺ release in cells expressing the non-neuronal, S2⁻ splice variant of the InsP₃R-1. However, the PKA-induced potentiation was still evident in S1589A or S1755A InsP₃R-1 mutants. The effect was abolished in the double (S1589A/S1755A) mutant, indicating both sites are phosphorylated and contribute to the functional effect. Activation of PKG had no effect on Ca²⁺ release in cells expressing the S2⁻ variant of InsP₃R-1. Collectively these data indicated that phosphoregulation of InsP₃R-1 had dramatic effects on Ca²⁺ release and defined the molecular sites phosphorylated in the major variants expressed in neuronal and peripheral tissues.

Most studies of PKA-dependent phosphorylation have been performed on the S2⁺ neuronal type-I InsP₃R, the so called “long-form” of the receptor. In this variant of the InsP₃R-1, serine residues at S1589 and S1755 are phosphorylated by PKA [7, 34, 35], with S1755 being more heavily phosphorylated [34]. In contrast, little consensus exists as to the effect of PKG; in situ experiments in cerebellar slices reported S1589 to be preferentially phosphorylated by PKG [35], while other studies suggest that purified InsP₃R-1 protein from the cerebellum was phosphorylated preferentially on S1755 [36].

Alternative splicing of the type-1 receptor gene results in the S2⁻ variant of the type-1 InsP₃R where 40 amino acids (amino acids 1693 to 1732) are excised between the two phosphorylation sites [6, 7]. This protein is predominantly expressed in peripheral tissues and interestingly has been reported to be exclusively phosphorylated on S1589 by PKA [7], but on S1755 by PKG [36]. Studies of the functional effects of phosphorylation of the peripheral form have suggested that in contrast to the neuronal form of the receptor, phosphorylation of the “short form” of the InsP₃R-1 results in attenuated Ca²⁺ release [21, 22]. Thus, prior to this invention, the possibility existed that differences in both the sites of phosphorylation and therefore the functional effects of phosphorylation were defined by the particular splice variants expressed in particular tissues.

In this study the sites of phosphorylation by PKA and PKG, functionally important for regulation of Ca²⁺ release in the two major splice variants of the InsP₃R-1, were investigated. By expression of mutant InsP₃R-1 in InsP₃R null DT-40 cells [42, 43], the studies revealed that phosphorylation of S1755 by PKA or PKG resulted in markedly enhanced Ca²⁺ release for S2^{+ InsP}₃R-1. Notably, in S2⁻ InsP₃R expressing cells PKG activation did not markedly alter Ca²⁺ release while PKA phosphorylation of both S1755 and S1589 result in enhanced Ca²⁺ release. Thus, the expression of particular InsP₃R splice variants defined the functional consequences of phosphoregulation by cyclic nucleotide-dependent kinases.

### Materials:

The acetoxymethylesters of Fura-2, and Fluo-4 were purchased from Molecular Probes (Eugene, Oreg.). Fura-2FF was purchased from Tefabs (Austin, Tex.). Cell permeable cyclic nucleotides and forskolin were purchased from Biomol, (Plymouth Meeting, Pa.). All other chemicals were purchased from Sigma Chemical Company (St. Louis, Mo.). The DT-40 cells lacking InsP₃R (DT-40 3ko) were kindly provided by Dr Kurosaki, (Kansai Medical University, Japan) and were maintained as previously described [42-44].

### Production of Mutations:

The S2^{+ InsP}₃R-1 in the expression plasmid pIRES-GFP (Clontech; Palo Alto, Calif.) was digested with the restriction endonuclease Sal I. The overhang created by digestion was blunted using T4 polymerase. An EcoR I linker was then ligated onto the blunted ends of the construct. The entire receptor DNA was excised from the plasmid using EcoR I and ligated into the plasmid MXT-1. The region containing the S2⁻ splice variant and potential PKA phosphorylation sites was excised from its backbone in pCDNA 3.1+ (Invitrogen; Carlsbad, Calif.) by Rsr II and Kas I and ligated into the InsP₃R construct in MXT-1. The potential PKA sites, S1589 and S1755, were mutated, individually in both splice variants and together in the S2⁻ splice variant, to alanines using sequential PCR mutagenesis. The outer oligos used for the mutagenesis reaction flanked the restriction sites Rsr II and Kas I. Following mutation, the resulting fragments were cut with Rsr II and Kas I and inserted into the IP₃R-1 backbone at the corresponding sites. The mutations and lack of spurious misincorporations were confirmed by Big Dye fluorescent sequencing. Mutated receptor DNAs were excised from MXT-1 using EcoR I and ligated into the mammalian expression vector pGW (British Biotechnology; Oxford, UK). Orientation was confirmed using restriction enzyme digestion.

### Transfection of DT-40 cells:

DT-40 cells lacking all three InsP₃ receptor subtypes DT-40 3ko were transfected using electroporation, 350 V and 950 μF. 2×10⁷ cells were co-transfected with 25 μg of the InsP₃R cDNA, 25 μg of the muscarinic type 3 (M3) receptor, and 4 μg of the red fluorescent protein plasmid pHcRed1-N1. Cells were incubated with DNA in 500 μL of Optimem media on ice for 10 minutes. The cell/DNA mixture was electroporated, incubated on ice for 30 minutes, brought up to 5 ml with Optimem and placed in a 5% C0₂ incubator at 39° C. for 5 hours. The cells were then centrifuged and resuspended in 12 ml of complete RPMI media. Transfection efficiency was typically ˜20%. Experiments were performed within 32 hours of transfection.

### Digital Imaging of [Ca⁺²]ᵢ:

Transfected DT-40 3ko cells were washed once in a HEPES-buffered physiological saline solution (HEPES-PSS) containing (in mM) 5.5 glucose, 137 NaCl, 0.56 MgCl₂, 4.7 KCl, 1 Na₂HPO₄, 10 HEPES (pH 7.4), 1.2 CaCl₂ and 1% w/v Bovine Serum Albumin. Cells were then resuspended in BSA HEPES-PSS with 1 μM Fura-2 (AM), placed on a 15 mm glass coverslip in a low volume perfusion chamber (Warner Instruments) and allowed to adhere for 30 minutes at room temperature. Cells were perfused continually for 10 minutes with HEPES-PSS before experimentation to allow Fura-2 de-esterification. A field of cells for each experiment was chosen that contained a wide range of transfection efficiency based upon the intensity of red fluorescence emitted when excited at 560 nm. [Ca²⁺]ᵢ imaging was performed essentially as previously described [28, 29, 41] using an inverted epifluorescence Nikon microscope with a 40× oil immersion objective lens (numerical aperture, 1.3). Cells were excited alternately with light at 340 and 380 nm (±10 mn bandpass filters, Chroma) using a monochrometer (TILL Photonics). Fluorescence images were captured and digitized with a digital camera driven by TILL Photonics software. Images were captured every 2 seconds with an exposure of 35 ms and no binning. 340/380 ratio images were calculated online and stored immediately to hard disk. Only data from cells exhibiting an increase in ratio units of less than 0.2 upon stimulation were used for further analysis.

### Flash Photolysis:

Transfected cells were simultaneously loaded by incubation with the visible wavelength indicator Fluo-4 and a cell permeable form of caged-inositol trisphosphate (ciIP₃/PM) for 30 min. Ci-IP₃/PM is a homologue of cm-IP₃/PM [45] The 2- and 3-hydroxyls of the inositol ring were protected by an isopropylidene group in ci-IP3/PM, and were protected by a methoxymethylene group in cm-IP₃/PM. Like cm-IP3/PM, ci-IP3/PM diffuses across cell membranes and induces internal calcium release upon photo-uncaging. A further period of approximately 30 min was allowed for de-esterification of both dye and cage. Cells were illuminated at 488±10 nm and fluorescence collected through a 525±25 nm band pass filter and captured using the Till Photonics imaging suite. These traces are displayed as % ΔF/F₀, where F is the recorded fluorescence and F₀ is the mean of the initial 10 sequential frames. Photolytic release was performed as previously described [28, 29, 41] using a pulsed Xenon arc lamp (Till Photonics). A high intensity (0.5-5 msec duration; 80 J) discharge of UV light (360±7.5 nm) was reflected onto the plane of focus using a DM400 dichroic mirror and Nikon×40 oil immersion objective, 1.3 NA.

### Statistical Analysis:

The effects of treatment were determined by normalizing the peak change in fluorescence ratio by stimulation following forskolin or 8-Br cGMP exposure to that of stimulation in control HEPES-PSS. Thus, pooled data represents a normalized fold increase over control for the treated trial. Two tailed heteroscedastic t-tests with P values<0.05 were considered to have statistical significance.

### PKA phosphorylation of S1755 in S2⁺ InsP₃R-1 resulted in enhanced Ca²⁺ release:

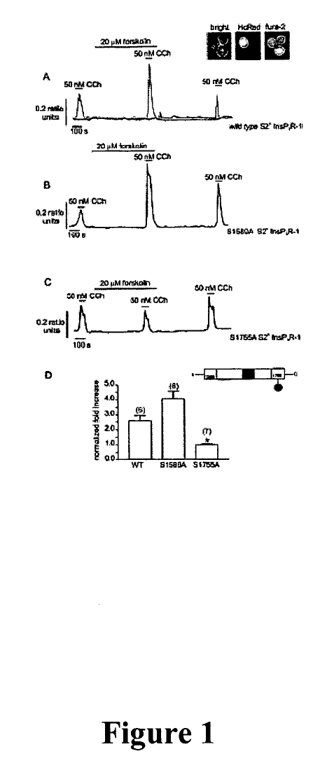

Experiments were performed in DT-40 3ko cells transfected with S2⁺ InsP₃R-1. Since there are no reports of Gαq coupled receptors expressed in DT-40 cells, initial experiments were performed eliciting [Ca²⁺]ᵢ changes by stimulating the endogenous B cell receptor with α-IgM antibody. This resulted in somewhat irregular Ca²⁺ oscillations, which were not reversible when the antibody was removed making paired analysis of any effects of raising cyclic nucleotides difficult to interpret. Thus, in all further experiments, DT-40 3ko cells were co-transfected with the M3 receptor, and HcRed to facilitate identification of transfected cells (inset FIG. 1). Stimulation of M3 receptors with the muscarinic agonist carbachol (CCh), provided a convenient means of stimulating [Ca²⁺]ᵢ changes, presumably through Gαq-induced activation of phospholipase C and production of InsP₃. Stimulation with a low concentration of CCh (25-50 nM) resulted in an increase in [Ca²⁺]ᵢ, which returned to baseline when the agonist was removed. Following 10 min incubation with 20 μM forskolin to maximally raise cAMP, the initial peak in [Ca²⁺]ᵢ elicited by identical CCh treatment was markedly potentiated (FIG. 1A and pooled data in FIG. 1D). After washout of forskolin a subsequent stimulation resulted in a [Ca²⁺]ᵢ change comparable to the initial exposure. The potentiation was most marked when threshold elevations in [Ca²⁺]ᵢ were evoked by the initial exposure to CCh and therefore only cells in which the initial CCh treatment evoked a Δ 340/380 ratio of<0.2 ratio units were included for analysis. These data showed that phosphorylation of the S2⁺ InsP₃R resulted in enhanced Ca²⁺ release by increasing the sensitivity of the receptor to InsP₃. [Ca²⁺]ᵢ changes were never evoked by CCh treatment in cells transfected with only M3 cDNA in the absence of InsP₃R or likewise InsP₃R with no M3 cDNA. Similarly, cells exhibiting no HcRed fluorescence seldom responded to CCh treatment (black trace, FIG. 1A).

To ascertain whether one or both of the serine residues S1589 or S1755 is important for phosphoregulation of the S2⁺ variant of the InsP₃R-1 following PKA activation, individual point mutations were constructed where these serine residues were mutated to alanine (S1589A and S1755A). A similar potentiation of the initial peak of the CCh-induced [Ca²⁺]ᵢ elevation was observed following forskolin treatment in cells expressing S1589A S2⁺ InsP₃R (FIG. 1B and 1D), however mutation of S1755A resulted in the complete abrogation of any potentiation upon forskolin treatment (FIG. 1C and 1D). These data clearly support the assertion that phosphorylation of S1755 is the important event underlying enhanced Ca²⁺ release through the neuronal InsP₃R-1 following PKA activation. It follows therefore, that phosphorylation of S1589 after 20 μM forskolin treatment (which could reasonably be expected to result in the maximal generation of cAMP) was either not occurring to a significant extent, or perhaps more likely, was functionally not important in modulating Ca²⁺ release.

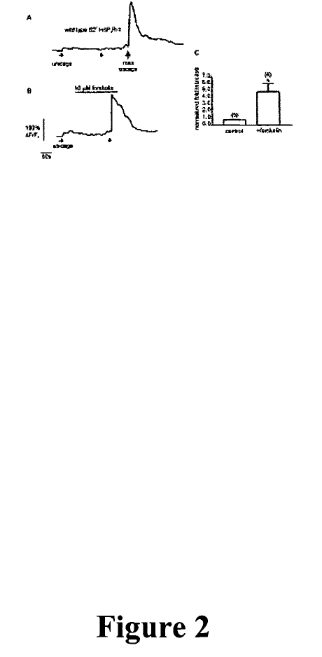

Since the only difference between the experiment in FIG. 1A and 1C is a conservative point mutation in the InsP₃R-1, it was assumed that the effects observed on the peak Ca²⁺ signal following forskolin treatment were predominately the results of alteration of Ca²⁺ release. To rule out the possibility that phosphorylation by PKA of other signaling molecules caused the effect, experiments were performed utilizing ciIP₃/PM, a cell permeable form of caged InsP₃ [45] to more directly induce Ca²⁺ release in cells transfected with wild-type S2⁺ InsP₃R-1. Using a brief flash of UV light (˜0.5 ms, indicated by the arrow in FIG. 2) small elevations in [Ca²⁺]ᵢ could be evoked. Subsequent exposure to UV light never produced an increase larger than that initially evoked, however longer flashes of UV light (5 msec) could evoke larger peak increases (FIG. 2A; max uncage, indicated by the large arrow-head). No effect of UV light was observed in cells either not loaded with cage, or alternatively not expressing InsP₃R. In contrast, when cells were incubated with 10 μM forskolin for 5 min prior to a second identical exposure to UV light a marked increase iii the initial Ca²⁺ peak was evoked (FIG. 2B and pooled data FIG. 2C). This potentiation of InSP₃-induced release was of similar magnitude to that seen for CCh-treated cells exposed to forskolin and supports the notion that the predominant effect of forskolin treatment is to regulate Ca²⁺ release through phosphorylation of InsP₃R.

### PKA -Induced Phosphorylation of S1589and S1755 were Functionally Important in the S2⁻ InsP₃R-1:

The S2⁻ variant of the InsP₃R-1 is predominantly expressed in peripheral tissues and in fetal brain during neuronal development [6, 7]. The S2⁻ InsP₃R-1 has been reported to be phosphorylated by PKA in a number of tissues, including platelets, vas deferens, smooth muscle and hepatocytes [7, 30, 46]. In contrast to the neuronal form of the InsP₃R-1, studies performed with S2⁻ InsP₃R-1 purified from vas deferens and smooth muscle have demonstrated that the receptor is almost exclusively phosphorylated on S1589 [7]. Reports have also suggested a different functional outcome as a result of PKA activation, since the majority of studies, for example in megakaryocytes have suggested that phosphorylation results in inhibition of Ca²⁺ release [22]. An important caveat relevant to the interpretation of this functional data, is that in contrast to InsP₃R-1 in cerebellum (>99% S2⁺ InsP₃R-1), in peripheral tissues multiple InsP₃R types are invariably expressed to varying degrees [47]. Thus, unequivocally attributing an effect to an individual homotetrameric receptor was problematic prior to the invention.

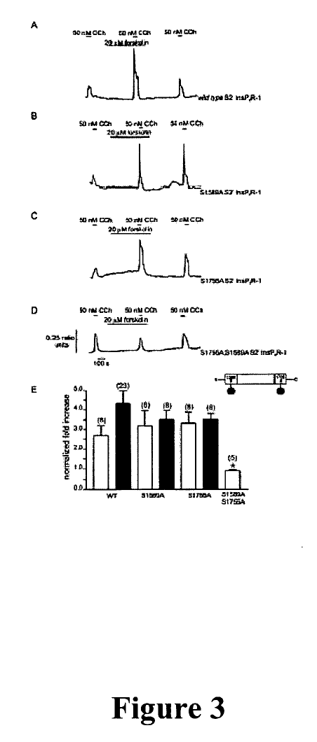

Similar experimental paradigms were employed to assess the effect of PKA-induced phosphorylation on a homogeneous population of S2⁻ InsP₃R-1. In DT-40 3ko cells expressing wild-type S2⁻ InsP₃R-1, incubation with 20 μM forskolin for 10 min resulted in a marked potentiation of the initial CCh-induced Ca²⁺ peak (FIG. 3A, pooled data FIG. 3E) in a similar fashion to that demonstrated for the S2⁺ InsP₃R-1. In contrast to the S2⁺ variant of the InsP₃R-1, neither single mutation in amino acids corresponding to S1589 or S1755 in S2⁺ InsP₃R-1 resulted in loss of this enhanced Ca²⁺ signal (FIGS. 3C/3D and 3E). These data indicated that both serine residues can be phosphorylated in this form of the receptor and furthermore was consistent with the observation that the peripheral InsP₃R-1 is more readily phosphorylated by PKA than the neuronal form [7]. However, no potentiation was observed in cells transfected with a double mutant where both serines were mutated to alanine (S1589A/S1755A S2⁻ InsP₃R-1), confirming that no additional functionally important phosphorylation sites are present in the S2⁻ InsP₃R-1 (FIGS. 3D and 3E).

The degree of potentiation appeared similar when comparing wild-type to either S1589A or S1755A S2⁻ InsP₃R-1; expression of each construct revealed a ˜3 fold increase in CCh-induced Ca²⁺ release in the presence of forskolin. These data indicated, that if the assumption is made that phosphorylation of each particular site occurred independently, that phosphorylation of individual sites appeared not to result in an additive effect on Ca²⁺ release. A potential exists, however, that the dye used in these experiments (Fura-2; kd ˜150 nM) is saturated with Ca²⁺ upon CCh exposure in the presence of forskolin, thereby masking any additive effect of phosphorylating both sites. For this reason, similar experiments were performed using the lower affinity Ca²⁺ indicator Fura-2-FF (kd ˜10 μM). While the degree of potentiation was somewhat greater in cells expressing wild-type S2⁻ InsP₃R-1 (˜4.5 fold) there was no significant difference in the extent of enhancement when comparing wild-type to S1589A and S1755A expressing cells (FIG. 3E; filled bars). These data indicate that while it is possible that Fura-2 measurements may indeed underestimate the degree of PKA-induced enhancement of Ca²⁺ release resulting from PKA phosphorylation, our data suggests that phosphorylation of individual sites appears not to be functionally additive.

To assess if the phosphorylation of a particular site was favored in S2⁻ InsP₃R-1, the minimal concentration of forskolin sufficient to enhance CCh-induced Ca²⁺ release in wild-type vs. S1589A and S1755A mutants was compared. In cells transfected with wild-type S2⁻ InsP₃R-1, 100 nM, 500 nM or 1 μM forskolin failed to enhance the subsequent CCh-induced Ca²⁺ release (3 experiments, >5 cells for each condition). Incubation with 5 μM forskolin resulted in a 1.26±0.21 fold potentiation of Ca²⁺ release (n=6). At this threshold concentration of forskolin a similar degree of potentiation was observed in both S1589A (1.36±25 fold; n=8) and S1755A (1.37±0.26 fold; n=7) S2⁻ InsP₃R-1. Thus, functionally, it appears that a particular site is not obviously subject to preferential phosphoregulation by PKA.

These data demonstrated that, in contrast to the S2⁺ variant, both S1589 and S1755 are functionally important phosphorylation substrates in the S2⁻ InsP₃R. This indicates that excision of the 40 amino acids in the S2⁻ form of the InsP₃R-1 either alters the structure of the receptor allowing access to the kinase or perhaps allows the interaction with an accessory protein necessary to confer this effect. The only known structural difference between the splice variants is the insertion of an adenine nucleotide binding site in the S2⁻InsP₃R-1.

### Phosphorylation of S2⁺ InsP₃R-1 by PKG:

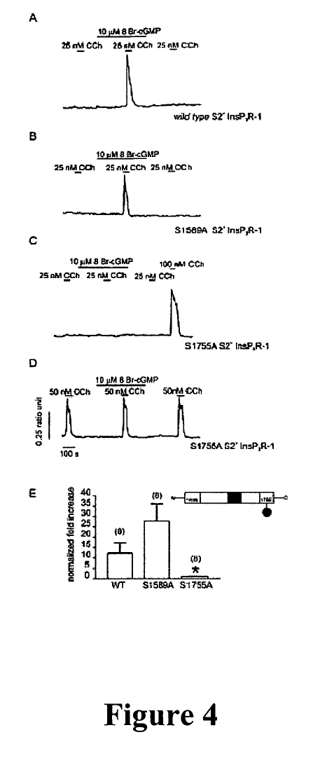

Similar experiments were performed to assess the effects on Ca²⁺ release of phosphorylating InsP₃R-1 with PKG. Cells transfected with S2^{+ InsP}₃R-1 together with M3 receptor and HcRed were stimulated with low, threshold concentrations of CCh (25-50 nM) followed by a 10 min treatment with 10 μM 8-Br cGMP, a specific activator of PKG [48]. Subsequent re-stimulation with an identical concentration of CCh revealed a marked potentiation of the [Ca²⁺]ᵢ change. In a similar fashion to PKA activation, this was manifested as an increase in the CCh-induced initial peak (FIG. 4A and pooled data FIG. 4E). Moreover, these data are again consistent with phosphorylation increasing the sensitivity of the InsP₃R to InsP₃, since sub-threshold increases in [Ca²⁺]ᵢ were readily potentiated to substantial increases in [Ca²⁺]ᵢ (FIGS. 4A 4B and 4E).

Next, the effect of phosphorylation of S1589 and S1755 in S2⁺ InsP₃R-1 mutants was assessed. In cells transfected with S1589A a similar potentiation by 8-Br cGMP was observed (FIGS. 4B and 4E) while no enhanced [Ca²⁺]ᵢ signal was observed in cells transfected with S1755A (FIGS. 4C and 4E), strongly indicating that phosphorylation of S1755 by PKG is the important event underlying this potentiation of Ca²⁺ signaling. Although PKG and PKA consensus motifs are similar (RRXS) these data are consistent with reports that S1755 is phosphorylated by PKG and furthermore that phosphorylation by PKG is enhanced by the presence of an aromatic amino acid, 4 amino acids downstream from the phosphorylatable serine as is the case for S1755 in InsP₃R-1 [49]. Indeed the substantial degree of potentiation may reflect the favorable nature of this site for phosphorylation by PKG. Although phosphorylation of S1755 appears to be responsible for the potentiated signal, our data thus far does not exclude the possibility that the response of wild-type S2⁺ InsP₃R-1 is a result of a net phosphorylation of both serines by PKG, with phosphorylation of S1589 actually resulting in inhibited release. Thus, the effect of PKG activation on larger [Ca²⁺]ᵢ responses to CCh stimulation was tested in cells transfected with S1589A InsP₃R-1. Using this paradigm no effect of 10 μM 8-Br cGMP was observed (FIG. 4D), confirming that either S1589 is not phosphorylated or has no functional consequence in S2⁺ InsP₃R-1.

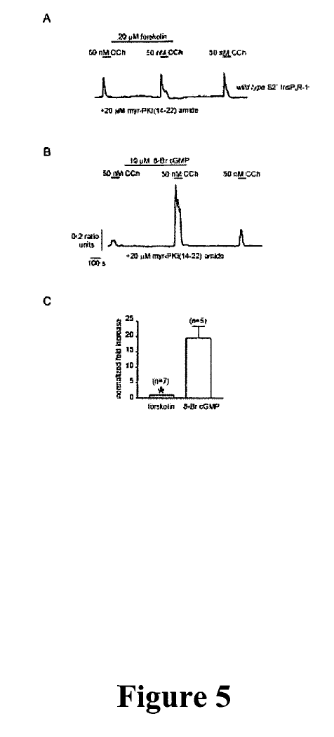

Although the most likely consequence of 8-Br cGMP treatment was to activate PKG, it was possible that this compound led to cAMP accumulation and activation of PKA indirectly by inhibiting cAMP phosphodiesterase [50]. Additionally, a somewhat less likely scenario was that 8-Br cGMP bound to and activated PKA directly. Experiments were therefore performed to confirm that the potentiation of Ca²⁺ signaling observed with 8-Br cGMP treatment was not the result of PKA activation. Cells transfected with S2⁺ InsP₃R-1 were preincubated for 30 minutes with the cell-permeable PKA inhibitor, myristolyated PKI. This treatment completely abolished any potentiation of the CCh-induced [Ca²⁺]ᵢ elevation after forskolin treatment (FIG. 5A and pooled data FIG. 5C). In contrast, similar treatment with PKI did not alter the potentiation induced by 8-Br cGMP treatment (FIG. 5B/C); this treatment still resulted in a 20 fold potentiation of the CCh-stimulated [Ca²⁺]ᵢ signal (compare FIGS. 4A and FIG. 5B). These data clearly indicate that treatment with forskolin and 8-Br cGMP resulted in the selective activation of PKA or PKG respectively.

### Phosphorylation of S2⁻ InsP₃R-1 by PKG:

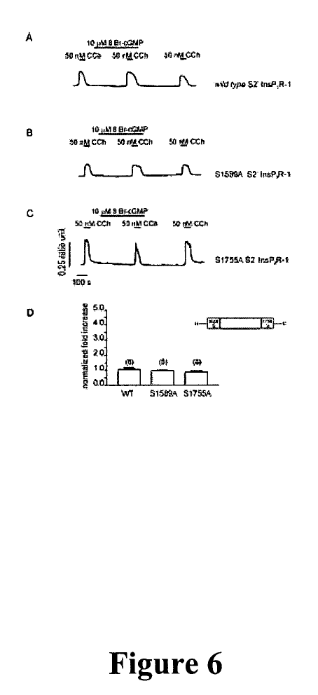

PKA or PKG phosphorylation of the S2⁻ InsP₃R-1 in megakaryocytes and smooth muscle cells has been suggested to inhibit Ca²⁺ release [21, 36, 37]. This observation is difficult to reconcile with the data presented herein for phosphorylation of S2⁻ InsP₃R-1 by PKA, since phosphorylation of either S1589 or S1755 resulted in potentiated release. Nevertheless, similar experiments were performed to elucidate the effect of PKG phosphorylation of S2⁻ InsP₃R-1. Interestingly, no effect of 10 μM 8-Br cGMP treatment was observed in cells transfected with either wild-type (FIG. 6A and pooled data FIG. 6D), S1589A S2⁻ InSP₃R-1 (FIGS. 6B and 6D) or S1755A S2⁻ InsP₃R-1 (FIG. 6C and 6D). However, in the same batches of cells transfected with wild-type S2⁻ InsP₃R-1, treatment with forskolin resulted in the expected ˜4 fold increase in the initial peak (n=5 cells). These data also reinforced the contention that 8-Br cGMP specifically activates PKG without altering PKA activity; the logic being that if, this was not the case, activation of PKA by 8-Br cGMP in S2⁻ InsP₃R-1 expressing cells would be expected to result in data similar to PKA activation shown in FIG. 3.

The data demonstrated that PKA phosphorylation of either S1589 or S1755 resulted in enhanced Ca²⁺ release in the S2⁻ variant of the receptor (FIG. 3 A/B/C). Thus, PKG is simply not capable of directly phosphorylating this receptor. These data, although somewhat surprising, do not rule out the possibility that phosphorylation of an accessory protein termed IRAG [51, 52] may have an effect.

In conclusion, using mutational analysis in a null InsP₃R background this study has elucidated the serine residues in InsP₃R-1 functionally important for modulating Ca²⁺ release by cyclic nucleotide dependent protein kinases. An important finding of this study was that, although phosphorylation of either S1589 or S1755 can result in markedly enhanced Ca²⁺ release, the particular splice variant of InsP₃R-1 expressed dictated which sites were susceptible to phosphorylation. Potentiation of Ca²⁺ release through InsP₃R-1phosphorylation thus provided a powerful means of enhancing and amplifying Ca²⁺ signaling events when multiple signaling pathways are activated. Additionally, the evidence indicated that PKG appears not to directly regulate S2⁻ InsP₃R-1. Therefore, this splice variation defined which kinase is capable of phosphorylating the receptor at these sites and thus the specificity of functional response.

### Example 2

### Acute Regulation of Secretion: Cross-Talk Between Signaling Molecules and Their Effectors

### Effects on InsP₃-Induced Ca²⁺ Release

Phosphorylation of inositol 1,4,5 trisphosphate receptors (InsP₃R) is a major point of synergism between the Ca²⁺ and cAMP signaling systems [29]. Phosphorylation of InsP₃R in parotid acinar cells results in markedly enhanced Ca²⁺ release, an effect attributed to type-II InsP₃R [29]. The functionally important phosphorylation sites have recently been defined in the type-I receptor [57]. The experiments examine the potential for “phosphomimetic” mutants of InsP₃R-1 to enhance Ca²⁺ signaling and the mechanism responsible for this augmentation of Ca²⁺ release in both the InsP₃R-I and InsP₃R-II. These constructs can be utilized in models of impaired fluid secretion to assess their ability to augment fluid secretion.

### Fluid Secretion in Salivary Acinar Cells

Appropriate control of salivary secretion is required for effective speech, mastication and general oral health [62-64]. Disruption of normal secretion is thus a significant health problem for affected individuals. The inability to produce an adequate salivary fluid secretion results in a variety of conditions that together comprise a major health problem for a significant proportion of the population [62, 63].

Fluid secretion in the salivary glands relies on the secondary active transepithelial transport of Cl⁻ [61, 65-68]. Briefly, Cl⁻ ions enter the cell across the basolateral membrane, in part via Cl⁻/HCO₃⁻ exchange but primarily by an electrically neutral Na⁺-K⁺-2Cl⁻ cotransport process [69-72]. The accumulated K⁺ ions recycle to the serosal side via basolateral K⁺ channels, whilst the accumulated Cl⁻ exit to the mucosal side via apical Cl⁻ channels. The resultant transepithelial movement of Cl⁻ generates an electrical potential gradient (lumen negative) sufficient to drive Na⁺ into the lumen via a paracellular pathway. The net result is the secretion of Na⁺ and Cl⁻, with water following osmotically [61, 65, 68]. The principal means of regulation of this fluid secretion involves stimulation via parasympathetic nerves supplying the glands [73]. It is generally acknowledged that the main component of this system involves neurally released acetylcholine (ACh) acting at muscarinic receptors on the acinar cells [74-76]. Activation of these receptors produces a rise in [Ca²⁺]ᵢ as a result of an increased turnover of membrane phosphoinositides and the generation of inositol 1,4,5-trisphosphate (InsP₃) [77]. The elevated [Ca²⁺]ᵢ in turn acts at membrane ion channels, specifically increasing basolateral K⁺ conductance and an apical Cl⁻ conductance [61, 65, 67, 68]. It is the increases in these conductance pathways that lead to the initiation of the secretion of ions and accompanying fluid. The rise in [Ca²⁺]ᵢ also apparently has important additional effects including the stimulation of the activity of the basolateral Na⁺-K⁺-2Cl⁻ cotransport increasing the entry of Cl⁻ ions into the cell [78].

### Ca²⁺ Signaling in Parotid Acinar Cells

Many studies have investigated the characteristics of [Ca²⁺]ᵢ signals in parotid acinar cells [79]. In common with other electrically non-excitable cells, cytosolic Ca²⁺ signals comprise both the release of Ca²⁺ from intracellular stores and the activation of pathways mediating the entry of Ca²⁺ from the extracellular space. It should be noted that while the release of intracellular Ca²⁺ is capable of initiating fluid secretion in the salivary glands, the ability to produce a sustained secretion is known to be entirely dependent on the influx of Ca²⁺ from the extracellular medium [58-60]. Consequently, a thorough understanding of the processes leading to activation and modulation of both Ca²⁺ release and Ca²⁺ influx is fundamentally important for understanding fluid secretion.

The pathways involved in Ca²⁺ release are relatively well defined; the binding of secretagogues such as ACh, substance P, noradrenaline and ATP (acting at P2Y receptors) to plasma membrane receptors, couples to the heterotrimeric G protein Gαq/11, resulting in activation of PLCβ and the subsequent formation of InsP₃. All three isoforms of InsP₃R are expressed to varying degrees in rodent parotid tissue, with ˜80% comprising the type-II (InsP₃R-II) or type-III isoform (InsP₃R-III) and with InsP₃R-II constituting the absolute majority [80, 81]. Binding of InsP₃ to these receptors located in the endoplasmic reticulum results in explosive Ca²⁺ release into the cell cytoplasm. The vast majority of InsP₃R are expressed in the extreme apical pole of the cells and this localization reasonably explains why Ca²⁺ release is initiated in this region [82, 53]. Physiologically, the initial increase in [Ca²⁺]ᵢ is ideally situated to activate Ca²⁺-dependent Cl⁻ channels which are localized exclusively to the luminal membrane [54]. Subsequently, the [Ca²⁺]ᵢ signal rapidly becomes “global”, facilitating the activation of basolateral Ca²⁺ -activated K⁺ channels [80]. The process responsible for the extremely rapid globalization of the Ca²⁺ signal appears to be distinct from other exocrine cells, such as pancreatic acinar cells [80], in that the increase is much too rapid to be mediated by a classical Ca²⁺ wave propagated by sequentially Ca²⁺-induced Ca²⁺ release from neighboring release sites. Instead, experimental evidence and mathematical modeling indicate that this phenomenon is best explained by largely autonomous, local Ca²⁺ release occurring throughout the cytoplasm mediated by both ryanodine receptors (RyR) and InsP₃R [80, 55].

Importantly this secretagogue-induced Ca²⁺ release is modulated in both mouse and human parotid acinar cells under conditions where cAMP is elevated. cAMP results in a substantial increase in the [Ca²⁺]ᵢ signal upon muscarinic stimulation relative to stimulation in the absence of cAMP. In particular the initial increase in [Ca²⁺]ᵢ was enhanced, sub-threshold stimulation was transformed to a measurable [Ca²⁺]ᵢ increase and an oscillatory increase was converted to a sustained [Ca²⁺]ᵢ increase, all consistent with a left shift in sensitivity to stimulation by Ca²⁺ mobilizing agonists. Although, there are obviously many potential molecular targets of cAMP, these studies indicated that modulation by cAMP of PLC activity, pumping activity through PMCA or SERCA, and release through RyR did not appear to be responsible for the enhanced [Ca²⁺]ᵢ signal [29, 57]. Instead the major effect appeared to be enhanced Ca release through PKA phosphorylation of InsP₃R. Moreover, this effect was attributed in particular to the InsP₃R-II since this isoform has the greatest sensitivity to InSP₃, is the most abundant in mouse parotid, and was shown to be specifically phosphorylated [29]. Since InsP₃R are central to mobilizing [Ca²⁺]ᵢ in parotid acinar cells, their regulation by phosphorylation provides a point of convergence whereby any secretagogues acting through Gq-PLC are subject to modulation. Indeed, this appears to be the case since Ca²⁺ release through P2Y purinoreceptors in mouse acini is also is enhanced by cAMP treatment.

While the functional consequences of InsP₃R phosphorylation appear to be sub-type specific, recent studies in heterologous expression systems have demonstrated that phosphorylation of InsP₃R-I in addition to InsP₃R-II also leads to markedly enhanced Ca²⁺ release. The particular sites within InsP₃R-I which are functionally important for potentiated release have also been defined; two canonical PKA-phosphorylation motifs which are conserved from Drosophila to human are present in the InsP₃R-1 sequence but are not present in either the InsP₃R-II or InsP₃R-III [12]. In the splice variant of the InsP₃R-1 expressed in parotid acinar cells (S2⁻ InsP₃R-1 or “short form”) [7] phosphorylation of both sites occurs, resulting in InsP₃R with an enhanced apparent sensitivity to InsP₃. Because potentiation of Ca²⁺ release at the level of InsP₃R can be a nexus for interactions between the cAMP and Ca²⁺ signaling systems in parotid acinar cells, this project investigates the mechanism underpinning this effect by studying InsP₃R with phosphomimetic mutations. These receptors can be used therapeutically to maximize [Ca²⁺]ᵢ signals if expressed in diseased salivary tissue.

### InsP₃-Dependent Ca²⁺ Release: cAMP Effects on Ca⁺ Release

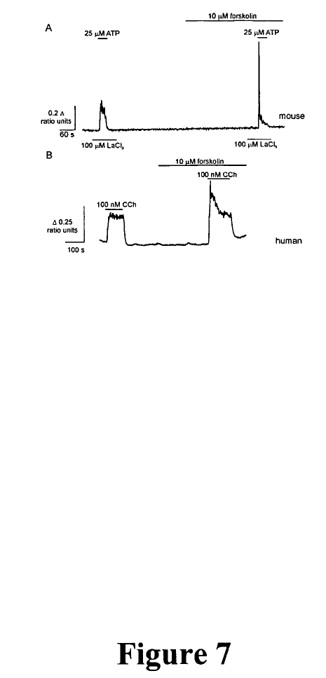

In mouse parotid acinar cells raising cAMP profoundly potentiates muscarinic agonist-induced [Ca²⁺]ᵢ signals [29, 83, 84]. A major mechanism underlying this phenomenon can be the PKA-dependent phosphorylation of InsP₃R [29]. The primary evidence supporting this is that cAMP elevation results in both InsP₃R phosphorylation and a marked potentiation of Ca²⁺ release in a PKA dependent manner [29]. Data, consistent with the idea that InsP₃R are central to this effect, is presented in FIG. 7. First, Ca²⁺ release generated through other Gq/PLC-linked receptors, such as P2Y purinergic receptors were also enhanced by cAMP elevation (FIG. 7A). Secondly, the effect was not species specific since a similar marked potentiation of the CCh-induced elevation in [Ca²⁺]ᵢ was observed in human parotid acini incubated with either forskolin (FIG. 7B) or P-adrenergic agonist prior to stimulation. The effects of InsP₃R phosphorylation can be subtype-specific. Enhanced

Ca²⁺ release is consistent with an effect on InsP₃R-II as this InsP₃R is most abundant in parotid tissue and PKA-dependent phosphorylation of InsP₃R-II has been shown to augment Ca²⁺ release in other tissues such as liver [30]. It should be noted however, that all three subtypes of InsP₃R are expressed in parotid tissue with similar localization to the apical domain of the cell.

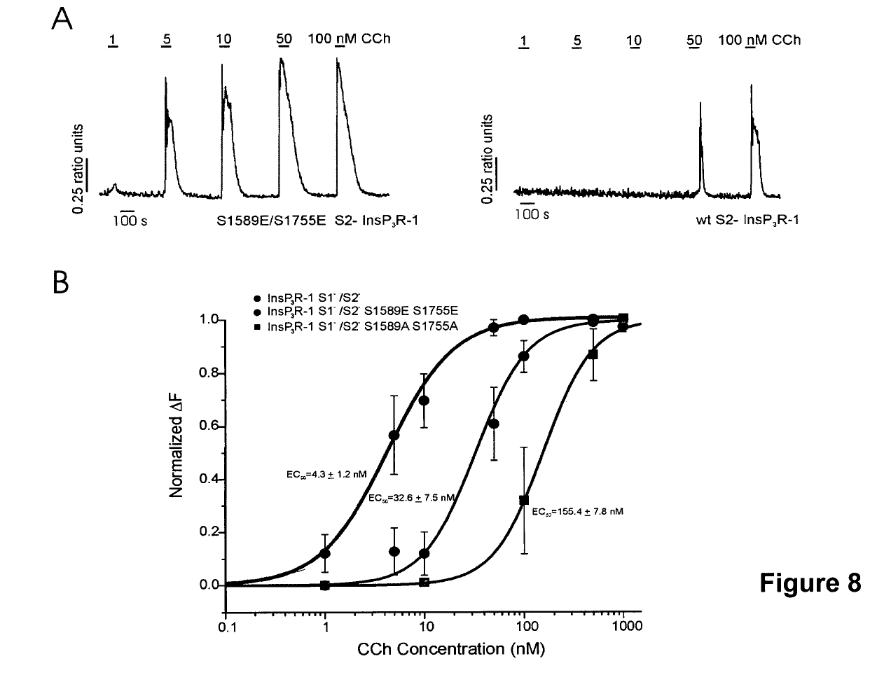

Experiments expressing in the DT-40 3ko cells a mutant InSP₃ R-1 where serine residues at position 1589 and 1755 are mutated to glutamate as described above, result in markedly potentiated Ca²⁺ release (FIG. 8).

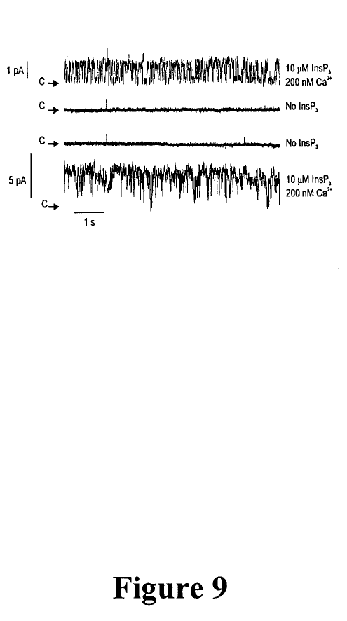

Phosphorylation of InsP₃R can alter apparent sensitivity to InSP₃ by a number of mechanisms. These include altering the sensitivity to InSP₃ itself, by changing the sensitivity of the receptor to its co-agonist Ca²⁺ or alternatively by modulating the interaction with other regulatory factors such as proteins or adenine nucleotides. Although studying Ca²⁺ release in DT-40 3ko cells as described above provides a powerful system to assess the net cellular effect of phosphorylation, a complimentary approach can be employed to study the biophysical properties of single InsP₃R channels in isolated nuclei from cells overexpressing InsP₃R and mutants. This technique relies on the fact that the outer nuclear membrane is continuous with the ER and can be patch clamped in the “on nucleus” configuration [11, 85-89]. Studies have successfully recorded InSP₃ dependent currents from isolated Cos-7 nuclei overexpressing InsP₃R-1 (FIG. 9). Because of the very low expression of endogenous InsP₃R in Cos-7 cells [11, 88] InsP₃-dependent channel activity in untransfected/mock transfected cells has not been observed. In the example shown in FIG. 9, representative sweeps are shown from a single nucleus which was patched on 6 separate occasions; initially with saturating InsP₃, subsequently with no InsP₃, and then later again with InsP₃ in the patch pipette.

### Example 3

### Modulation of Ca²⁺ Release by Inositol 1,4,5-trisphosphate Receptor Phosphorylation

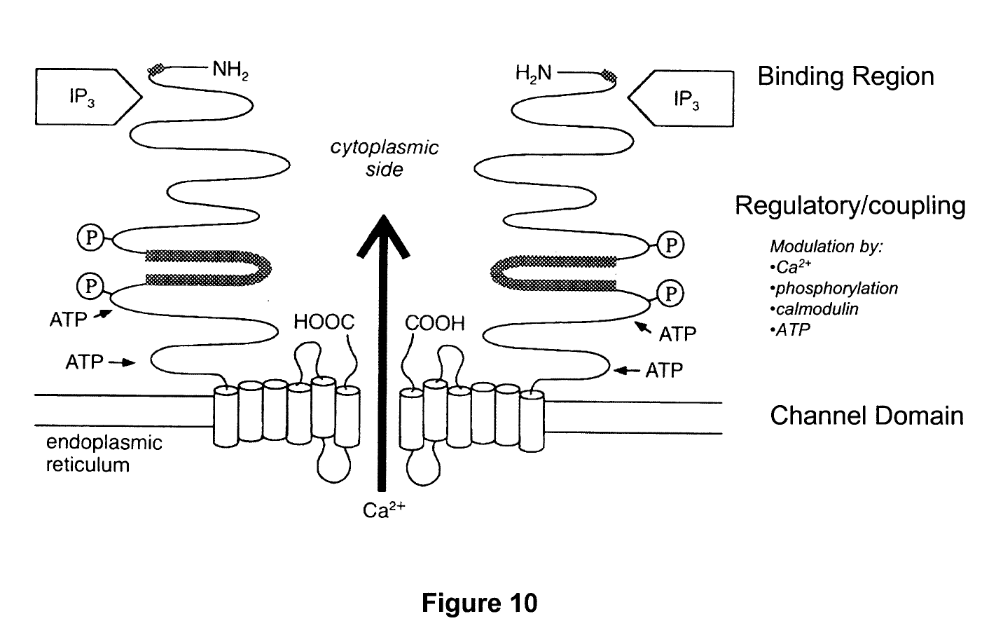

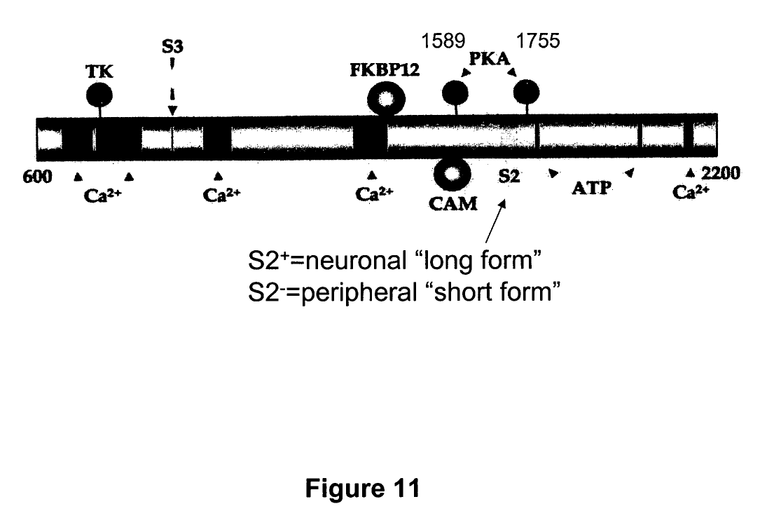

Inositol 1,4,5-trisphosphate receptors (InsP₃R) are the major route of intracellular calcium release in eukaryotic cells and thus are pivotal for stimulation of Ca²⁺ dependent effectors important for the control of numerous physiological processes (FIGS. 10 and 11). Modulation of Ca²⁺ release through InsP₃R is thus of general importance for defining the particular spatio-temporal characteristics of Ca²⁺ signals. While it is widely appreciated that Ca²⁺ itself is an important regulator of InsP₃R, the receptor is also subject to modulation through numerous inputs, including protein-protein interactions, binding of adenine nucleotides and phosphorylation by multiple kinases. In this study, the effects on Ca²⁺ release of phosphorylation of InsP₃R by cyclic nucleotide-dependent protein kinases was studied.

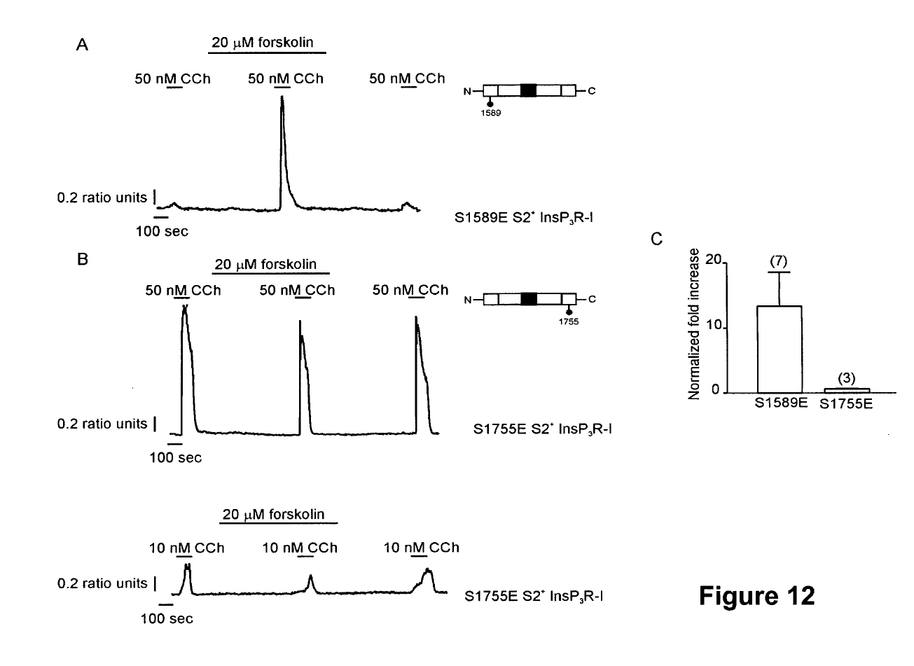

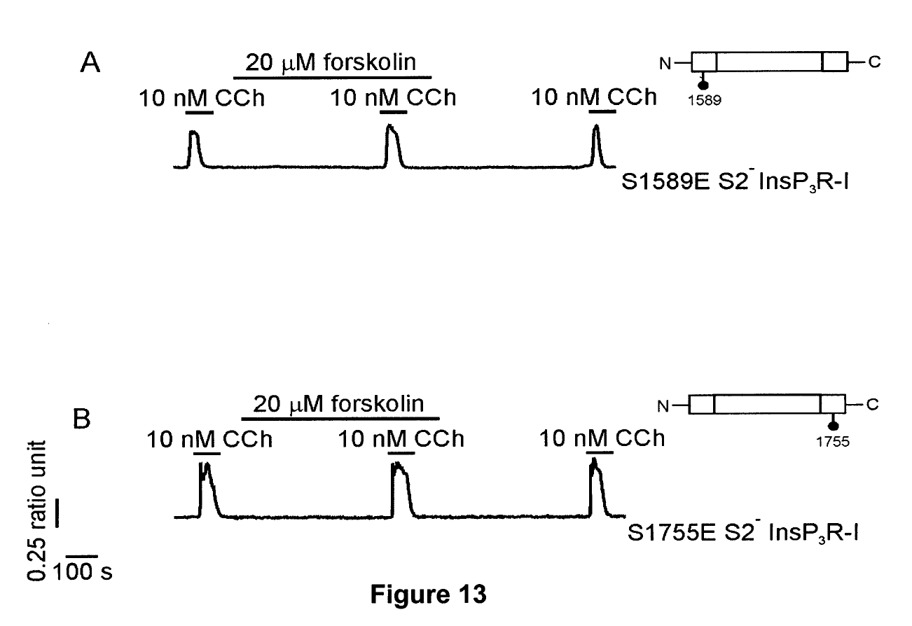

To investigate the particular sites of phosphoregulation in an unambiguously homogenous population of homomeric receptors, type-1 InsP₃R (InsP₃R-I) were expressed in DT-40 cells devoid of endogenous InsP₃R (FIG. 1A) (43). In cells expressing the neuronal, S2⁺ splice variant of the InsP₃R-1, Ca2⁺ release was markedly enhanced when either PKA or PKG was activated (FIG. 1B-D and as described above) (23). The sites of phosphorylation were investigated by mutation of serine residues present in two canonical phosphorylation sites present in the protein. Potentiated Ca²⁺ release was abolished when serine 1755 was mutated to alanine (S1755A) but was unaffected by a similar mutation of serine 1589 (S1589A) (FIGS. 3 and 12). These data demonstrate that S1755 is the functionally important residue for phosphoregulation by PKA and PKG in the neuronal variant of the InsP₃R-1. Activation of PKA also resulted in potentiated Ca²⁺ release in cells expressing the non-neuronal, S2⁻ splice variant of the InsP₃R-1. However, the PKA-induced potentiation was still evident in S1589A or S1755A InsP₃R-1 mutants. The effect was abolished in the double (S1589A/S1755A) mutant, indicating both sites are phosphorylated and contribute to the functional effect (FIG. 8). Indeed, mimicking phosphorylation by changing either S1589 or S1755 to a positively charged glutamate residue (S1589E or S1755E) resulted in InsP₃R with apparent enhanced sensitivity to InSP₃ (FIGS. 8 and 13). Activation of PKG had no effect on Ca²⁺ release in cells expressing the S2⁻ variant of InsP₃R-1 . Collectively these data indicated that phosphoregulation of InsP₃R-1 had dramatic effects on Ca²⁺ release and defined the molecular sites phosphorylated in the major variants expressed in neuronal and peripheral tissues.

### Example 4

### Functional Consequences of Phosphomimetic Mutations at Key cAMP-Dependent Protein Kinase Phosphorylation Sites in the Type I Inositol 1,4,5-Triphosphate Receptor

Two protein kinase A (PKA) consensus sites (RRXS) at Ser-1589 and Ser-1755 are present in the InsP₃R-1 (7, 99), and the most recent studies suggest that phosphorylation of these sites results in a marked enhancement of Ca²⁺ release (23, 24, 26, 100). Most interestingly, these sites are conserved through evolution from Drosophila to humans in InsP₃R-1, but corresponding sites are not present in either the InsP3R-2 or InsP₃R-3. It should be noted, however, that other regions, which are presently not defined, appear to function as PKA phosphorylation sites in InsP₃R-2 and InsP₃R-3. Several reports have demonstrated biochemically that both Ser-1589 and Ser-1755 can be phosphorylated in InsP₃R-1 (34, 35, 99); however, these studies have not provided a consensus as to which sites are important physiologically in tissue that expresses either of the two major splice variants of InsP₃R-1.

These studies were performed by using mutational analysis by substituting alanine for the serine residues present at individual putative phosphorylation sites. Although phosphorylation of both neuronal (S2+) and peripheral (S2−) forms of InsP₃R-1 by PKA resulted in enhanced Ca²⁺ release, mutational analysis indicated that only phosphorylation of Ser-1755 was functionally important in the neuronal S2⁺ InsP3R-1. In contrast, both Ser-1589 and Ser-1755 appeared to be phosphorylated and significant in the peripheral S2⁻ form of InsP₃R-1. In addition, although the S2+ form of the receptor was subject to direct phosphoregulation by cGMP-dependent protein kinase (PKG), the S2− form was not influenced by activation of PKG. These data represent one of the few major differences reported for the regulation of the two major splice variants of the InsP₃R-1.

Herein, charge mutations were constructed, substituting glutamate residues for the serine residues in the functionally important phosphorylation sites in both S2+ and S2− variants of the InsP₃R-1. These mutations can mimic phosphorylation and allow the assessment of the functional effects of phosphorylation of InsP₃R-1. Most importantly, the Ca²⁺ release properties of phosphomimetic mutations can be essentially independent of cell typespecific factors, including the expression of accessory proteins such as protein A-kinase anchoring proteins. These effects can be unambiguously specific to InsP₃R and thus independent of confounding PKA effects on other Ca²⁺-handling machinery. This latter consideration has historically plagued the functional assessment of PKA phosphorylation of InsP₃R. These mutations have allowed us to define the relative sensitivity of Ca²⁺ release of the phosphomimetic mutations and to confirm which sites are important in each splice variant.

The present study has also addressed whether phosphorylation of individual sites is permissive or additive in each splice variant, and the consequences of phosphorylating InsP₃R-1 on Ca²⁺ oscillations, the physiological pattern of Ca²⁺ signaling in nonelectrically excitable cells was investigated.

### Materials and Methods

The acetoxymethyl esters of Fura-2 and Fluo-4 were purchased from Molecular Probes (Eugene, Oreg.). Cell-permeable cyclic nucleotides and forskolin were purchased from Biomol (Plymouth Meeting, Pa.). All other chemicals were purchased from Sigma. The Dt-40 cells lacking InsP3R (Dt-40 3ko) were kindly provided by Dr. Kurosaki (Kansai Medical University, Japan) and were maintained as described previously (42-44).

### Production of Mutations

The rat S2⁺ InsP₃R-1 in the expression plasmid pIRES-GFP was digested with the restriction endonuclease SalI. The overhang created by digestion was blunted by using T4 polymerase. An EcoRI linker was then ligated onto the blunted ends of the construct. The entire receptor DNA was excised from the plasmid using EcoRI and ligated into the plasmid MXT-1. The region containing the S2⁻ splice variant and potential PKA phosphorylation sites was excised from its backbone in pCDNA 3.1+ by RsrII and KasI and ligated into the InsP3R construct in MXT-1. The potential PKA phosphorylation sites Ser-1589 and Ser-1755 were mutated, individually in both splice variants and together in the S2⁻ splice variant, to alanines or glutamates by using sequential PCR mutagenesis. The outside primers used for the mutagenesis reaction flanked the restriction sites RsrII and KasI. Following mutation, the resulting fragments were cut with RsrII and KasI and inserted into the InsP₃R-1 backbone at the corresponding sites. The mutations were confirmed by Big Dye fluorescent sequencing. Mutated receptor DNAs were excised from MXT-1 by using EcoRI and ligated into the mammalian expression vector pGW (provided by Dr. David Yue, Johns Hopkins University). Orientation was confirmed by using restriction enzyme digestion. Mutants were named based on the splice variant, either S2+ or S2− followed by the amino acid present at position 1589 and 1755. Thus, a mutation in S2^{+ InsP}₃R-1 S1755E is designated, “S2+SE,” and in InsP₃R-1 S2+ S1755A is designated “S2⁻SA”. Similarly, an S2− InsP₃R-1 with mutations in both S1589E and S1755E is designated “S2−EE”. The numbering of residues is based on the full-length rat InsP₃R-1.

### Transfection of Dt-40 Cells

Dt-40 cells lacking all three InSP₃ receptor subtypes were transfected by using electroporation at 350 V and 950 microfarads (4-mm gap cuvette). 2×10⁷ cells were co-transfected with 25 μg of the InsP₃R-1 cDNA, 25 μof the muscarinic type 3 (m3R) receptor DNA, and 4 μg of the red fluorescent protein plasmid pHcRed1-N1 (Clontech). Cells were incubated with DNA in 500 μl of Opti-MEM media (Invitrogen) on ice for 10 min. The cell/DNA mixture was electroporated, incubated on ice for 30 min, increased to 5 ml with Opti-MEM, and placed in a 5% CO₂ incubator at 39° C. for 5 h. The cells were then centrifuged and resuspended in 12 ml of complete RPMI media (Invitrogen). Transfection efficiency was typically ˜20%. Experiments were performed within 32 h of transfection.

### Transfection of HEK-293 Cells and Assessment of Phosphorylation of S2− InsP₃R-1

HEK-293 cells were plated onto 25-cm² culture flasks and allowed to grow to near-confluency. Cells were transfected with 5 μg of each S2− InsP₃R-1 DNA construct by using the LipofectAMINE reagent (Invitrogen) as per the manufacturer's instructions. The following day, batches of cells were treated in the presence or absence of 20 μM forskolin for 10 min, aspirated from flasks, lysed, and immunoprecipitated with a polyclonal α-InsP₃R-1 antibody that recognizes amino acids 2731-2749 of InsP₃R-1. Immunoprecipitates were separated on 5% SDS gels transferred to nitrocellulose and then probed with either the α-InsP₃R-1 antibody or a polyclonal antibody that recognizes the phosphorylated state of Ser-1755 (39) (α-phospho-Ser-1755), kindly provided by Dr. S. Snyder. Blots that were probed with a-phospho-Ser-1755were stripped and reprobed with the α-InsP₃R-1 antisera to confirm the presence and relative quantity of the InsP₃R-1.

### Digital Imaging of [Ca²⁺]ᵢ

Transfected Dt-40 3ko cells were washed once in a HEPES-buffered physiological saline solution (HEPES-PSS) containing (in mM) 5.5 glucose, 137 NaCl, 0.56 MgCl2, 4.7 KCl, 1 Na2HPO4, 10 HEPES (pH 7.4), 1.2 CaCl2, and 1% w/v bovine serum albumin. Cells were then resuspended in bovine serum albumin HEPES-PSS with 1 μM Fura-2 (AM), placed on a 15-mm glass coverslip in a low volume perfusion chamber, and allowed to adhere for 30 min at room temperature. Cells were perfused continuously for 10 min with HEPES-PSS before experimentation to allow complete Fura-2 de-esterification. A field of cells for each experiment was chosen that contained a wide range of transfection efficiency based upon the intensity of red fluorescence emitted when excited at 560 nm. Individual cells that had emission gray levels between 1500 and 2500 were subsequently chosen to standardize expression levels. [Ca²⁺]ᵢ imaging was performed essentially as described previously by using an inverted epifluorescence Nikon microscope with a 40× oil immersion objective lens (numerical aperture, 1.3) (100). Cells were excited alternately with light at 340 and 380±10 nm bandpass filters (Chroma, Rockingham, Vt.) using a monochrometer (TILL Photonics, Pleasanton, Calif.). Fluorescence images were captured and digitized with a digital camera driven by TILL Photonics software. Images were captured every 2 s with an exposure of 2 ms and 4 by 4 binning. 340/380 ratio images were calculated online and stored immediately to a hard disk.

### Flash Photolysis

Transfected cells were simultaneously loaded with the visible wavelength indicator Fluo-4 and a cell-permeable form of caged inositol trisphosphate (ci-IP₃/PM) for 30 min. ci-IP₃/PM is a homologue of cm-IP₃/PM. The 2- and 3-hydroxyls of the inositol ring are protected by an isopropylidene group in ci-IP₃/PM and are protected by a methoxymethylene group in cm-IP₃/PM (45). Like cm-IP₃/PM, ci- IP₃/PM diffuses across cell membranes, and the PM group is hydrolyzed by cellular esterases, and Ca²⁺ release can be induced upon photouncaging as i-IP₃ is liberated from the cage and acts in a similar fashion to InSP₃ at InsP₃R². A further period of 30 min was allowed for de-esterification of both dye and cage. Cells were illuminated at 488±10 nm and fluorescence collected through a 525±25-nm bandpass filter and captured using the Till Photonics imaging suite. These traces are displayed as % ΔF/Fₒ, where F is the recorded fluorescence, and Fₒ is the mean of the initial 10 sequential frames. Photolytic release was performed as described previously by using a pulsed xenon arc lamp (Till Photonics). A high intensity (0.5-5 ms duration; 80 J) discharge of UV light (360±7.5 nm) was reflected onto the plane of focus by using a DM400 dichroic mirror and Nikon 40× oil immersion objective, 1.3 NA.

### Frequency Distribution of Ca²⁺ Oscillations

Cells transfected with WT and mutant S2− isoforms were stimulated with varying concentrations of an α-IgM antibody (Southern Biotechnology Associates, Inc., Birmingham, Ala.). Infrequent Ca²⁺ oscillations were produced presumably through B cell receptor cross-linking, activation of phospholipase C-γ, and subsequent production of InsP₃. The frequency of Ca²⁺ oscillations was determined by selecting individual peaks that displayed an increase in ratio units greater than 0.05 and are listed as frequency in milliHertz (number of oscillations in 1000 s).

### Concentration-Response Relationships

Normalized ΔF concentration-response relationships were fit with the following logistic.

Equation 1,

ΔF=1/(1+(C/EC₅₀)^{SlopeΔF} (Eq. 1)

where ΔF is the change in fluorescence normalized to the maximal response; C is agent concentration; EC₅₀ is the concentration where the response is half of maximum, and SlopeΔF is a slope factor related to the Hill coefficient.

### Statistical Analysis

The effects of treatment were determined by normalizing the peak change in fluorescence ratio by stimulation following forskolin or 8-Br-cGMP exposure to that of stimulation in control HEPES-PSS. Thus, pooled data represent a normalized fold increase over control for the treated trial. In all cases where statistical significance is indicated, two-tailed heteroscedastic t tests were performed. P values<0.05 were considered to indicate statistical significance and are denoted by an asterisk in the figures.

### Phosphomimetic Mutations in Functionally Important Phosphorylation Sites in Both S2+ and S2− InsP₃R-1 Result in Enhanced Ca²⁺ Release

Phosphorylation of proteins results in the addition of net negative charge to the phosphoacceptor residue. In the case of PKA phosphoregulation, the functional effects of phosphorylation are thought to occur as the negative charge added to the serine or (less frequently) threonine residues neutralizes the positive charge of basic arginine or lysine residues present upstream in the classical RRX(S/T) consensus motif (101). This charge neutralization, in turn, is thought to result in a conformational change in the protein. A common approach employed to investigate the functional effects of phosphorylation is to construct phosphomimetic mutations whereby glutamic or aspartic acid residues are substituted at the phosphoacceptor site (102, 103). The rationale for this strategy is that the negatively charged side chain of the substituted acidic amino acid will mimic, to an extent, the addition of a phosphate moiety to the protein.

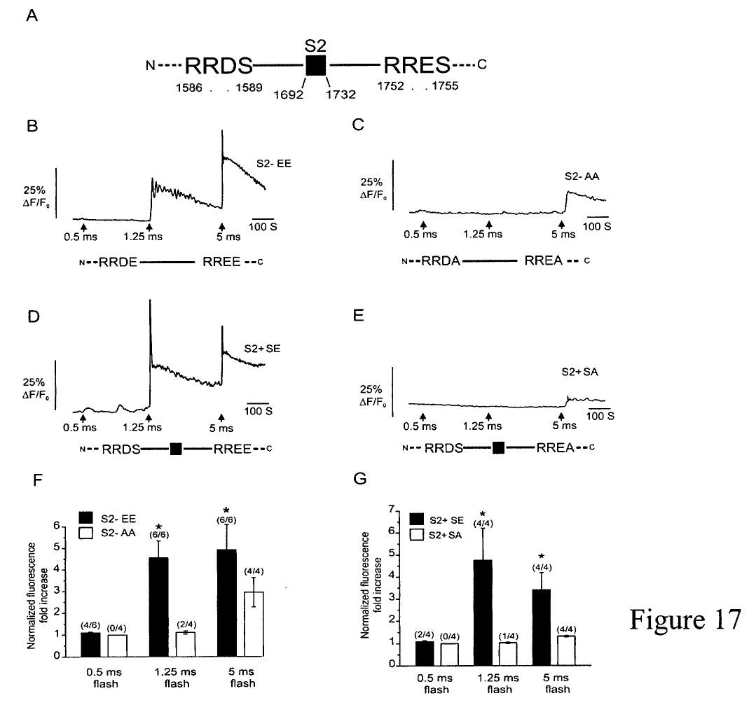

To investigate the consequences of InsP₃R-1 phosphorylation, the Ca²⁺ release properties of phosphomimetic mutations in the functionally important sites in both S2+ and S2− InsP₃R-1 was analyzed in Dt-40 3ko cells. This cell line provides the only known InsP₃R null background (42, 43). In initial experiments, a comparison was made between the sensitivity of Ca²⁺ release by InsP₃R-1 phosphomimetic mutations versus nonphosphorylatable alanine mutations at the sites. Dt-40 3ko cells were transfected with DNA encoding HcRed to facilitate identification of transfected cells and either S2− S1589E/S1755E InsP₃R-1 (S2−EE) or S2− S1589A/S1755A (S2−AA). Ca²⁺ release was monitored following flash photolysis of ci-IP₃, a cell-permeable form of caged InSP₃. This experimental paradigm provides a relatively direct assessment of the effects of InsP₃R-1 phosphorylation on the Ca²⁺ release process. The amount of ci-IP₃ photo-released was controlled by varying the duration of the UV flash discharge (0.5-5 ms). No increase in [Ca²⁺]ᵢ was observed following the longest UV discharge in cells either not loaded with ci-IP₃/PM or not expressing HcRed. FIGS. 17, B and C, shows traces from typical experiments in individual Dt-40 3ko cells expressing S2−EE or S2−AA (FIGS. 17, B and C, respectively, and pooled data in FIG. 17F). In cells expressing S2−EE, Ca²⁺ release as defined by a >0.05% ΔF/F, increase in initial fluo-4 fluorescence was observed in ˜60% of cells when exposed to UV discharge for 0.5 ms. Subsequent exposure to UV light for 1.25 ms elicited a more robust increase in [Ca²⁺]ᵢ in all cells. Finally, photo-release following a 5-ms flash, in general evoked a further increase in the magnitude of Ca²⁺ release. In contrast, an elevation of intracellular Ca²⁺ was never observed under identical conditions following a 0.5-ms UV flash in cells expressing S2−AA. A significant Ca²⁺ release was only observed in ˜50% of cells following a 1.25-ms flash, and the majority of cells only responded to the longest uncaging duration, albeit with a smaller magnitude than in S2−EE-expressing cells exposed to the same stimulus.

A similar pattern of sensitivity was observed in Dt-40 3ko cells expressing S2+ S1755E (S2+SE) and S2+ S 1755A (S2+SA) as shown in FIGS. 17, D and E, respectively, and pooled data in FIG. 17G. Cells expressing S2+SE responded more robustly to photolysis of ci-IP3 than cells expressing S2+SA exposed to an identical stimulus. These data indicate that serine to glutamate mutations at the functionally important phosphorylation sites in both splice variants of InsP₃R-1 are “phosphomimetic,” i.e. charge mutations mimic phosphorylation in that these constructs display an apparent increased functional sensitivity to InsP₃.

### Sensitivity of S2−InsP₃R-1 and Phosphorylation Site Mutants

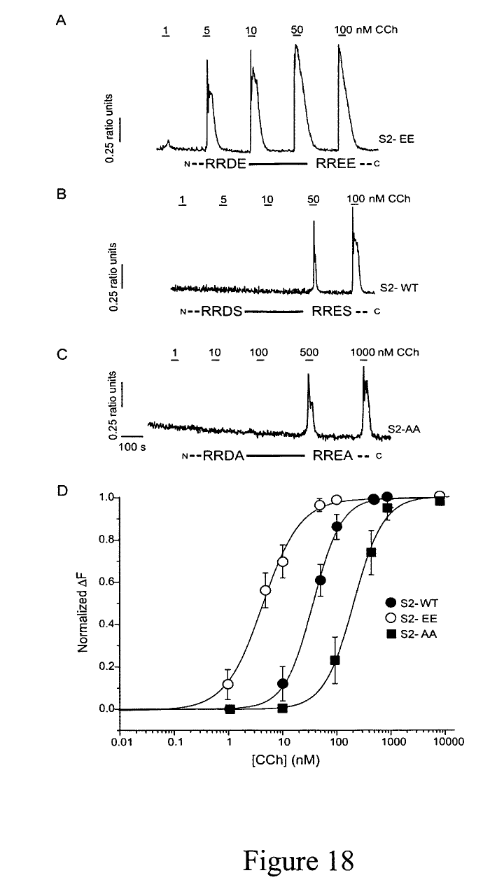

Experiments were next performed to determine the relative sensitivity of Ca²⁺ release through the phosphomimetic mutations of the S2− InsP₃R-1 with respect to the wild type and phosphorylation-deficient mutants. Dt-40 3ko cells were transfected with cDNAs encoding m3R, HcRed together with the InsP₃R-1 construct of interest. Stimulation with the muscarinic agonist CCh results in robust increases in [Ca²⁺]ᵢ in transfected cells through the Gαq/11-coupled stimulation of phospholipase C-β and subsequent formation of InsP3. The magnitude of the initial peak provides a good estimation of the extent of Ca²⁺ release as this parameter in Dt-40 cells, like many cells, is essentially independent of Ca²⁺ influx. Furthermore, in Dt-40 cells the [Ca²⁺]ᵢ response to stimulation, with CCh does not appreciably desensitize, and thus the effects of multiple concentrations of agonist can be assessed in a single cell. Individual HcRed-expressing cells were stimulated with increasing concentrations of CCh (1 nM to 1 μM) for 60 s followed by a 5-min wash between applications of agonist. In each case, these experiments were performed on multiple cells, expressing a narrow range or HcRed fluorescence and from multiple batches of transfected cells to minimize variation because of expression level of m3R and InsP₃R-1. Concentration-response relationships were generated by normalizing each initial peak to the maximum response in the individual cell and subsequently averaging the pool of cells expressing a particular construct. FIG. 18A shows a typical example of this experimental procedure performed on Dt-40 3ko cells expressing S2−EE. A significant increase in Ca²⁺ was detected in the majority of S2−EE-expressing cells following stimulation with 1 nM CCh, and the peak response occurred following stimulation between 10 and 50 nM CCh. In contrast, as shown in FIGS. 18, B and C, Ca²⁺ release following CCh stimulation in the wild type S2−InsP₃R-1 or S2−AA was considerably less sensitive to CCh stimulation. Analysis of the pooled data indicated that the magnitude of the peak response to any of these constructs was not significantly different (maximum peak Δ response: S2−WT=0.9±0.1 ratio units; S2⁻ AA=0.67+0.1 ratio units, and S2−EE=1.13+0.1 ratio units), indicating that the efficacy of Ca²⁺ release was essentially unaltered. However, when an estimate of the relative sensitivity was made by fitting the normalized concentration-response relationships for each construct (FIG. 18D), CCh-induced Ca²⁺ release in the S2− mutant (EC₅₀ 4.3±1.2 nM CCh) was 7.5-fold more sensitive when compared with WT S2−InsP₃R-1 (EC₅₀=32.6±7.5 nM) and some 50-fold more sensitive than the nonphosphorylatable S2−AA mutant (EC₅₀ =229.5±14.6 nM). These data provide strong evidence that the S2−EE construct is more sensitive to stimulation by InsP₃ and present evidence that phosphorylation of the InsP₃R-1 results in marked regulation of channel function. This profound regulation could be expected to have major consequences for calcium signaling events in peripheral tissue such as liver, testis, and smooth muscle which express the S2-InsP₃R-1 (7, 90). The observation that S2⁻ AA is relatively less sensitive than S2−WT is consistent with the possibility that a proportion of the wild type receptor is constitutively phosphorylated in Dt-40 cells, thus contributing to the intermediate sensitivity of the wild type S2−InsP₃R-1 relative to the phosphomimetic S2−InsP₃R-1 receptor.

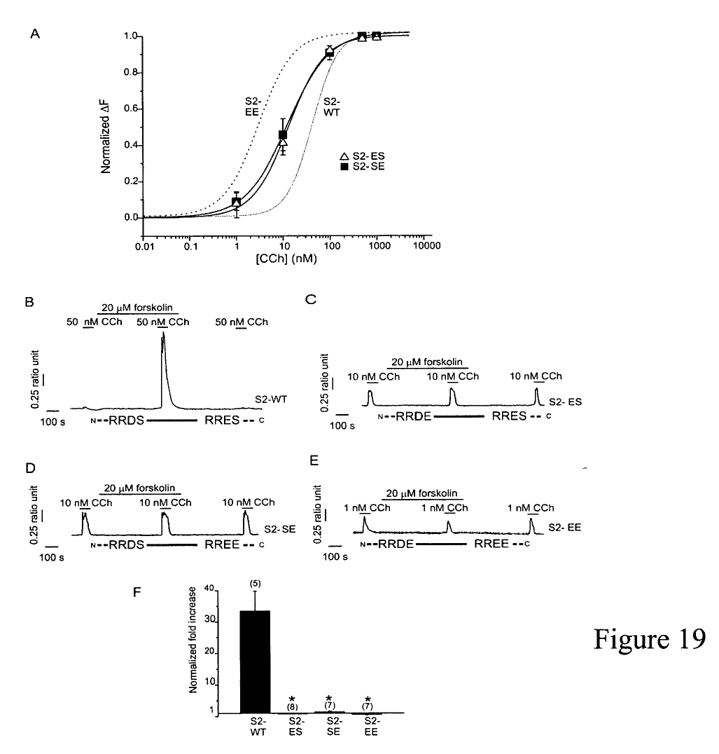

Although it has been demonstrated that each site can be phosphorylated, it is not definitively known whether the functional effects of phosphorylating individual sites are independent and additive or alternatively if the full effect is seen following phosphorylation of an individual site. Thus, experiments were next performed to assess the sensitivity of single phosphomimetic mutations within each phosphorylation site. Concentration-response relationships for CCh-induced Ca²⁺ release were constructed for Dt-40 3ko cells expressing either S2−S1755E InsP₃R-1 (S2−SE) or S2−S1589E InsP₃R-1 (S2−ES). The data were analyzed as described previously for FIG. 18D. FIG. 19A shows the fit for the normalized concentration-response relationship for S2−SE and S2−ES and for comparison also shows the fit for S2−EE and S2−WT (FIG. 19A, dotted lines; data from FIG. 18D). Once again the maximal initial peak responses in either mutant were not significantly altered from wild type (S2−ES=0.54±0.1 Δ ratio units; S2−SE=0.78±0.1 Δ ratio units); however, the sensitivity of each of these mutants was significantly shifted, such that the EC₅₀ for CCh-induced Ca²⁺ release was enhanced ˜3-fold over the response in wild type for either mutant (EC₅₀ for CCh-induced release: S2−ES=12.4±0.5 nM; S2−SE=13.5±1 nM). These data are summarized in Table 2.

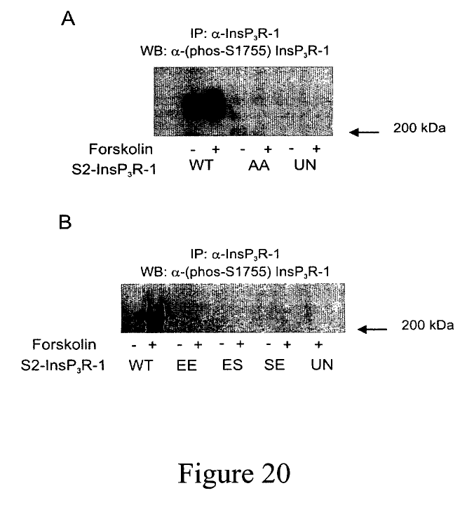

The enhanced apparent sensitivity of individual phosphomimetic mutants was essentially equal and intermediate between the sensitivity of the S2−WT and S2−EE mutations. These data are consistent with phosphorylation of individual sites being functionally additive. To address this possibility directly, experiments were performed evaluating the effects of activating endogenous PKA in cells expressing S2−SE or S2−ES to mimic prior phosphorylation of an individual site. As shown in FIG. 19B, and reported previously, activation of PKA by incubation with forskolin results in a dramatic potentiation of Ca²⁺ release in Dt-40 3ko cells expressing S2−WT (100). Most surprisingly, although the sensitivity to CCh was enhanced, as evidenced by the low concentration of CCh necessary to evoke threshold Ca²⁺ release, no potentiation of Ca²⁺ release following forskolin incubation was observed in Dt-40 3ko cells expressing either S2−ES (FIG. 19C; pooled data in FIG. 19F), S2⁻SE (FIG. 19D; pooled data in FIG. 19F), or S2−EE (FIG. 19E; pooled data in FIG. 19F). Thus, although each phosphorylation site in S2− InsP₃R-1 can be phosphorylated and mimicking phosphorylation of both sites leads to a receptor with enhanced sensitivity relative to phosphorylation of an individual site, phosphorylation of both sites in situ does not appear to occur and therefore is not functionally additive. Although these data may seem paradoxical, an explanation, consistent with all the observations is that the initial phosphorylation of either Ser-1589 or Ser-1755 leads to a conformational change in the receptor that now precludes the phosphorylation of the additional site. To test this, experiments were performed to determine the phosphorylation state of the various mutants after raising cAMP levels. These experiments were performed in HEK-293 cells because of the high transfection efficiency and low endogenous levels of InsP₃R-1 (47, 99). HEK-293 cells were transfected with the constructs as indicated in FIG. 20, incubated in the presence or absence of forskolin for 10 min, then pelletted, and lysed. Following incubation of the lysates with α-InsP₃R-1 antibody, the immune complexes were captured and separated on SDS gels, and the phosphorylation status of Ser-1755 was determined by Western blotting with an antibody that specifically recognizes this phosphorylated residue in InsP3R-1 (39). As shown in FIG. 20A, wild type S2⁻InsP3R-1was robustly phosphorylated after forskolin incubation, whereas no phosphorylation was detected in wild type S2−AA or untransfected HEK-293 cells. Similarly, in FIG. 20B, a marked increase in phosphorylation could be detected in S2−InsP₃R-1, whereas no phosphorylation could be detected in cells transfected with S2−EE, S2−SE or, most importantly, the S2−ES construct. These data indicate that the initial phosphorylation of one site precludes further phosphorylation at the additional residue as indicated by the functional data and indicate that physiologically only phosphorylation of a single residue is functionally relevant in the S2⁻ splice variant of InsP₃R-1.

### Sensitivity of S2+InsP3R-1 and Phosphorylation Site Mutants

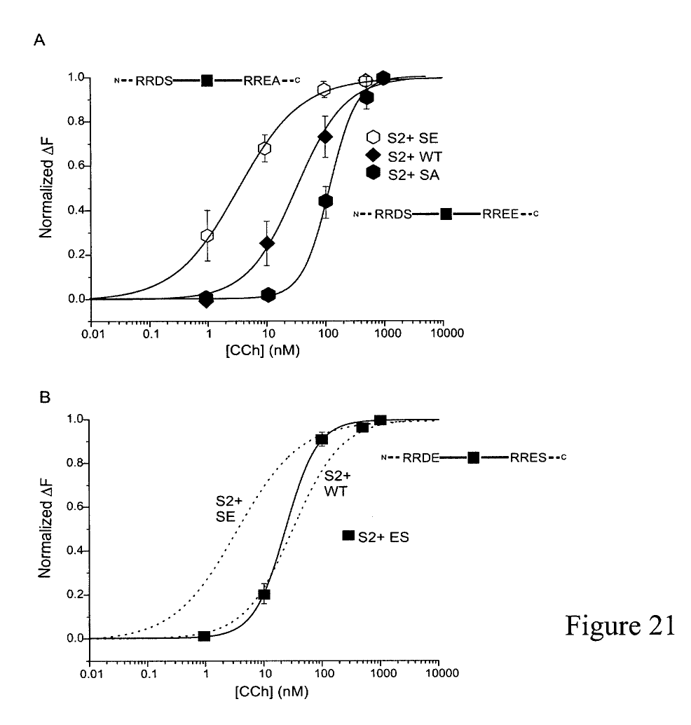

The sensitivity of Ca²⁺ release via the neuronal S2+InsP₃R-1 was next assessed by using a similar experimental paradigm to that used for the S2− form of the receptor. Although biochemically both Ser-1589 and Ser-1755 are equally susceptible to phosphorylation by PKA in S2+InsP₃R-1 (99), only Ser-1755 appears to be functionally relevant in terms of modulating Ca²⁺ release (100). FIG. 21A shows the fits for the normalized concentration-response relationships for CCh-induced Ca²⁺ release in cells expressing S2+WT, phosphomimetic S2+SE, and S2+SA. The apparent sensitivity of CCh induced Ca²⁺ release in cells expressing S2+WT was essentially identical to S2−WT InsP3R-1 (EC₅₀ for CCh-induced Ca²⁺ release; S2⁺InsP₃R-1=32.1±4.2 nM versus S2−32.6±7.5 nM). These data indicate that InsP₃ binding and InsP₃-induced calcium release is identical in the two major splice variants of the InsP₃R-1 (104-106) and is therefore supportive of the contention that the initial CCh-stimulated [Ca²⁺]ᵢ peak is a good indicator of InsP₃R function. In a similar fashion to the S2−InsP3R-1, mutation of Ser-1755 to either alanine or glutamic acid did not significantly alter the maximal initial peak upon CCh stimulation (maximum peak Δ response: S2+WT=0.61±0.1 Δ ratio units; S2+SE=0.59±0.1 Δ ratio units; S2+SA=0.84±0.2 Δ ratio units) but did, however, significantly affect the apparent sensitivity of Ca²⁺ release as shown in FIG. 21A. The S2+SE mutant exhibited a similar EC₅₀ for CCh induced Ca²⁺ release as the S2−EE mutation (EC₅₀: S2+SE=3.6±0.2 nM CCh; S2−EE=4.3 nM), being ˜9-fold more sensitive than S2+WT and ˜32-fold more sensitive than the nonphosphorylatable S2+SA mutation (EC₅o S2+SA=116.3±3 nM CCh). These data are summarized in Table 2.

TABLE 2
Summary of the effects of phosphorylation by PKA or PKG of the S2−/S2+ splice variants of InsP₃R-1 and
phosphoregulatory mutations at Ser-1589 and Ser-1755 expressed in Dt40 3 knock out cells
The data are derived from this paper and Ref. 100. NT, not tested.

| | S2 + InsP₃R-1 | | S2 − InsP₃R-1 | |
|---|---|---|---|---|
| Amino acid at | CCh EC₅₀ (M)/(fold | Ca²⁺ release after PKA/PKG | CCh EC₅₀ (M)/(fold | Ca²⁺ release after PKA/PKG |
| 1589/1755 | change from WT) | activation | change from WT) | activation |
| Wild-type SS | 32.1 ± 4.2/(x1) | Enhanced/enhanced | 32.6 ± 7.5/(x1) | Enhanced/no change |
| AS | NT | Enhanced/enhanced | NT | Enhanced/no change |
| SA | 116.3 ± 3.0/(x0.3) | No change/no change | NT | Enhanced/no change |
| AA | NT | NT/NT | 229.5 ± 14.6/(x0.14) | No change/no change |
| SE | 3.6 ± 0.2/(x8.9) | No change/no change | 13.5 ± 1.1/(x2.4) | No change/NT |
| ES | 23.7 ± 1.2/(x1.3) | Enhanced/enhanced | 12.4 ± 0.5/(x2.6) | No change/NT |
| EE | NT | NT/NT | 4.3 ± 1.2/(x7.6) | No change/NT |

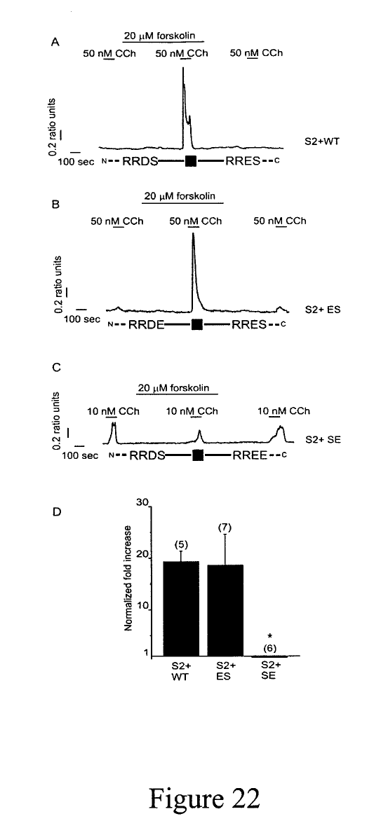

In contrast to the S2+InsP₃R-1 S1755E construct, mutation of Ser-1589 to glutamate in the S2+ form of InsP₃R-1 did not significantly affect the apparent sensitivity of Ca²⁺ release as shown in FIG. 21B. Both the maximum peak response to CCh and the sensitivity of the receptor were very similar to that of S2+WT InsP₃R-1 (maximum peak Δ response=0.59±0.2 Δ ratio units; EC₅₀ =23.7±1.2 nM CCh). However, it is formally possible that phosphorylation of Ser-1589 is only functionally important following phosphorylation of Ser-1755 in S2^{+ InsP}₃R-1. To test this, experiments were performed activating PKA with forskolin in Dt-40 3ko cells expressing S2+ES or SE mutants. Treatment with forskolin resulted in a marked potentiation of CCh-induced Ca²⁺ release in cells expressing either S2+WT or S2+ES (FIGS. 22, A and B, respectively, and pooled data in D) presumably as Ser-1755 was phosphorylated. Although cells expressing S2+SE were more sensitive to CCh, no further potentiation was observed following forskolin incubation (FIG. 22C and pooled data in FIG. 22D). These data indicate that phosphorylation of Ser-1755 is permissive for any functional effect of phosphorylating Ser-1589 in S2+InsP₃R-1.

### Effect of PKG Activation on S2+InsP₃R-1 and Phosphomimetic Mutants

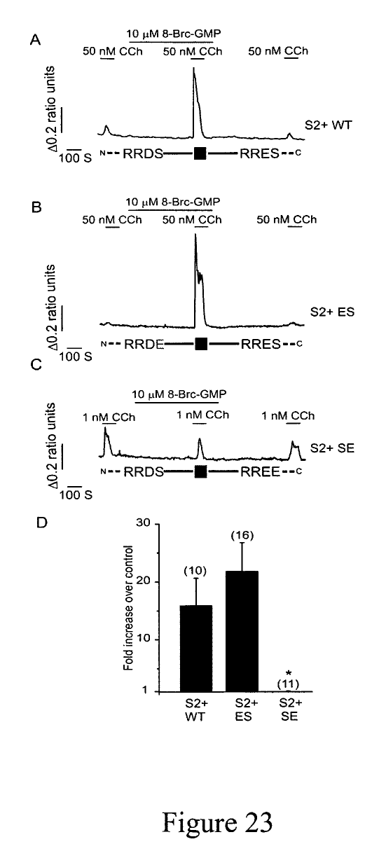

Despite these differences, experiments were performed to define further the phosphorylation sites important for regulation of Ca²⁺ release by PKG. FIG. 23A shows a representative trace from cells expressing S2+WT InsP₃R-1 thus illustrating the marked potentiation of Ca²⁺ release upon specific activation of PKG by 8-BrcGMP. A similar striking potentiation of Ca²⁺ release was also observed in cells expressing S2+ES (FIG. 23B and pooled data FIG. 23D), again presumably as Ser-1755 is phosphorylated following incubation with 8-Br-cGMP. In contrast, in cells expressing S2+SE no effect on CCh-induced Ca²⁺ release following activation of PKG was observed (FIG. 23C and pooled data in FIG. 23D). These data provide evidence that phosphorylation of Ser-1589 by PKG either in isolation or following phosphorylation of Ser-1755 plays a role in modulation Ca²⁺ release through S2+InsP₃R-1.

The specific pattern of phosphorylation occurring upon stimulation of PKA or PKG can be cell type-specific as, for example, in the case of PKA as a result of the targeting through protein A-kinase anchoring proteins, as has been demonstrated recently (107) for InsP₃R. Alternatively, cell-specific effects could conceivably occur through restricted access of the kinase to its substrate. Notwithstanding the general importance of kinase targeting for efficient, localized phosphorylation, the phosphomimetic constructs used in this study reveal the intrinsic, functionally important sites in a manner independent of the particular cellular context because any targeting step is circumvented. These sites and the functional consequences of phosphoregulation can be a general property of the InsP₃R-1. In addition, the particular sites in S2+InSP3R-1 are entirely consistent with the earlier study (100) of the sites functionally important in enhanced Ca²⁺ release following PKA or PKG phosphoregulation, and this reinforces the view that phosphorylation of S2+InsP3R-1 Ser-1589 has no significant role, at least in terms of Ca²⁺ release.

### Effect of InsP3R Phosphorylation on Ca²⁺ Signaling Events

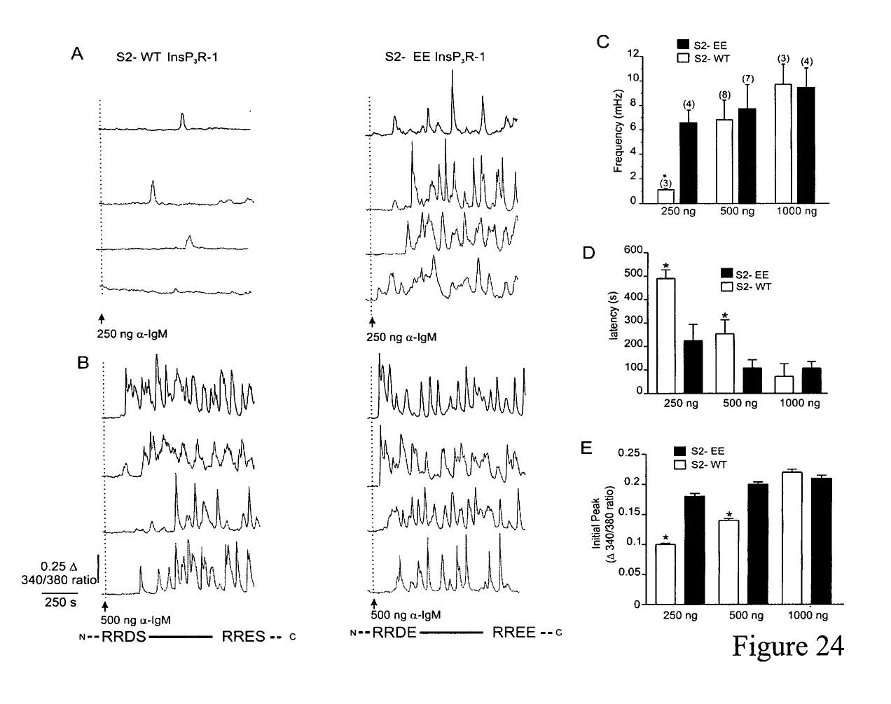

In many nonelectrically excitable cell types, the physiological mode of Ca²⁺ signaling is through the generation of Ca²⁺ oscillations (1, 108). Moreover, it is a generally held view that the spatial and temporal properties of Ca²⁺ oscillations make an important contribution to defining the fidelity and specificity of Ca²⁺ signaling. In many current models addressing the mechanism underlying Ca²⁺ oscillations, a key feature is the regulation of Ca²⁺ release through InsP₃R. Experiments were then designed to assess the consequences of InsP₃R-1 phosphorylation (expressed in isolation) on the initiation and generation of Ca²⁺ oscillations. An α-IgM antibody was chosen to stimulate and to initiate Ca²⁺ oscillations through activation of the endogenous B cell receptor, phospholipase C-γ activation, and the formation of InsP₃. Dt-40 3ko cells expressing either S2−WT or S2−EE to mimic PKA phosphorylation of the InsP₃R-1 were stimulated with various concentrations of α-IgM antibody as illustrated by the selection of representative traces in FIG. 24. Stimulation with 250 ng/ml α-IgM proved to be a threshold concentration in S2−WT-expressing cells. This degree of stimulation generally resulted in a single small increase in [Ca²⁺]ᵢ after a long latency (FIG. 24A, left panel, and pooled data in FIG. 24, C-E). An identical stimulus in cells expressing S2−EE, in contrast, resulted in repetitive Ca²⁺ transients following a much shorter latency, consistent with the increased apparent sensitivity of the S2−EE constructs (FIG. 24A, right panel, and pooled data FIG. 24, C-E). Most interestingly, stimulation of S2−WT-expressing cells with 500 ng/ml α-IgM antibody resulted in Ca²⁺ oscillations with similar frequency to cells expressing S2−EE (FIG. 24B and pooled data C-E). However, the latency before the initiation of an increase in Ca²⁺ was significantly shorter in S2−EE-expressing cells. In addition, the magnitude of the initial transient was also significantly larger in S2−EE-expressing cells when compared with WT. Stimulation with 1 μg/ml α-IgM antibody resulted in transients that were indistinguishable in terms of latency, frequency, or initial peak magnitude in S2−EE- or S2−WT expressing cells (pooled data FIG. 24, C-E). Thus, PKA-mediated phosphorylation, by increasing the sensitivity of the InsP₃R-1 to InsP₃, can define the threshold at which cells begin to oscillate but does not markedly influence the temporal properties of Ca²⁺ oscillations when initiated. This latter observation presumably reflects the fact that the frequency of oscillations is primarily defined by mechanisms such as Ca²⁺ feedback (109) rather than the absolute sensitivity of the InsP₃R-1 to InsP₃ within a defined range. These data are largely consistent with data from hepatocytes where PKA activation resulted in cells exhibiting a lower threshold for activation by InsP₃ infusion (110) or agonist activation (111). These data using phosphomimetic mutations of InsP3R-1 splice variants are in broad agreement with a number of studies that have reported increased sensitivity of InsP₃R-1 activity following phosphoregulation by PKA (23, 24, 26, 100, 111, 112). An important consequence of this increased sensitivity is defining the threshold where a cell will respond to a stimulus. Given the almost ubiquitous expression of various forms of InsP₃R-1, this is likely a generally important phenomenon. A number of mechanisms are plausible to explain the increased sensitivity of InsP₃-induced Ca²⁺ release. These include modulation of InSP₃ binding, an idea supported by measurements of InsP₃R binding in hepatocytes (112) (presumably S2− InsP₃R-1 and InsP₃R-2). In these studies the apparent affinity of InsP₃ binding was enhanced ˜2-fold at resting [Ca²⁺], and the [Ca²⁺] necessary for half-maximal stimulation of InsP₃ binding was reduced. In contrast, a study of recombinant S1−S2+InsP₃R-1 expressed in SF9 cells and reconstituted into lipid bilayers has reported a similar increase in InsP₃R sensitivity to InsP₃ but that the bell-shaped Ca²⁺ sensitivity of channel opening is not altered following phosphorylation of the InsP₃R-1 (23). These findings indicate that modulation of the Ca²⁺ sensitivity of channel activity is unlikely to account for the increased apparent sensitivity of the receptor, at least in this form of the InsP₃R-1. There are a number of alternative mechanisms for altering InsP₃R-1 sensitivity are conceivable. For example, phosphorylation of the receptor could alter the gating of the channel directly. In addition, phosphorylation might secondarily modulate the receptor by regulation of the association of regulatory factors such as proteins or adenine nucleotides (113-115). Indeed, a precedent for this type of regulation exists because PKA phosphorylation has been shown to alter the association of calmodulin with the S2− form of InsP₃R-1 (106).

### Example 5

### InSP₃ Receptor Function

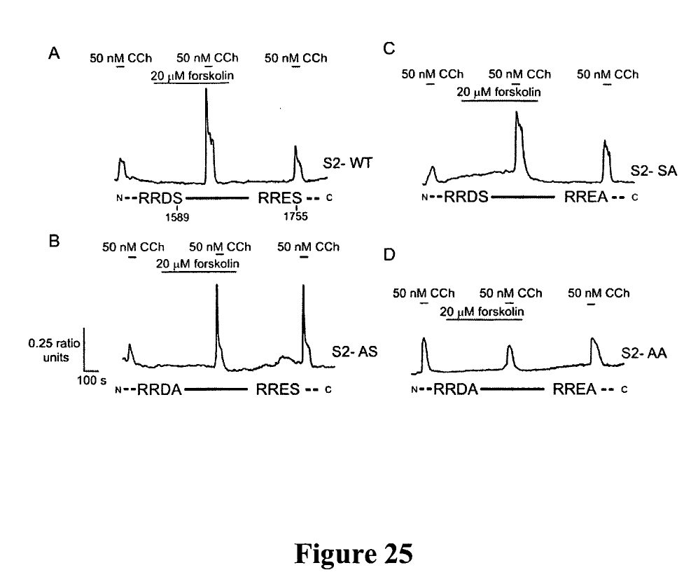

While both InsP₃R-1 and InsP₃-2 phosphorylation can result in enhanced Ca²⁺ release, studies have focused on the InsP₃R-1, in particular in defining which sites are functionally important in the splice variant of the InsP₃R-1 (S2⁻ variant) expressed in parotid acinar cells. Dt-403ko cells were transfected with S2⁻ InsP₃R-1 and muscarinic m3 receptors (m3R), along with red fluorescent protein (Hc-Red) to identify transfected cells. After loading with fura-2, treatment with forskolin was shown to result in a potentiated Ca²⁺ release in response to low concentrations of CCh (FIG. 25A) in a similar fashion to that observed in parotid acinar cells. Cells not transfected with either m3R or InsP₃R cDNA failed to respond. Similarly, cells not expressing Hc-Red seldom responded to CCh treatment.

Since Dt-403ko cells are a genetically tractable system, this has allowed a mutagenic analysis of the specific sites important for PKA phosphoregulation in the S2⁻ InsP₃R-1. Mutation of either potential phosphorylation site at serine 1589 or serine 1755 failed to attenuate this potentiation (FIGS. 25B and 25C respectively). In contrast, mutation of both serine residues to alanine (“double alanine” or S2−AA mutation) completely abolished potentiation of the CCh-induced Ca²⁺ release by forskolin (FIG. 25D). These data indicate that both consensus motifs in the S2⁻ InsP₃R-1 are subject to PKA-dependent phosphoregulation, an observation which is in contrast to the neuronal isoform of the InsP₃R-I where only S1755is subject to phosphoregulation. These experiments indicate that S1589 and S1755 constitute the only functional important PKA phosphorylation sites in S2⁻InsP₃R-2.

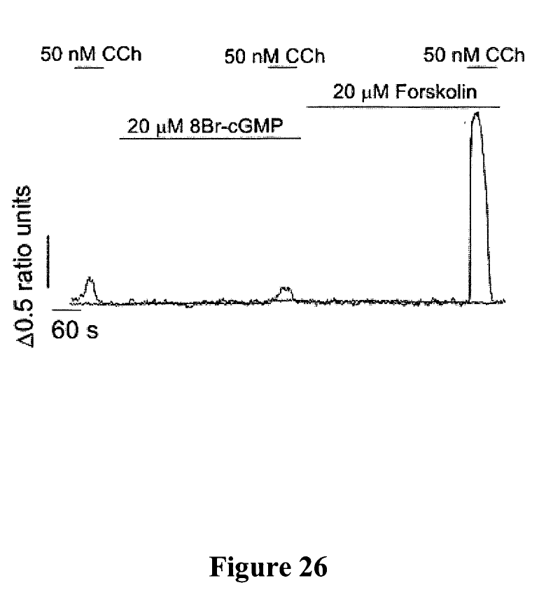

Because of its relative abundance in salivary tissue, and its high sensitivity to InsP₃, the InsP₃R-2 can be of key importance to salivary gland physiology. However, in the InsP₃ receptor field in general, only very limited progress regarding the unique structure and function of the InsP₃R-2 has been made. This is largely due to the difficulty of expressing this receptor subtype in a heterologous system. This problem was overcome by deletion of the 3′ and 5′ untranslated regions of the cDNA, and by inserting a kozak consensus sequence at the initiation site. As shown in FIG. 26, these manipulations have allowed for the successful expression of the optimized rat InsP₃R-2 in DT-403ko cells and, for the first time, to assess the effects of phosphorylation on an unambiguous homogeneous population of InsP₃R-2. Using a similar paradigm to that described for InsP₃R-1 (see FIG. 25) forskolin treatment results in a dramatic potentiation of carbachol-induced [Ca²⁺]ᵢ signaling. Interestingly activation of the cGMP dependent protein kinase with 8Br-cGMP is without effect. Mutation of the consensus phosphorylation sites in the receptor can allow for the development of similar phosphomimetic and non-phosphorylatable mutants in the InsP₃R-2.

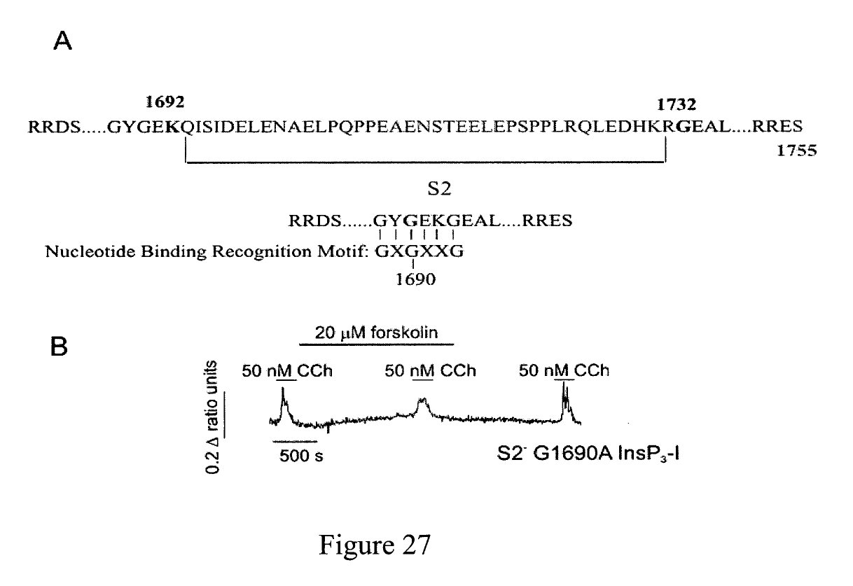

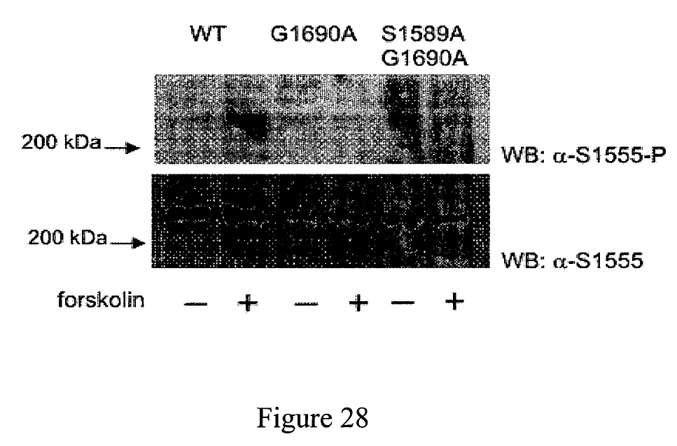

Significant progress has been made in probing the mechanism responsible for the effects of phosphorylation on the InsP₃R-1. Herein, it was noted that a potential mechanism for changing the apparent sensitivity of the InsP₃R following phosphorylation could be through alteration of the allosteric regulation of the receptor by ATP. Interestingly, it is also known that a unique nucleotide binding motif was created by the removal of 40 amino acids in the S2− (non-neuronal) splice variant of the InsP₃R-1 which was not present in the S2+ (neuronal) InsP₃R-1 (see FIG. 27A). A mutation in this nucleotide binding site was created by altering a critical glycine residue to alanine. As shown in FIG. 27B, when this construct was expressed in DT-403ko cells the signature PKA-dependent potentiation of the carbachol-induced Ca²⁺ elevation was strikingly absent (compare with FIG. 25A). However, a full concentration-response curve (generated in the same fashion to that illustrated in FIG. 18) demonstrated that this construct was still capable of producing a maximal response comparable to that seen in the wild-type receptor—only the sensitivity to phosphorylation was altered. The absence of PKA-mediated potentiation occurs because this construct was now not susceptible to phosphorylation in the absence of ATP binding as shown in FIG. 28. These data also indicate that the degree of binding of ATP, possibly reflecting the metabolic status of the cell, can tune the sensitivity of the receptor to the effects of PKA phosphorylation.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Berridge, M. J. (1993) Nature. 361(6410), 315-25
2. Furuichi, T., S. Yoshikawa, A. Miyawaki, K. Wada, N. Maeda, and K. Mikoshiba. (1989) Nature. 342(6245), 32-8
3. Furuichi, T., S. Yoshikawa, and K. Mikoshiba. (1989) Nucleic Acids Res. 17(13), 5385-6
4. Sudhof, T. C., C. L. Newton, B. T. Archer, 3rd, Y. A. Ushkaryov, and G. A. Mignery. (1991) Embo J. 10(11), 3199-206
5. Maranto, A. R. (1994) J Biol Chem. 269(2), 1222-30
6. Nakagawa, T., H. Okano, T. Furuichi, J. Aruga, and K. Mikoshiba. (1991) Proc Natl Acad Sci U S A. 88(14), 6244-8
7. Danoff, S. K., C. D. Ferris, C. Donath, G. A. Fischer, S. Munemitsu, A. Ullrich, S. H. Snyder, and C. A. Ross. (1991) Proc Natl Acad Sci U S A. 88(7), 2951-5
8. Yoshikawa, F., M. Morita, T. Monkawa, T. Michikawa, T. Furuichi, and K. Mikoshiba. (1996) J Biol Chem. 271(30), 18277-84
9. Mignery, G. A. and T. C. Sudhof. (1990) Embo J. 9(12), 3893-8
10. Michikawa, T., H. Hamanaka, H. Otsu, A. Yamamoto, A. Miyawaki, T. Furuichi, Y. Tashiro, and K. Mikoshiba. (1994) J Biol Chem. 269(12), 9184-9
11. Boehning, D., D. O. Mak, J. K. Foskett, and S. K. Joseph. (2001) J Biol Chem. 276(17), 13509-12
12. Patel, S., S. K. Joseph, and A. P. Thomas. (1999) Cell Calcium. 25(3), 247-64
13. Berridge, M. J., P. Lipp, and M. D. Bootman. (2000) Nat Rev Mol Cell Biol. 1(1), 11-21
14. Bootman, M. D., T. J. Collins, C. M. Peppiatt, L. S. Prothero, L. MacKenzie, P. De Smet, M. Travers, S. C. Tovey, J. T. Seo, M. J. Berridge, F. Ciccolini, and P. Lipp. (2001) Semin Cell Dev Biol. 12(1), 3-10
15. Mignery, G. A., P. A. Johnston, and T. C. Sudhof. (1992) J Biol Chem. 267(11), 7450-5
16. Bezprozvanny, I., J. Watras, and B. E. Ehrlich. (1991) Nature. 351(6329), 751-4
17. Iino, M. (1990) J Gen Physiol. 95(6), 1103-122
18. Iino, M. (1991) J Gen Physiol. 98(4), 681-98
19. Ehrlich, B. E. and J. Watras. (1988) Nature. 336(6199), 583-6
20. Yamada, M., A. Miyawaki, K. Saito, T. Nakajima, M. Yamamoto-Hino, Y. Ryo, T. Furuichi, and K. Mikoshiba. (1995) Biochem J. 308 (Pt 1), 83-8
21. Tertyshnikova, S., X. Yan, and A. Fein. (1998) J Physiol. 512 (Pt 1), 89-96
22. Tertyshnikova, S. and A. Fein. (1998) Proc Natl Acad Sci U S A. 95(4), 1613-7
23. Tang, T. S., H. Tu, Z. Wang, and I. Bezprozvanny. (2003) J Neurosci. 23(2), 403-115
24. Nakade, S., S. K. Rhee, H. Hamanaka, and K. Mikoshiba. (1994) J Biol Chem. 269(9), 6735-42
25. Supattapone, S., S. K. Danoff, A. Theibert, S. K. Joseph, J. Steiner, and S. H. Snyder. (1988) Proc Natl Acad Sci U S A. 85(22), 8747-50
26. Wojcikiewicz, R. J. and S. G. Luo. (1998) J Biol Chem. 273(10), 5670-7
27. Jayaraman, T., K. Ondrias, E. Ondriasova, and A. R. Marks. (1996) Science. 272(5267), 1492-4
28. Giovannucci, D. R., G. E. Groblewski, J. Sneyd, and D. I. Yule. (2000) J Biol Chem. 275(43), 33704-11
29. Bruce, J. I., T. J. Shuttleworth, D. R. Giovannucci, and D. I. Yule. (2002) J Biol Chem. 277(2), 1340-8
30. Joseph, S. K. and S. V. Ryan. (1993) J Biol Chem. 268(31), 23059-65
31. Ferris, C. D., R. L. Huganir, D. S. Bredt, A. M. Cameron, and S. H. Snyder. (1991) Proc Natl Acad Sci U S A. 88(6), 2232-5
32. den Dekker, E., J. W. Heemskerk, G. Gorter, H. van der Vuurst, J. Donath, C. Kroner, K. Mikoshiba, and J. W. Akkerman. (2002) Arterioscler Thromb Vasc Biol. 22(1), 179-86
33. DeSouza, N., S. Reiken, K. Ondrias, Y. M. Yang, S. Matkovich, and A. R. Marks. (2002) J Biol Chem. 277(42), 39397-400
34. Ferris, C. D., A. M. Cameron, D. S. Bredt, R. L. Huganir, and S. H. Snyder. (1991) Biochem Biophys Res Commun. 175(1), 192-8
35. Haug, L. S., V. Jensen, 0. Hvalby, S. I. Walaas, and A. C. Ostvold. (1999) J Biol Chem. 274(11), 7467-73
36. Komalavilas, P. and T. M. Lincoln. (1994) J Biol Chem. 269(12), 8701-7
37. Komalavilas, P. and T. M. Lincoln. (1996) J Biol Chem. 271(36), 21933-8
38. LeBeau, A. P., D. I. Yule, G. E. Groblewski, and J. Sneyd. (1999) J Gen Physiol. 113(6), 851-72
39. Pieper, A. A., D. J. Brat, E. O'Heam, D. K. Krug, A. I. Kaplin, K. Takahashi, J. H. Greenberg, D. Ginty, M. E. Molliver, and S. H. Snyder. (2001) Neuroscience. 102(2), 433-44
40. Koga, T., Y. Yoshida, J. Q. Cai, M. O. Islam, and S. Imai. (1994) J Biol Chem. 269(15), 11640-7
41. Straub, S. V., D. R. Giovannucci, J. I. Bruce, and D. I. Yule. (2002) J Biol Chem. 277(35), 31949-56
42. Miyakawa, T., A. Maeda, T. Yamazawa, K. Hirose, T. Kurosaki, and M. Iino. (1999) Embo J. 18(5), 1303-8
43. Sugawara, H., M. Kurosaki, M. Takata, and T. Kurosaki. (1997) Embo J. 16(11), 3078-88
44. Kubista, H., T. Hawkins, and S. E. Moss. (1998) Biochim Biophys Acta. 1448(2), 299-310
45. Li, W., J. Llopis, M. Whitney, G. Zlokamik, and R. Y. Tsien. (1998) Nature. 392(6679), 936-41
46. Cavallini, L., M. Coassin, A. Borean, and A. Alexandre. (1996) J Biol Chem. 271(10), 5545-51
47. Wojcikiewicz, R. J. (1995) J Biol Chem. 270(19), 11678-83
48. Rapoport, R. M., M. B. Draznin, and F. Murad. (1982) Proc Natl Acad Sci U S A. 79(21), 6470-4
49. Colbran, J. L., S. H. Francis, A. B. Leach, M. K. Thomas, H. Jiang, L. M. McAllister, and J. D. Corbin. (1992) J Biol Chem. 267(14), 9589-94
50. Houslay, M. D. and G. Milligan. (1997) Trends Biochem Sci. 22(6), 217-24
51. Ammendola, A., A. Geiselhoringer, F. Hofmann, and J. Schlossmann. (2001) J Biol Chem. 276(26), 24153-9
52. Schlossmann, J., A. Ammendola, K. Ashman, X. Zong, A. Huber, G. Neubauer, G. X. Wang, H. D. Allescher, M. Korth, M. Wilm, F. Hofmann, and P. Ruth. (2000) Nature. 404(6774), 197-201
53. Nezu, A., et al., Evidence that zymogen granules do not function as an intracellular Ca2+ store for the generation of the Ca2+ signal in rat parotid acinar cells. Biochem J, 2002. 363(Pt 1): p. 59-66.
54. Hassoni, A. A. and P. T. Gray, Flash photolysis studies of the localization of calcium release sites in rat parotid isolated acinar cells. J Physiol, 1994. 478 Pt 3: p. 461-7.
55. Sneyd, J., et al., A model of calcium waves in pancreatic and parotid acinar cells. Biophys J, 2003. 85(3): p. 1392-405.
56. Bruce, J. I., S. V. Straub, and D. I. Yule, Crosstalk between cAMP and Ca(2+) signaling in non-excitable cells. Cell Calcium, 2003. 34(6): p. 431-44.
57. Bruce, J. I., D. I. Yule, and T. J. Shuttleworth, Ca2+-dependent protein kinase—α modulation of the plasma membrane Ca2+-ATPase in parotid acinar cells. J Biol Chem, 2002. 277(50): p. 48172-81.
58. Mertz, L. M., B. J. Baum, and I. S. Ambudkar, Refill status of the agonist-sensitive Ca2+ pool regulates Mn2+ influx into parotid acini. 1990. 265(25): p. 15010-15014.
59. Putney, J. W., Jr., Biphasic modulation of potassium release in rat parotid gland by carbachol and phenylephrine. 1976. 198(2): p. 375-384.
60. Mertz, L. M., et al., Calcium entry in rat parotid acini: activation by carbachol and aluminum fluoride. 1990. 258(4 Pt 1): p. C654-C661.
61. Turner, R. J., Mechanisms of fluid secretion by salivary glands. Ann N Y Acad Sci, 1993. 694: p. 24-35.
62. Mandel, I. D., The role of saliva in maintaining oral homeostasis. J Am Dent Assoc, 1989. 119(2): p. 298-304.
63. Atkinson, J. C. and P. C. Fox, Salivary gland dysfunction. Clin Geriatr Med, 1992. 8(3): p. 499-511.
64. Baum, B. J., Principles of saliva secretion. Ann N Y Acad Sci, 1993. 694: p. 17-23.
65. Petersen, O. H., Stimulus-secretion coupling: cytoplasm calcium signals and the control of ion channels in exocrine acinar cells. J Physiol, 1992. 448: p. 1-51.
66. Frizzell, R. A., M. Field, and S. G. Schultz, Sodium-coupled chloride transport by epithelial tissues. Am J Physiol, 1979. 236(1): p. F1-8.
67. Melvin, J. E., Chloride channels and salivary gland function. Crt Rev Oral Biol Med, 1999. 10(2): p. 199-209.
68. Annotate, B., Regulation of electrolyte and fluid secretion in salivary acinar cells. Am J Physiol, 1992. 263(6 Pt 1): p. G823-37.
69. Evans, R. L., et al., Severe impairment of salivation in Na+/K+/2C1− co transporter (Nickel)-deficient mice. J Biol Chem, 2000. 275(35): p. 26720-6.
70. Melvin, J. E. and R. J. Turner, Cl− fluxes related to fluid secretion by the rat parotid: involvement of Cl(−)-HCO3-exchange. Am J Physiol, 1992. 262(3 Pt 1): p. G393-8.
71. Turner, R. J. and J. N. George, Cl(−)-HCO3-exchange is present with Na+-K+-Cl− cotransport in rabbit parotid acinar basolateral membranes. Am J Physiol, 1988. 254(3 Pt 1): p. C391-6.
72. Turner, R. J., J. N. George, and B. J. Baum, Evidence for a Na+/K+/Cl− cotransport system in basolateral membrane vesicles from the rabbit parotid. J Membr Biol, 1986. 94(2): p. 143-52.
73. Garrett, J. R. and A. Kidd, The innervation of salivary glands as revealed by morphological methods. Microsc Res Tech, 1993. 26(1): p. 75-91.
74. Tojyo, Y., A. Tanimura, and Y. Matsumoto, Imaging of intracellular Ca2+ waves induced by muscarinic receptor stimulation in rat parotid acinar cells. Cell Calcium, 1997. 22(6): p. 455-62.
75. Watson, E. L., et al., Identification of muscarinic receptor subtypes in mouse parotid gland. Am J Physiol, 1996. 271(3 Pt 1): p. C905-13.
76. Watson, E. L., J. A. Williams, and I. A. Siegel, Calcium mediation of cholinergic-stimulated amylase release from mouse parotid gland. Am J Physiol, 1979. 236(5): p. C233-7.
77. Awake, K., et al., Involvement of G alpha q/11 in m3-muscarinic receptor stimulation of phosphatidylinositol 4,5 bisphosphate-specific phospholipase C in rat parotid gland membranes. Arch Biochem Biophys, 1993. 305(2): p. 546-50.
78. Evans, R. L. and R. J. Turner, Upregulation of Na(+)-K(+)-2Cl− cotransporter activity in rat parotid acinar cells by muscarinic stimulation. J Physiol, 1997. 499 (Pt 2): p. 351-9.
79. Ambudkar, I. S., Regulation of calcium in salivary gland secretion. 2000. 11(1): p. 4-25.
80. Giovannucci, D. R., et al., Cytosolic Ca(2+) and Ca(2+)-activated Cl(−) current dynamics: insights from two functionally distinct mouse exocrine cells. J Physiol, 2002. 540(Pt 2): p. 469-84.
81. Zhang, X., et al., Ryanodine and inositol trisphosphate receptors are differentially distributed and expressed in rat parotid gland. Biochem J, 1999. 340 (Pt 2): p. 519-27.
82. Lee, M. G., et al., Polarized expression of Ca2+ channels in pancreatic and salivary gland cells. Correlation with initiation and propagation of [Ca2+]i waves. J Biol Chem, 1997. 272(25): p. 15765-70.
83. Tanimura, A., et al., Isoproterenol potentiates alpha-adrenergic and muscarinic receptor-mediated Ca2+ response in rat parotid cells. Am J Physiol, 1999. 276(6 Pt 1): p. C1282-7.
84. Tanimura, A., Y. Matsumoto, and Y. Tojyo, Evidence that isoproterenol-induced Ca2(+)-mobilization in rat parotid acinar cells is not mediated by activation of beta-adrenoceptors. Biochim Biophys Acta, 1990. 1055(3): p. 273-7.
85. Mak, D. O. and J. K. Foskett, Single-channel inositol 1,4,5-trisphosphate receptor currents revealed by patch clamp of isolated Xenopus oocyte nuclei. J Biol Chem, 1994. 269(47): p. 29375-8.
86. Mak, D. O., S. McBride, and J. K. Foskett, ATP regulation of type 1 inositol 1,4,5-trisphosphate receptor channel gating by allosteric tuning of Ca(2+) activation. J Biol Chem, 1999. 274(32): p. 22231-7.
87. Mak, D. O., et al., Single-channel properties in endoplasmic reticulum membrane of recombinant type 3 inositol trisphosphate receptor. J Gen Physiol, 2000. 115(3): p. 241-56.
88. Boehning, D., et al., Single-channel recordings of recombinant inositol trisphosphate receptors in mammalian nuclear envelope. Biophys J, 2001. 81(1): p. 117-24.
89. Mak, D. O., S. McBride, and J. K. Foskett, Regulation by Ca2+ and inositol 1,4,5-trisphosphate (InsP3) of single recombinant type 3 InsP3 receptor channels. Ca2+ activation uniquely distinguishes types 1 and 3 InsP3 receptors. J Gen Physiol, 2001. 117(5): p. 435-46.
90. Nakagawa, T., Shiota, C., Okano, H., and Mikoshiba, K. (1991) J Neurochem. 57, 1807-1810
91. Taylor, C. W., Genazzani, A. A., and Morris, S. A. (1999) Cell Calcium 26, 237-
92. Joseph, S. K., Boehning, D., Pierson, S., and Nicchitta, C. V. (1997) J Biol. Chem. 272, 1579-1588
93. Galvan, D. L., Borrego-Diaz, E., Perez, P. J., and Mignery, G. A. (1999) J. Biol. Chem. 274, 29483-29492
94. Yoshikawa, F., Iwasaki, H., Michikawa, T., Furuichi, T., and Mikoshiba, K. (1999) J. Biol. Chem. 274, 328-334
95. Ramos-Franco, J., Galvan, D., Mignery, G. A., and Fill, M. (1999) J. Gen. Physiol 114, 243-250
96. Galvan, D. L., and Mignery, G. A. (2002) J. Biol. Chem. 277, 48248-48260 16. Parker, A. K., Gergely, F. V., and Taylor, C. W. (2004) J. Biol. Chem. 279, 23797-23805
97. Parker, A. K., Gergely, F. V., and Taylor, C. W. (2004) J. Biol. Chem. 279, 23797-23805
98. Taylor, C. W., and Laude, A. J. (2002) Cell Calcium 32, 321-334
99. Soulsby, M. D., Alzayady, K., Xu, Q., and Wojcikiewicz, R. J. (2004) FEBS Lett. 557, 181-184
100. Wagner, L. E., II, Li, W. H., and Yule, D. I. (2003) J. Biol. Chem. 278, 45811-45817
101. Kennelly, P. J., and Krebs, E. G. (1991) J. Biol. Chem. 266, 15555-15558
102. Tateyarna, M., Rivolta, I., Clancy, C. E., and Kass, R. S. (2003) J. Biol. Chem. 278, 46718-46726
103. Reiken, S., Lacampagne, A., Zhou, H., Kherani, A., Lehnart, S. E., Ward, C., Huang, F., Gabuijakova, M., Gabuijakova, J., Rosemblit, N., Warren, M. S., He, K. L., Yi, G. H., Wang, J., Burkhoff, D., Vassort, G., and Marks, A. R. (2003) J. Cell Biol. 160, 919-928
104. Tu, H., Miyakawa, T., Wang, Z., Glouchankova, L., Iino, M., and Bezprozvanny, I. (2002) Biophys. J 82, 1995-2004
105. Boehning, D., and Joseph, S. K. (2000) J. Biol. Chem. 275, 21492-21499
106. Lin, C., Widjaja, J., and Joseph, S. K. (2000) J. Biol. Chem. 275, 2305-2311
107. Tu, H., Tang, T. S., Wang, Z., and Bezprozvanny, I. (2004) J. Biol. Chem. 279, 19375-19382
108. Berridge, M. J., Lipp, P., and Bootman, M. D. (2000) Nat. Rev. Mol. Cell Biol. 1, 11-21
109. Sneyd, J., Tsaneva-Atanasova, K., Yule, D. I., Thompson, J. L., and Shuttleworth, T. J. (2004) Proc. Natl. Acad. Sci. U. S A. 101, 1392-1396
110. Chatton, J. Y., Cao, Y., Liu, H., and Stucki, J. W. (1998) Biochem. J. 330, 1411-1416
111. Hajnoczky, G., Gao, E., Nomura, T., Hoek, J. B., and Thomas, A. P. (1993) Biochem. J. 293, 413-422
112. Joseph, S. K., and Ryan, S. V. (1993) J. Biol. Chem. 268, 23059-23065
113. Ando, H., Mizutani, A., Matsu-ura, T., and Mikoshiba, K. (2003) J. Biol. Chem. 278, 10602-10612
114. Boehning, D., Patterson, R. L., Sedaghat, L., Glebova, N. O., Kurosaki, T., and Snyder, S. H. (2003) Nat. Cell Biol. 5, 1051-1061
115. Maximov, A., Tang, T. S., and Bezprozvanny, I. (2003) Mol. Cell. Neurosci. 22, 271-283

What is claimed is:

1. A Inositol 1,4,5-trisphosphate receptor (InsP₃R) mutant, comprising at least one substitution of serine with a negatively charged amino acid residue at a phosphorylation site of a wild-type InsP₃R, wherein the mutant has an enhanced Ca²⁺ release function as compared to the wild-type InsP₃R.

2. The mutant of claim 1, wherein the Ca²⁺ release function is at least 5 times greater than the Ca²⁺ release function of the wild-type InsP₃R.

3. The mutant of claim 1, wherein the InsP₃R mutant is an InsP₃R-1 mutant and the wild-type InsP₃R is InsP₃R-1.

4. The mutant of claim 3, comprising at least one substitution of serine with a negatively charged amino acid residue at a phosphorylation site, wherein the phosphorylation site is selected from residue 1589 or 1755 of a wild-type InsP₃R-1 sequence.

5. The mutant of claim 4, wherein the substitution of serine with the negatively charged amino acid is at residue 1589.

6. The mutant of claim 4, wherein glutamate is substituted for serine at residue 1589.

7. The mutant of claim 6, wherein the mutant comprises the amino acid sequence of SEQ ID NO: 1.

8. The mutant of claim 6, wherein the mutant comprises the amino acid sequence of SEQ ID NO: 1 with one or more conservative amino acid substitutions.

9. The mutant of claim 6, wherein the mutant comprises the amino acid sequence of SEQ ID NO:2.

10. The mutant of claim 6, wherein the mutant comprises the amino acid sequence of SEQ ID NO:2 with one or more conservative amino acid substitutions.

11. The mutant of claim 4, wherein aspartate is substituted for serine at residue 1589.

12. The mutant of claim 11, wherein the mutant comprises the amino acid sequence of SEQ ID NO:3.

13. The mutant of claim 11, wherein the mutant comprises the amino acid sequence of SEQ ID NO:3 with one or more conservative amino acid substitutions.

14. The mutant of claim 11, wherein the mutant comprises the amino acid sequence of SEQ ID NO:4.

15. The mutant of claim 11, wherein the mutant comprises the amino acid sequence of SEQ ID NO:4 with one or more conservative amino acid substitutions.

16. The mutant of claim 4, wherein the substitution of serine with the negatively charged amino acid is at residue 1755.

17. The mutant of claim 16, wherein glutamate is substituted for serine at residue 1755.

18. The mutant of claim 17, wherein the mutant comprises the amino acid sequence of SEQ ID NO:5.

19. The mutant of claim 17, wherein the mutant comprises the amino acid sequence of SEQ ID NO:5 with one or more conservative amino acid substitutions.

20. The mutant of claim 17, wherein the mutant comprises the amino acid sequence of SEQ ID NO:6.

21. The mutant of claim 17, wherein the mutant comprises the amino acid sequence of SEQ ID NO:6 with one or more conservative amino acid substitutions.

22. The mutant of claim 16, wherein aspartate is substituted for serine at residue 1755.

23. The mutant of claim 22, wherein the mutant comprises the amino acid sequence of SEQ ID NO:7.

24. The mutant of claim 22, wherein the mutant comprises the amino acid sequence of SEQ ID NO:7 with one or more conservative amino acid substitutions.

25. The mutant of claim 22, wherein the mutant comprises the amino acid sequence of SEQ ID NO:8.

26. The mutant of claim 22, wherein the mutant comprises the amino acid sequence of SEQ ID NO:8 with one or more conservative amino acid substitutions.

27. The mutant of claim 4, wherein the substitutions of serine with the negatively charged amino acid is at residues 1589 and 1755.

28. The mutant of claim 27, wherein glutamate is substituted for serine at residues 1589 and 1755.

29. The mutant of claim 28, wherein the mutant comprises the amino acid sequence of SEQ ID NO:9.

30. The mutant of claim 28, wherein the mutant comprises the amino acid sequence of SEQ ID NO:9 with one or more conservative amino acid substitutions.

31. The mutant of claim 28, wherein the mutant comprises the amino acid sequence of SEQ ID NO:10.

32. The mutant of claim 28, wherein the mutant comprises the amino acid sequence of SEQ ID NO:10 with one or more conservative amino acid substitutions.

33. The mutant of claim 27, wherein aspartate is substituted for serine at residues 1589 and 1755.

34. The mutant of claim 33, wherein the mutant comprises the amino acid sequence of SEQ ID NO: 11.

35. The mutant of claim 33, wherein the mutant comprises the amino acid sequence of SEQ ID NO: 11 with one or more conservative amino acid substitutions.

36. The mutant of claim 33, wherein the mutant comprises the amino acid sequence of SEQ ID NO: 12.

37. The mutant of claim 33, wherein the mutant comprises the amino acid sequence of SEQ ID NO: 12 with one or more conservative amino acid substitutions.

38. The mutant of claim 27, wherein aspartate is substituted for serine at residue 1589 and glutamate is substituted for serine at residue 1755.

39. The mutant of claim 37, wherein the mutant comprises the amino acid sequence of SEQ ID NO:13.

40. The mutant of claim 37, wherein the mutant comprises the amino acid sequence of SEQ ID NO: 13 with one or more conservative amino acid substitutions.

41. The mutant of claim 37, wherein the mutant comprises the amino acid sequence of SEQ ID NO:14.

42. The mutant of claim 37, wherein the mutant comprises the amino acid sequence of SEQ ID NO:14 with one or more conservative amino acid substitutions.

43. The mutant of claim 25, wherein glutamate is substituted for serine at residue 1589 and aspartate is substituted for serine at residue 1755.

44. The mutant of claim 43, wherein the mutant comprises the amino acid sequence of SEQ ID NO: 15.

45. The mutant of claim 43, wherein the mutant comprises the amino acid sequence of SEQ ID NO: 15 with one or more conservative amino acid substitutions.

46. The mutant of claim 43, wherein the mutant comprises the amino acid sequence of SEQ ID NO:16.

47. The mutant of claim 43, wherein the mutant comprises the amino acid sequence of SEQ ID NO: 16 with one or more conservative amino acid substitutions.

48. An InsP₃R mutant of an InsP₃R short-form splice variant, comprising a substitution of one or more glycines that form the binding motif of the binding site generated by the splice variation.

49. The mutant of claim 48, wherein the InsP₃R short form splice variant is InsP₃R-1 S2−.

50. The mutant of claim 48, further comprising a substitution at residue 1690.

51. The mutant of claim 50, wherein the substitution is a glycine to alanine substitution.

52. The mutant of claim 51, wherein the mutant comprises the amino acid sequence of SEQ ID NO: 23 with one or more conservative amino acid substitutions.

53. A nucleic acid that encodes the mutant of claim 1-52.

54. An expression vector comprising the nucleic acid of claim 53 operable linked to an expression control sequence.

55. A cultured cell comprising the vector of claim 53.

56. The cell of claim 55, wherein the cell is a DT-40 cell.

57. The cell of claim 56, wherein the cell further comprises a nucleic acid that encodes an acetylcholine receptor.

58. The cell of claim 57, wherein the acetylcholine receptor is an M3 receptor.

59. An InsP₃R mutant, comprising at least one substitution of serine with an amino acid with an aliphatic side chain at a phosphorylation site of a wild-type InsP₃R, wherein the mutant is nonphosphorylatable.

60. The mutant of claim 55, wherein the nonphosphorylatable mutant is an InsP₃R-1 mutant.

61. The mutant of claim 55, wherein the nonphosphorylatable mutant of InsP₃R is selected from the group consisting of an S1755A, or S1589A/S1755A mutation.

62. A nucleic acid that encodes the mutant of claim 55.

63. An expression vector comprising the nucleic acid of claim 62 operable linked to an expression control sequence.

64. A cultured cell comprising the vector of claim 62.

65. The cell of claim 64, wherein the cell is a DT-40 cell.

66. The cell of claim 65 wherein the cell further comprises a nucleic acid that encodes an acetylcholine receptor.

67. The cell of claim 66, wherein the acetylcholine receptor is an M3 receptor.

68. A method of screening for an agent that preferentially modulates Ca²⁺ release by phosphorylated InsP₃R, comprising
a. contacting the cell of claim 55 with the agent to be screened, under conditions that allow Ca²⁺ release;
b. measuring Ca²⁺ release; and
c. comparing the amount of Ca²⁺ release in step b with a control cell, wherein the control cell comprises an un-phosphorylated InsP₃R and wherein the control cell is contacted with the agent to be screened, an increase or decrease in Ca²⁺ release as compared to a control cell indicating an agent that preferentially modulates unphosphorylated InsP₃R.

69. The method of claim 68, wherein the un-phosphorylated InsP₃R is a nonphosphorylatable mutant InsP₃R.

70. The method of claim 69, wherein the nonphosphorylatable mutant comprises a substitution of a serine at a phosphoylation site with an amino acid having an aliphatic side-chain.

71. The method of claim 70, wherein the amino acid having an aliphatic side chain is alanine.

72. The method of claim 70, wherein the phosphorylaytion site is either residue 1589 or 1755 or a combination thereof of wild-type InsP₃R.

73. A method of expressing a mutant InsP₃R in a cell in vivo, comprising
a. providing the expression vector of claim 53;
b. introducing the vector into a cell in vivo;
c. maintaining the cell under condition that permit expression of the mutant InsP₃R by the cell.

74. A method of treating a subject with xerostomia, comprising introducing into the subject the expression vector of claim 53 under conditions that an amount of InsP₃R mutant is expressed in an effective amount to alleviate the symptoms of xerostomia.

75. A method of treating a subject with cystic fibrosis, comprising introducing into the subject the expression vector of claim 53 under conditions that an amount of InsP₃R mutant is expressed in an effective amount to alleviate the symptoms of cystic fibrosis.

76. The mutant of claim 1, wherein the InsP₃R mutant is an InsP₃R-2 mutant and the wild-type InsP₃R is InsP₃R-2.

77. The mutant of claim 76, comprising at least one substitution of serine with a negatively charged amino acid residue at a phosphorylation site, wherein the phosphorylation site is selected from residue 766, 1772, 1856, 2058, 2227 of a wild-type InsP₃R-2 sequence.

78. The mutant of claim 77, wherein one or more serines are substituted with glutamate.

79. The mutant of claim 77, wherein one or more serines are substituted with aspartate.

80. The mutant of claim 77, wherein any combination of the serines are substituted with any combination of aspartate or glutamate.

81. The mutant of claim 1, wherein the InsP₃R mutant is an InsP₃R-3 mutant and the wild-type InsP₃R is InsP₃R-3.

82. The mutant of claim 81, comprising at least one substitution of serine with a negatively charged amino acid residue at a phosphorylation site, wherein the phosphorylation site is selected from residue 934, 1640, 1834, 2009, 2041, 2189 of a wild-type InsP₃R-3 sequence.

83. The mutant of claim 82, wherein one or more serines are substituted with glutamate.

84. The mutant of claim 82, wherein one or more serines are substituted with aspartate.

85. The mutant of claim 82, wherein any combination of the serines are substituted with any combination of aspartate or glutamate.

86. A method of inhibiting apoptosis in a transplant in a subject comprising introducing into the transplant the expression vector of claim 53 under conditions that an amount of an InsP₃R mutant is expressed in an effective amount to inhibit cell death.

87. The method of claim 86, wherein the transplant comprises B cells.

88. A method of treating a subject with HIV, comprising introducing into the subject the expression vector of claim 53 under conditions that an amount of InsP₃R mutant is expressed in an effective amount to alleviate the symptoms of HIV.

89. A method of treating a subject with arthritis, comprising introducing into the subject the expression vector of claim 53 under conditions that an amount of InsP₃R mutant is expressed in an effective amount to alleviate the symptoms of arthritis.


SEQUENCE LISTING (32 sequences)

SEQ ID NO: 1 - protein, 2710 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDEVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKGEALRQILVNRYYGNIRPSGRRESLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 2 - protein, 2749 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDEVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDELENAELPQPPEAENSTEELEPSPPLRQLEDHKRGEALRQILVNRYYGNIRPSGRRESLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 3 - protein, 2710 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDDVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKGEALRQILVNRYYGNIRPSGRRESLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 4 - protein, 2749 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDDVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDELENAELPQPPEAENSTEELEPSPPLRQLEDHKRGEALRQILVNRYYGNIRPSGRRESLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 5 - protein, 2710 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKGEALRQILVNRYYGNIRPSGRREELTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 6 - protein, 2749 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDELENAELPQPPEAENSTEELEPSPPLRQLEDHKRGEALRQILVNRYYGNIRPSGRREELTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 7 - protein, 2710 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKGEALRQILVNRYYGNIRPSGRREDLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 8 - protein, 2749 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDELENAELPQPPEAENSTEELEPSPPLRQLEDHKRGEALRQILVNRYYGNIRPSGRREDLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 9 - protein, 2710 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDEVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKGEALRQILVNRYYGNIRPSGRREELTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 10 - protein, 2749 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDEVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDELENAELPQPPEAENSTEELEPSPPLRQLEDHKRGEALRQILVNRYYGNIRPSGRREELTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 11 - protein, 2710 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDDVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKGEALRQILVNRYYGNIRPSGRREDLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 12 - protein, 2749 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDDVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDELENAELPQPPEAENSTEELEPSPPLRQLEDHKRGEALRQILVNRYYGNIRPSGRREDLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 13 - protein, 2710 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDDVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKGEALRQILVNRYYGNIRPSGRREELTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 14 - protein, 2749 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDDVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDELENAELPQPPEAENSTEELEPSPPLRQLEDHKRGEALRQILVNRYYGNIRPSGRREELTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 15 - protein, 2710 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDEVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKGEALRQILVNRYYGNIRPSGRREDLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 16 - protein, 2749 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDEVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDELENAELPQPPEAENSTEELEPSPPLRQLEDHKRGEALRQILVNRYYGNIRPSGRREDLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 17 - protein, 2710 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKGEALRQILVNRYYGNIRPSGRRESLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 18 - protein, 2749 aa, Artificial Sequence
  Description of Artificial Sequence/note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDELENAELPQPPEAENSTEELEPSPPLRQLEDHKRGEALRQILVNRYYGNIRPSGRRESLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 19 - protein, 2701 aa, Artificial Sequence
  Description of Artificial Sequence/note = synthetic construct
MSDKMSSFLYIGDIVSLYAEGSVNGFISTLGLVDDRCVVHPEAGDLTNPPKKFRDCLFKVCPMNRYSAQKQYWKAKQAKQGNHTEAALLKKLQHAAELEQKQNESENRKLLGEIVKYSKVIQLLHIKSNKYLTVNKRLPALLEKNAMRVSLDAAGNEGSWFYIHPFWKLRSEGDNIVVGDKVVLMPVNAGQPLHASNVELLDNPGCKEVNAVNCNTSWKITLFMKFSSYREDVLKGGDVVRLFHAEQEKFLTCDDYEKKQHIFLRTTLRQSATSATSSKALWEIEVVHHDPCRGGAGQWNSLFRFKHLATGNYLAAELNPDYRDAQNEGKTVRDGELPTSKKKHQAGEKIMYTLVSVPHGNDIASLFELDATTLQRADCLVPRNSYVRLRHLCTNTWVTSTSIPIDTEEERPVMLKIGTCQTKEDKEAFAIVCVPLSEVRDLDFANDANKVLATTVKKLENGSITQNERRFVTKLLEDLIFFVADVTNNGQDVLDVVITKPNRERQKLMREQNILAQVFGILKAPFKEKAGEGSMLRLEDLGDQRYAPYKYVLRLCYRVLRHSQQDYRKNQEYIAKNFCVMQSQIGYDILAEDTITALLHNNRKLLEKHITAKEIETFVSLLRRNREPRFLDYLSDLCVSNSTAIPVTQELICKFMLSPGNADILIQTKLVSMQVENPMESSILPDDIDDEEVWLYWIDSNKEPHGKAIRHLAQEAREGTKADLEVLTYYRYQLNLFARMCLDRQYLAINQISTQLSVDLILRCVSDESLPFDLRASFCRLMLHMHVDRDPQESVVPVRYARLWTEIPTKITIHEYDSITDSSRNDMKRKFALTMEFVEEYLKEVVNQPFPFGDKEKNKLTFEVVHLARNLIYFGFYSFSELLRLTRTLLAILDIVQAPMSSYFERLSKFQDGSNNVMRTIHGVGEMMTQMVLSRGSIFPVSWPDAQPSVHPSKQASPGEQEDVTVMDTKLKVIEILQFILSVRLDYRISYMLSIYKKEFGENDGNGDPSASGTPETLLPSALVPDIDEIAAQAETMFAGRKEKTPVQLDDEGGRTFLRVLIHLIMHDYAPLLSGALQLLFKHFSQRAEVLQAFKQVQLLVSNQDVDNYKQIKADLDQLRLTVEKSELWVEKSGSYENGDMGEGQAKGGEEANEESNLLSPVQDGAKTPQIDSNKGNNYRIVKEILIRLSKLCVQNKKCRNQHQRLLKNMGAHSVVLDLLQIPYEKTDEKMNEVMDLAHTFLQNFCRGNPQNQVLLHKHLNLFLTPGLLEAETMRHIFMNNYHLCNEISERVVQHFVHCIETHGRHVEYLRFLQTIVKADGKYVKKCQDMVMTELINGGEDVLIFYNDRASFPILLNMMCSERARGDESGPLAYHITLVELLAACTEGKNVYTEIKCNSLLPLDDIVRVVTHDDCIPEVKIAYVNFVNHCYVDTEVEMKEIYTSNHIWKLFENFLVDMARVCNTTTDRKHADTFLERCVTESVMNIVSGFFNSPFSDNSTSLQTHQPVFIQLLQSAFRIYNCTWPNPAQKASVESCIRALAEVAKNRGIAIPVDLDSQVNTLFMKNHSSTVQRAAMGWRLSARSGPRFKEALGGPAWDYRNIIEKLQDVVASLEQQFSPMMQAEFSVLVDVLYSPELLFPEGSDARIRCGAFMSKLINHTKKLMEKEEKLCIKILQTLREMLEKKDSFMEESSTLRKILLNRYFKGDHSVGVNGPLSGAYAKTAQVGGGFTGQDADKTGISMSDIQCLLDKEGASELVIDVIVNTKNDRIFSEGILLGIALLEGGNTQTQNSFYQQLHEQKKSEKFFKVLYDRMKAAQKEIRSTVTVNTIDLGSKKREEDSDLMALGPRMRVRDSSLHLKEGMKGQLTEASSATSKAYCVYRREMDPDIDTMCPGQEAGSAEEKSAEEVTMSPAITIMRPILRFLQLLCENHNRELQNFLRNQNNKTNYNLVCETLQFLDCICGSTTGGLGLLGLYINEKNVALVNQTLESLTEYCQGPCHENQTCIATHESNGIDIIIALILSDINPLGKYRMDLVLQLKNNASKLLLAIMESRHDSENAERILFNMRPKELVDVMKNAYNQGLECNHGDEEGGDDGVSPKDVGHNIYILAHQLARHNKLLQQMLKPGSDPEEGDEALKYYANHTAQIEIVRHDRTMEQIVFPVPNICEFLTRESKYRVFNTTERDEQGSKVNDFFQQTEDLYNEMKWQKKIRNNPALFWFSRHISLWGSISFNLAVFINLAVALFYPFGDDGDEGTLSPLFSALLWVAVAICTSMLFFFSKPVGIRPFLVSIMLRSIYTIGLGPTLILLGAANLCNKIVFLVSFVGNRGTFTRGYRAVILDMAFLYHVAYVLVCMLGLFVHEFFYSFLLFDLVYREETLLNVIKSVTRNGRSIILTAVLALILVYLFSIIGFLFLKDDFTMEVDRLKNRTPVTGNDGVPTMTLTSMLGTCPKENCSPTIPSSNAAGEGGEDGIERTCDTLLMCIVTVLNQGLRNGGGVGDVLRRPSKDEPLFAARVVYDLLFFFIVIIIVLNLIFGVIIDTFADLRSEKQKKEKILKTTCFICGLERDKFDNKTVSFEEHIKSEHNMWHYLYFIVLVKVKDPTEYTGPESYVAQMITEKNLDWFPRMRAMSLVSNEGDSEQNEIRNLQEKLESTMSLVKQLSGQLAELKEQMTEQRKNKQRLGFLGSNTPHENHHMPPH

SEQ ID NO: 20 - protein, 2670 aa, Artificial Sequence
  Description of Artificial Sequence/note = synthetic construct
MNEMSSFLHIGDIVSLYAEGSVNGFISTLGLVDDRCVVEPAAGDLDNPPKKFRDCLFKVCPMNRYSAQKQYWKAKQTKQDKEKIADVVLLQKLQHAAQMEQKQNDTENKKVHGDVVKYGSVIQLLHMKSNKYLTVNKRLPALLEKNAMRVTLDATGNEGSWLFIQPFWKLRSNGDNVVVGDKVILNPVNAGQPLHASNYELSDNVGCKEVNSVNCNTSWKINLFMQFRDHLEEVLKGGDVVRLFHAEQEKFLTCDEYRGKLQVFLRTTLRQSATSATSSNALWEVEVVHHDPCRGGAGHWNGLYRFKHLATGNYLAAEENPSYKGDVSDPKAAGPGAQSRTGRRNAGEKIKYRLVAVPHGNDIASLFELDPTTLQKTDSFVPRNSYVRLRHLCTNTWIQSTNAPIDVEEERPIRLMLGTCPTKEDKEAFAIVSVPVSEIRDLDFANDASSMLASAVEKLNEGFISQNDRRFVIQLLEDLVFFVSDVPNNGQNVLDIMVTKPNRERQKLMRDENILKQIFGILKAPFRDKGGEGPLVRLEELSDQKNAPYQYMFRLCYRVLRHSQEDYRKNQEHIAKQFGMMQSQIGYDILAEDTITALLHNNRKLLEKHITKTEVETFVSLVRKNREPRFLDYLSDLCVSNRIAIPVTQELICKCVLDPKNSDILIQTELRPVKEMAQSHEYLSIEYSEEEVWLTWTDRNNEHHEKSVRQLAQEARAGNAHDENVLSYYRYQLKLFARMCLDRQYLAIDEISKQLGVELLFLCMADEMLPFDLRASFCHLMLHVHVDRDPQELVTPVKFARLWTEIPTAITIKDYDSNLNASRDDKKNKFASTMEFVEDYLNNVVGEAVPFANDEKNILTFEVVSLAHNLIYFGFYSFSELLRLTRTLLGIIDCIQAPAAVLQAYEEPGGKNVRRSIQGVGHMMSTMVLSRKQSVFGASSLPTGVGVPEQLDRSKFEDNEHTVVMETKLKILEILQFILNVRLDYRISYLLSVFKKEFVEVFPMQDSGADGTAPAFDSSTANMNLDRIGEQAEAMFGVGKTSSMLEVDDEGGRMFLRVLLHLTMHDYPPLVSGALQLLFKHFSQRQEAMHTFKQVQLLISAQDVENYKVIKSELDRLRTMVEKSELWVDKKGSVKGEEGEAGASKDKKERPSDEEGFLQPHGEKSSENYQIVKGILERLNKMCGVGEQMRKKQQRLLKNMDAHKVMLDLLQIPYDKNDNKMMEILRYTHQFLQKFCAGNPGNQALLHKHLQLFLTPGLLEAETMQHIFLNNYQLCSEISEPVLQHFVHCWPTHGRHVQYLDFLHTVIKAEGKYVKKCQDMIMTELTNAGDDVVVFYNDKASLAHLLDMMKAARDGVEDHSPLMYHISLVDLLAACAEGKNVYTEIKCTSLLPLEDVVSVVTHEDCITEVKMAYVNFVNHCYVDTEVEMKEIYTSNHIWTLFENFTLDMALVCNKREKRLSDPTLEKYVLTVVLDTISAFFSSPFSENSTSLQTHQTIVVQLLQSTTRLLECPWLQQQHKGSVEACVRTLAMVAKSRAILLPMDLDAHMSALLSSGGSCSAAAQRSAANYKTATRTFPRVIPTANQWDYKNIIEKLQDIITALEERLKPLVQAELSVLVDMLHWPELLFLEGSEAYQRCESGGFLSKLIRHTKGLMESEEKLCVKVLRTLQQMLQKKSKYGDRGNQLRKMLLQNYLQNRKSGPRGELTDPTGSGVDQDWSAIAATQCRLDKEGATKLVCDLITSTKNEKIFQESIGLAIRLLDGGNTEIQKSFYNLMTSDKKSERFFKVLHDRMKRAQQETKSTVAVNMSDLGSQPREDREPADPTTKGRVSSFSMPSSSRYSLGPGLHRGHDVSERAQNNEMGTSVLIMRPILRFLQLLCENHNRDLQNFLRCQNNKTNYNLVCETLQFLDIMCGSTTGGLGLLGLYINEDNVGLVIQTLETLTEYCQGPCHENQTCIVTHESNGIDIITALILNDISPLCKYRMDLVLQLKDNASKLLLALMESRHDSENAERILISLRPQELVDVIKKAYLQEEERENSEVSPREVGHNIYILALQLSRHNKQLQHLLKPVKRIQEEEAEGISSMLSLNNKQLSQMLKSSAPAQEEEEDPLAYYENHTSQIEIVRQDRSMEQIVFPVPAICQFLTEETKHRLFTTTEQDEQGSKVSDFFDQSSFLHNEMEWQRRLRSMPLIYWFSRRMTLWGSISFNLAVFINIIIAFFYPYVEGASTGVLGSPLISLLFWILICFSIAALFTKHYSVRPLIVALVLRSIYYLGIGPTLNILGALNLTNKIVFVVSFVGNRGTFIRGYKAMVMDMEFLYHVGYILTSVLGLFAHELFYSILLFDLIYREETLFNVIKSVTRNGRSILLTALLALILVYLFSIVGFLFLKDDFILEVDRLPGNHSRASTLGMPHGAATFMGTCSGDKMDCVSEVSVPEILEEDEELDSTERACDTLLMCIVTVMNHGLRNGGGVGDILRKPSKDESLFPARVVYDLLFFFIVIIIVLNLIFGVIIDTFADLRSEKQKKEEILKTTCFICGLERDKFDNKTVSFEEHIKLEHNMWNYLYFIVLVRVKNKTDYTGPESYVAQMIKNKNLDWFPRMRAMSLVSGEGEGEQNEIRILQEKLGSTMKLVSHLTAQLNELKEQMTEQRKRRQRLGFVDVQNCMSR

SEQ ID NO: 21 - protein, 4 aa, Artificial Sequence
  Description of Artificial Sequence/note = synthetic construct
RXXS

SEQ ID NO: 22 - DNA, 9759 bp, Artificial Sequence
  Description of Artificial Sequence/note = synthetic construct
gagagaaagcgcacgccgagaggaggtgtgggtgttccgctcccatcctaacggaacgagctccctcttcgcggacatgggattgcccagcggctgctaacccctgtcctggtcctgatccctcaaaccggcgtggctccccggtcaccaaggagctgactacaaaggatttgcatccttggctgggagtccattggctacagagtgcctgacctgggtcaggcttttcaacaaggacatgtctgacaaaatgtctagtttcctacatatcggagacatttgttctctgtatgcagagggatctacgaatggatttatcagcaccttgggcttggttgatgaccgttgcgttgtacagccagaagctggggaccttaacaatccacccaagaaattcagagactgcctctttaagctatgtcctatgaatcgatattctgcacagaagcagttctggaaagctgctaagcccggggccaacagtacaacagatgcggtgctgctcaacaaattgcatcatgctgcagacttggaaaagaagcagaatgagacagaaaacaggaaattgctggggacggtcatccagtatggcaacgtgatccagctcctacatttgaaaagcaataaatacttaactgtgaataagaggcttcctgccctgctggagaagaacgccatgagagtgacgttggatgaggctggaaatgaagggtcttggttttacattcagccgttctacaagctccgctccatcggagacagtgtggtcataggtgacaaggtagttttgaaccctgtcaatgctgggcagccgctacatgccagcagccatcagctggtggataacccgggctgcaatgaggtcaactccgtcaactgtaacacaagctggaaaatagtgcttttcatgaaatggagtgataacaaagatgacattctaaaaggaggtgatgtggtaagactcttccatgctgagcaagagaagtttctcacgtgcgatgagcacaggaagaagcagcacgtcttcctgaggacaaccggcagacagtcagccacgtcggccaccagctccaaagccctgtgggaagtggaggtagtccagcatgacccatgtcggggtggagctgggtactggaatagcctcttccggttcaagcacctggctacagggcattacttggcagcagaggtagaccctgactttgaggaagaatgcctggagtttcagccctcagtggaccctgatcaggacgcatctcggagcaggttgagaaatgcccaagaaaaaatggtgtactctctggtctctgtgcctgagggcaacgacatctcctccatctttgagctagaccccacaactctacgtggaggtgacagccttgtcccaaggaactcctatgtccggctcagacacctgtgtacaaacacctgggttcacagcacaaacatccccattgacaaggaagaggaaaaacctgtcatgctgaagattggtacctctcccctgaaggaggacaaggaagcttttgccatagtccccgtttctcctgctgaggttcgggacctggactttgccaatgatgccagcaaggtgctgggctccatcgctgggaagttggaaaagggcaccatcacccagaatgagagaaggtctgtcaccaagctgctggaagacttggtttactttgtcacgggtggaacgaactctggccaagacgtgcttgaagtagtcttctctaagcccaaccgagagcggcagaagctgatgagggaacagaatattctcaagcagatcttcaagcttttgcaggcccccttcacggactgcggggatggcccaatgcttcggctggaggagctgggggaccagcggcacgctcctttcagacacatttgccgactctgctacagggtcctgagacactcacagcaagactacaggaagaaccaggagtacatagccaagcagtttggcttcatgcagaagcagattggctatgatgtgctggccgaagacaccatcactgccctgctccacaacaaccgaaagctcctggaaaagcacatcaccgcagcagagattgacacgtttgtcagcctggtgcgaaagaacagggagcccaggttcttggattacctctctgacctctgtgtatccatgaataagtcaatccctgtgacccaggagctcatttgtaaagctgtgctgaatcccaccaatgctgacatcctgattgagaccaagttggttctttctcgttttgagtttgaaggcgtttccaccggagagaatgctctggaagctggggaagatgaggaagaggtgtggctgttctggagggacagcaacaaagagatccgtagcaagagtgttagggaattggcacaagatgctaaagagggacagaaggaagacagggatgtcctcagctactacaggtatcagctgaacctctttgcaaggatgtgtctggaccgccagtacctggccatcaatgaaatatccgggcagctggatgttgacctcatcctccgctgcatgtccgatgagaacctcccctatgacctcagagcatccttttgccgcctcatgcttcacatgcatgtggaccgagatccccaggagcaggtgacccctgtgaaatacgcccgtctgtggtcagaaattccctccgagatcgccattgacgactatgacagcagtggagcatcgaaggatgaaatcaaggagaggtttgctcagacgatggagtttgtggaggagtacctgagagatgtggtttgtcaaagattccccttttctgataaggagaaaaataagctcacatttgaggttgtgaacttagccaggaacctcatatactttggcttctacaacttttctgaccttctccgattaaccaagatcctcttggcaatcttagactgtgttcatgtgacaaccatcttccccattagcaagatgacgaaaggcgaagagaataaaggcagcaacgtgatgagatctatccatggagttggggagctcatgacccaggtggtgcttcggggaggaggctttttgcccatgactcctatggctgctgcccctgaaggaaatgtgaagcaggcagagccagagaaagaggatatcatggtcatggacaccaagttaaagatcattgaaatactccagtttattttgaatgtgagattggattataggatctcctgcctcctgtgtatatttaagcgggagtttgatgaaagcaattcccagtcatcagaaacatcctccggaaacagcagccaagaagggccaagtaatgtaccaggtgctcttgactttgaacacattgaagagcaagcagaaggcatctttgggggaagtgaggagaacacacctctggacttggatgatcatggtggcagaaccttcctcagggtcctgctccacttgacaatgcatgattacccacccctggtgtctggggccctgcagctcctcttccggcatttcagccagaggcaggaggtccttcaggccttcaaacaggttcaactgctggttactagccaagatgtggacaactacaaacagatcaagcaagacttggaccagctaagatccatcgtggagaagtctgagctctgggtatacaaaggccagggtcccgatgagcctatggacggagcctctggtgaaaacgaacataagaaaactgaggaggggaccagtaagccactgaagcacgaaagcaccagcagctacaactaccgagtggtgaaagagattctgattcgacttagcaagctctgtgttcaagagagcgcgtcggtgaggaagagcaggaagcagcagcagcgactgctgaggaacatgggtgcccatgctgtggtgctggagctgctgcagatcccctatgagaaggccgaagataccaagatgcaggagatcatgaggctggctcacgaatttctgcagaatttctgcgcaggcaaccagcagaaccaagctttgctacataaacacataaacctgtttctcaacccagggatcctggaagcagtgaccatgcagcacatcttcatgaacaacttccagctgtgcagtgagatcaacgagagagtggtccagcatttcgttcactgcatagagacccacggtcgaaatgtccagtatatcaagtttctccagactattgtcaaggccgaagggaaattcattaaaaagtgccaagacatggtcatggctgagcttgtcaattctggagaggacgtcctcgtgttctacaatgacagagcctctttccagactctgatccagatgatgcggtccgagcgtgaccggatggatgagaacagccctctcatgtaccacatccatctggtggagctcttggctgtgtgcacagagggcaagaatgtgtacacagagatcaagtgcaactccctgctcccgctcgatgacatcgttcgtgtggtcactcatgaagactgtatccccgaggttaagattgcttacatcaacttcctgaatcactgctatgtggacactgaggtggagatgaaggagatttacacaagtaatcacatgtggaagttgtttgagaatttcctcgtggacatctgcagggcctgtaacaacacaagtgacaggaagcatgcagactcggttctggagaagtacgtcactgaaatcgtgatgagcattgtcaccaccttcttcagctctcccttctcagaccagagtaccactctgcagactcgccagcctgtctttgtgcagctcctgcaaggcgtgttccgagtttaccactgcaactggttgatgccgagccagaaagcctcggtggagagctgtatccgcgtgctctctgacgtagccaagagccgggccatagccattcctgttgacctagacagccaagtcaacaacctcttcctgaagtcccacaacattgtgcagaaaacagccatgaactggcggttatcagcccgcaacgctgctcgtagagactctgtcctggcagcttccagagactaccgaaatattattgagaggttacaggacatcgtgtctgccctggaggaccgactcaggcccctggtccaggccgagctgtctgtgctcgtggatgttctacacagaccggaactgcttttccctgagaacacggatgccaggaggaaatgcgaaagtggaggtttcatctgcaagctcataaaacacaccaagcagctgctagaggagaatgaagagaaactgtgcattaaagtcttacagaccctcagggaaatgatgaccaaagatagaggctatggagaaaagcaaatttccattgatgaattggaaaatgccgagctgccacagccaccggaagctgagaactccacagagcaggagcttgaaccaagtccacccctgaggcaactggaagaccataaaaggggtgaggcactccggcaaattttggtcaaccgttactatggaaacatcagaccttcaggaagaagagagagccttaccagctttggcaatggtccactatcaccaggaggacccagcaagcctgggggaggagggggaggtcctgggtccggctccacaagccggggtgagatgagcctggctgaggttcaatgtcatctcgacaaggaaggcgcctccaatctggtcatcgacctcataatgaatgcatccagcgaccgagtattccacgaaagcattctcctggccatcgcacttctggaaggaggcaacaccactatccagcactcatttttctgccggctgacggaagataagaaatcagagaagttcttcaaggtcttttacgatcgaatgaaggtggcccagcaggaaatcaaggcgacagtgaccgtgaacaccagcgatttgggaaacaaaaagaaagatgatgaagtggaccgggatgccccatcccggaagaaagccaaagagcccacaacacagataacagaagaggtccgggatcagctcttggaagcatctgctgccaccagaaaagcctttaccaccttccggagggaggccgaccctgatgaccattaccaatctggggagggcacccaggctacaacggacaaagccaaggatgacctagagatgagtgcagtcatcaccatcatgcagcctatcctgcgcttcctgcagctcctgtgtgaaaaccacaaccgcgatctgcagaatttccttcgttgccaaaataataagaccaactacaacttggtgtgtgagacactgcagtttctggactgtatctgtgggagcacaactggaggccttggtcttctcggactgtacataaatgaaaagaatgtagctcttatcaaccaaaccctggagagtctgactgagtactgtcaagggccttgccatgaaaaccagaactgcatcgccacccacgagtccaatggcatcgatatcatcacagccctcatccttaacgatatcaaccctctggggaagaaacggatggacctggtgttagaactgaagaacaatgcttcaaagctactcctggccatcatggaaagcagacacgatagtgaaaatgcagagaggatcctgtacaacatgaggcccaaggagctggtggaagtgatcaagaaggcctacatgcaaggcgaagtggagtttgaggatggggagaacggtgaggatggagcagcctcccccaggaacgtgggccataacatctacatcctcgctcaccagttggctcggcataacaaagaacttcaaaccatgctgaaacctggaggccaggtggatggcgatgaagctctggagttctatgcgaagcacacagcacaaattgagattgtcagactggaccgaactatggaacagattgtcttccctgtgcccagcatctgtgaatttctgactaaggaatcgaaacttcgaatatattacaccacagagagggatgagcaaggcagcaagatcaatgacttcttcctgcgctccgaggacctctttaatgagatgaactggcagaagaaacttcgagcccagcctgtcttgtactggtgcgcccgaaacatgtctttctggagcagcatctccttcaacctggccgtcctgatgaacctgctggtggcgtttttctacccatttaaaggagtgagaggaggaacactagagccgcactggtccggcctcctgtggacagccatgctcatctctctggcaattgtcattgcgctgcccaagccccatggcatccgggccttaattgcttctacaatcctacgactgatattttcagttggattacagcccacactgtttctgctgggagctttcaatgtctgcaataaaatcatcttcctgatgagctttgtgggcaactgtgggaccttcaccagaggctaccgagccatggttctggacgttgagttcctctatcatttgctgtatctactgatctgtgccatgggcctcttcgtacatgagtttttctatagtttgctgctttttgatttagtgtacagagaggagactttgcttaatgtcattaaaagtgtcacccgcaatggacggtccatcatcttgacagcggtcctggctctgattctggtttacctgttctcaattgtgggctacctgttcttcaaggatgactttatcttggaagtagacaggttgcccaatgagacagctggtccagaaactggtgagagtttggccaacgatttcctgtactctgatgtgtgccgggtagagacgggggagaactgcacctctccggcacccaaagaagagctgctccctgtggaagaaacggaacaggataaggagcacacgtgtgagacactgctcatgtgcatcgtcaccgttctgagtcacgggctgcggagtgggggcggagtaggagatgtgctcaggaagccatccaaagaggagcctctgtttgctgcaagggtgatctatgacctcctcttcttcttcatggtcatcatcattgtcctgaacctcatttttggggtcatcatcgacacctttgctgacctgaggagtgagaagcagaagaaggaagagatcttaaaaaccacgtgcttcatttgtggcttggaaagggacaagtttgacaataagaccgtcacctttgaagagcacattaaggaagaacacaacatgtggcactatctgtgcttcatcgtgctggtgaaagtgaaggactccaccgagtacaccgggcccgagagctacgtggcagagatgatcagggaaagaaaccttgattggttccccaggatgagagccatgtccctggtcagcagtgactctgaaggggaacagaacgagctgaggaacctgcaggagaagctggagtccaccatgaagctggtcaccaatctttctggccagctgtcagaactaaaggaccagatgacagaacaaaggaagcagaaacaaagaattggccttctaggacatcctcctcacatgaatgtcaacccacagcagccagcctaagcaaatgagacggagggactgtactcaaccctttttaaattattgtcagcgtgggtctggctcttggttctgatctgccctctaaggttacatgtgcgcttagtacatttgtaaatactcggttttgtattgtatgtatatgattgcaattctcagaggtttggactttcgtattgtaattagctctgttggcatggtgacttgtcactcctgccaaaaatattaaaaatgccttttttggaaggactacagaaagtacctgatttgcacttgaaccagattatagatttaaaagtatatgacatgtattttgtatttaaaactagaatagccagtatttatgttttttataaaactgtgcaatacaaattatgcaatcaccataactctgtaactcctgagtgtcttaagggagtgcgcatctttgaagctgatctgttaatacttatgtaataaatggttaaatatcaaatgatgatgctgctgccaaaattatattaatagcaagtttctggcccctgggcaattttgtaccttgtaattatcctatggtgatgctgtttctcgttgctaatggcattagtgcctcgtatcctagtgataactccaggtctgtgaaccattcaaacaacattcatttttgagacaaagcaactttagtttcaagggtaattttaagcttcaaaattaatcatttaaagtatttctttaagagagccatgttagaggctcacacgttagcttgaaaggagtcgatgaaataatttttttaaagggaactttttacacgacgtttggaataacagcatattgctgaccagtcagtgtcatctcccaggtgaattttgatgtcacgttatagtcaaatgagctagctgatggtttctagattctcttcatctgaaccaacatgatgcagtaggtgagaagttattatgcatatatacacatatacatccatataaggcaaagtaggagctgtccccttaggatgcatagctgcccccttagggtgcagagctcaacactgacagtgctgttctgagagagccacgtttgggttttatttctttgtcacatgatttcttttctggatgtgtgcaagtataacaggaagtgttttctacctgttgcttgttttgtacctgggtctcgttttactagaccatctctgcacaaaagtttaaaaaatgaaccgtaagcagagttctgaagcaagtcaagtttgtaagtgcatacctgaaattatttaataaatgacgcagaatact

SEQ ID NO: 23 - protein, 2710 aa, Artificial Sequence
  Description of Artificial Sequence; Note = synthetic construct
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLFMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSVLEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTAMNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYAEKGEALRQILVNRYYGNIRPSGRRESLTSFGNGPLSPGGPSKPGGGGGGPGSGSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRPIILTAALALILVYLFSIVGYLFFKDDFILEVDRLPNETAGPETGESLANDFLYSDVCRVETGENCTSPAPKEELLPVEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 24 - protein, 6 aa, Artificial Sequence
  Description of Artificial Sequence; note = synthetic construct
GXGXXG

SEQ ID NO: 25 - protein, 4 aa, Artificial Sequence
  Description of Artificial Sequence; note = synthetic construct
RRDS

SEQ ID NO: 26 - protein, 4 aa, Artificial Sequence
  Description of Artificial Sequence; note = synthetic construct
RRES

SEQ ID NO: 27 - protein, 4 aa, Artificial Sequence
  Description of Artificial Sequence; note = synthetic construct
RRDE

SEQ ID NO: 28 - protein, 4 aa, Artificial Sequence
  Description of Artificial Sequence; note = synthetic construct
RREE

SEQ ID NO: 29 - protein, 4 aa, Artificial Sequence
  Description of Artificial Sequence; note = synthetic construct
RREA

SEQ ID NO: 30 - protein, 4 aa, Artificial Sequence
  Description of Artificial Sequence; note = synthetic construct
RRDA

SEQ ID NO: 31 - protein, 48 aa, Artificial Sequence
  Description of Artificial Sequence; note = synthetic construct
GYGEKQISIDELENAELPQPPEAENSTEELEPSPPLRQLEDHKRGEAL

SEQ ID NO: 32 - protein, 9 aa, Artificial Sequence
  Description of Artificial Sequence; note = synthetic construct
GYGEKGEAL